(19) United States
(12) Patent Application Publication
(10) Pub. No.: US 2006/0121564 A1
(43) Pub. Date: Jun. 8, 2006

(54) Novel expression vector suitable for expression of recombinant protein at low temperature

(75) Inventors: Nobutaka Nakashima, Hokkaido (JP); Tomohiro Tamura, Hokkaido (JP)

Correspondence Address: FOLEY AND LARDNER LLP;SUITE 500, 3000 K STREET NW, WASHINGTON, DC, 20007, US

(73) Assignee: Nationalnstitute of Advanced Industrial Science and Technology, Tokyo (JP)

(21) Appl. No.: 10/524,193

(22) PCT Filed: Aug. 11, 2003
(86) PCT No.: PCT/JP03/10209

§ 371 Date: Feb. 11, 2005

(30) Foreign Application Priority Data
Aug. 12, 2002 (JP) 2002-235008

(51) Int. Cl. C12P 21/06; C12N 1/20; C12N 15/74; C07K 14/195

(52) U.S. Cl. 435/69.1; 435/471; 435/252.3; 530/350

(57) ABSTRACT

An inducible expression vector which can express a protein at low temperature and a method for expressing a protein at low temperature using this vector are provided. The inducible expression vector can express a gene encoding a protein whose expression product, inhibits proliferation of the host cell under medium to high temperature conditions exceeding about 15° C. in a host cell which can proliferate under low temperature conditions.

### TECHNICAL FIELD

The present invention relates to an expression vector which can inducibly express a foreign gene in a bacterium of genus Rhodococcus.

The present invention also relates to an inducible expression vector which can express a recombinant protein in a host cell at low temperature and a method for expressing a recombinant protein at low temperature using this vector. Furthermore, the present invention relates to an inducible expression vector for a bacterium of genus Rhodococcus which vector can inducibly express a gene encoding a protein whose expression product inhibits proliferation of the host cell under medium to high temperature conditions exceeding about 15° C. in a bacterium of genus Rhodococcus which can proliferate under low temperature conditions and a method for expressing a recombinant protein whose expression product inhibits proliferation of the host cell under medium to high temperature conditions exceeding about 15° C. at low temperature using a bacterium of genus Rhodococcus which contains this vector and can proliferate under low temperature conditions.

### BACKGROUND ART

Currently, in order to prepare in mass a protein derived from a eukaryote as a recombinant, expression systems using E. coli as a host are widely used (Weickert et al., Curr. Opin. Biotechnol. 7 494-499 (1996); Baneyx, Curr. Opin. Biotechnol. 10 411-421 (1999)). E. coli is a mesophile, and although it grows at 18 to 37° C., culturing temperature for expressing the recombinant protein should also be within the above-mentioned temperature range. However, since the protein derived from a eukaryote shows activity in the same temperature range, some proteins, when made to express in E. coli as a recombinant, may inhibit the growth of E. coli, and consequently, significant quantity of recombinant protein may not be obtained.

Besides those using E. coli, expression systems using eukaryotic cells such as Saccharomyces cerevisiae and Pichia pastoris (Cereghino and Cregg, Curr. Opin. Biotechnol. 10 422-427 (1999)), Sf9 cell (Miller, Curr. Opin. Genet. Dev. 3 97-101 (1993)) as a host are known but these systems cannot express the protein efficiently either unless the culturing temperature is around 30° C., and the production thereof may be difficult for the similar reason. For example, when producing a foreign protein using insect cell Sf9 usually used for the production of recombinant protein, the optimal temperature for the production is about 28° C., and the lowest temperature is about 18° C. (Agathos et al., Ann. N.Y. Acad. Sci. 589 372-398 (1990), Faber et al., Yeast 11 1331-1344 (1995)). In the meantime, when producing a foreign protein using yeast (Pichia pastoris), the optimal temperature for the production is about 30° C., and the lowest temperature is about 15° C. (Brock et al., J. Membr. Biol. 180 147-161 (2001); Sarramegna et al., Protein Expr. Purif. 24 212-220 (2002)). That is, suitable growth temperature range of the insect cell Sf9 is about 18° C. or higher, and the suitable growth temperature range of the yeast is about 15° C. or higher. In addition, when these cells are used as a host, some proteins may be modified with oligosaccharide etc., which causes inconvenience in the subsequent functional analyses such as 3D structure analysis.

### DISCLOSURE OF THE INVENTION

An object of the present invention is to express a protein which cannot be expressed in the other recombinant protein expression systems including E. coli expression systems. For example, the present invention is directed to express a recombinant protein at low temperature which protein cannot be expressed under medium to high temperature conditions exceeding 15° C. in a recombinant host cell such as E. coli.

Another object of the present invention is to inductively express a foreign recombinant protein using a bacterium of genus Rhodococcus.

In order to solve the above-mentioned problem, it is considered to be effective to make it expressed at low temperature to suppress the activity of the recombinant protein. In E. coli, an expression system using a low-temperature inducible promoter at 15 to 16° C. is an example which produced a recombinant protein at the lowest temperature (JP Patent Publication (Kohyo) No.10-503090A (1998), Mujacic et al., Gene 238 325-332 (1999)). In addition, production of a recombinant protein at 15 to 18° C. is production of a recombinant protein at the lowest temperature known ever for insect cell and yeast as mentioned above. Therefore, it was considered that to make the recombinant protein to be expressed at or lower than 15° C. to 18° C., the lowest temperatures at which recombinant proteins can be expressed using conventionally known host cells express, preferably around 4° C., was effective. However, since the growth is difficult and protein production is impossible at or lower than 15° C., particularly around 4° C. for any of the above-mentioned host cells, use of an expression system employing a bacterium which can grow around 4° C. as a host is considered to be suitable. Accordingly, the present inventors have tried to solve the problems by establishing an inducible expression vector using a bacterium of genus Rhodococcus as a host and capable of expressing a foreign protein in a wide temperature range (from 4° C. to around 32° C.).

Rhodococcus erythropolis (Larkin et al., Antonie van Leeuwenhoek 74 133-153 (1998)) is an actinomycete growing in a wide temperature range from 4° C. to 35° C. and easy to be studied in biogenetics since a composite vector capable of autonomous replication in both cell species of this bacterium and E. coli (De Mot et al., Microbiology 143 3137-3147 (1997)) has been developed.

In addition, as for the bacteria of genus Rhodococcus in general, composite vectors for E. coli have been developed (JP Patent Publication (Kokai) No.5-64589A (1993), JP Patent Publication (Kokai) No.8-56669A (1996)), and a general-purpose expression vector which constitutively expresses a foreign gene also exists (JP Patent Publication (Kokai) No. 10-248578A (1998)).

However, in order to make the protein expressed rapidly at low temperature with efficiency, development of an inducible expression vector which easily enables strict and powerful regulation of expression of the protein is essential. That is, cells are allowed to proliferate at 30° C. with the expression suppressed first and then the temperature is lowered to 4° C. to induce expression. To date, however, there has been no report of such an inducible expression vector for this bacillus, and it was considered effective to divert an inducible expression system derived from those for other kinds of bacteria.

Streptomyces coelicolor is a kind of actinomycete as well as Rhodococcus erythropolis, and a series of gene clusters whose expression is inducible by the addition of an antibiotic thiostrepton are known for this bacterium (Murakami et al., J. Bacteriol. 171 1459-1466 (1989)). It has been known that TipA gene, one of these genes, encodes a protein consisted of 253 amino acids, and this TipA protein covalently binds to thiostrepton, and acts on its own promoter domain as a TipA-thiostrepton complex to powerfully promote the transfer from its own structural gene (Holmes et al., EMBO J. 12 3183-3191 (1993), Chiu et al., Biochemistry 35 2332-2341 (1996)). In addition, an inducible expression vector using the TipA structural gene and TipA gene promoter was also developed, and there is an example which made a foreign protein expressed within genus Streptomyces (Enguita et al., FEMS Microbiol. Lett. 137 135-140 (1996)). It is expected that if a vector having a gene cluster comprised of TipA structural gene and a structural gene of the target protein linked downstream to the TipA gene promoter introduced is constructed in Rhodococcus erythropolis as well, the vector can be an inducible expression vector in the same manner as in the case of a bacterium of genus Streptomyces. There has been, however, no such a report.

Moreover, if the production of a recombinant protein is enabled at low temperature about 15° C. or lower, particularly at 4° C., it will be considered that not only the protein which inhibits the proliferation of a host is enabled but also the following advantages will be obtained.

When E. coli is made to express a recombinant protein at 37° C., it may form an aggregated inactive protein called inclusion body. Many examples are known, however, that even the same protein is produced as an active and soluble protein when the expression temperature is lowered to below 30° C. (Schein and Noteborn, Bio/Technology 6 291-294 (1988); Piatak et al., J. Biol. Chem. 263 4837-4843 (1988); Schirano and Shibata, FEBS Lett. 271 128-130 (1990); Vasnia and Baneyx, Protein Expr. Purif. 9 211-218 (1997); Lin et al., Protein Expr. Purif. 1 169-176 (1990)). Therefore, if an expression system is constructed at low temperature of about 15° C. or lower, particularly around 4° C., this problem requiring solubilization is considered to be also solved.

Furthermore, a low temperature of about 15° C. or lower, particularly around 4° C. is supposed to be also favorable for the production of proteins derived from psychrotrophic bacteria whose optimal growth temperature range is 20° C. or lower, heterothermic animals living under a low-temperature environment and plants living under a low-temperature environment. This is because it is considered that these proteins are not expressed as active proteins when the temperature is too high. Although there is only one example in which α-amylase derived from a psychrotrophic bacterium is expressed using a psychrotrophic bacterium as a host (Tutino at al., Extremophiles 5 257-264 (2001)), the case was not with an expression inducible vector and it is considered to be difficult to quickly produce the protein in mass.

Thus the present inventors have intensively studied on the construction of an expression vector which can inducibly express a foreign protein in a bacterium of genus Rhodococcus and an expression vector which can inducibly express a foreign protein at low temperature about 15° C. or lower, and have completed the present invention.

That is, the present invention is as follows.
(1) An expression vector which can inducibly express a foreign gene with an inducing substance in a host cell and can be expressed at a temperature below the suitable growth temperature range of a host other than said host.
(2) An expression vector which can inducibly express a foreign gene with an inducing substance in a host cell and can be expressed at a temperature of 15° C. or lower.
(3) The expression vector according to (1) or (2) which can be expressed at 4° C.
(4) The expression vector according to any one of (1) to (3) wherein the host cell is a bacterium of genus Rhodococcus.
(5) The expression vector according to (4) wherein the bacterium of genus Rhodococcus is selected from the group consisting of R. erythropolis, R. fascians and R. opacus.
(6) The expression vector according to any one of (1) to (5) wherein the inducing substance is thiostrepton.
(7) The expression vector according to any one of (1) to (6) wherein the foreign gene encodes a protein which inhibits proliferation of the host cell under medium to high temperature conditions exceeding 15° C.
(8) The expression vector according to any one of (1) to (7) wherein the vector comprises a promoter sequence whose expression can be regulated by an inducing substance and a multicloning site to which a foreign gene can be introduced.
(9) A transformant comprising an expression vector according to any one of (1) to (8).
(10) A method for producing protein using an expression vector according to any one of (1) to (8).
(11) An inducible expression vector which can inducibly express a gene encoding a protein which inhibits proliferation of the host cell when it is expressed at a temperature within the range of suitable growth temperature of the host cell in another host cell having a suitable growth temperature range lower than the suitable growth temperature of said host cell using an inducing substance.
(12) The expression vector according to (11) which can be expressed at 4° C.
(13) The expression vector according to (11) or (12) wherein the host cell is a bacterium of genus Rhodococcus.
(14) The expression vector according to (13) wherein the bacterium of genus Rhodococcus is selected from the group consisting of R. erythropolis, R. fascians and R. opacus.
(15) The expression vector according to any one of (11) to (14) wherein the inducing substance is thiostrepton.
(16) The expression vector according to any one of (11) to (15) wherein the vector comprises a promoter sequence whose expression can be regulated by an inducing substance and a multicloning site to which a foreign gene can be introduced.
(17) A transformant comprising an expression vector according to any one of (11) to (16).
(18) A method for producing a protein using an expression vector according to any one of (1 1) to (16).
(19) An expression vector which can inducibly express a foreign gene using an inducing substance in a bacterium of genus Rhodococcus.
(20) The expression vector according to (19) wherein the bacterium of genus Rhodococcus is selected from the group consisting of R. erythropolis, R. fascians and R. opacus.
(21) The expression vector according to (19) or (20) wherein the inducing substance is thiostrepton.
(22) An expression vector according to any one of (19) to (21) wherein the vector comprises an expression cassette comprising a TipA gene promoter sequence, the first multicloning site to which a foreign gene can be introduced and a transcription termination sequence, an inducer cassette comprising the second promoter sequence and TipA gene, a DNA region essential to autonomous replication of a plasmid for a bacterium of genus Rhodococcus and a thiostrepton resistance gene.
(23) A transformant of a bacterium of genus Rhodococcus comprising an expression vector according to any one of (19) to (22).
(24) A method for producing a protein using an expression vector according to any one of (19) to (22).
(25) An inducible expression vector for a bacterium of genus Rhodococcus which can inducibly express a gene encoding a protein whose expression product inhibits proliferation of the host cell under medium to high temperature conditions exceeding 15° C. in a bacterium of genus Rhodococcus which can proliferate under low-temperature conditions.
(26) An inducible expression vector for a bacterium of genus Rhodococcus whose expression can be induced in a bacterium of genus Rhodococcus in which a foreign gene can be multiplied under low-temperature conditions, wherein the vector comprises an expression cassette comprising a TipA gene promoter sequence, the first multicloning site to which a foreign gene can be introduced and a transcription termination sequence, an inducer cassette comprising the second promoter sequence and TipA gene, a DNA region essential to autonomous replication of a plasmid for a bacterium of genus Rhodococcus and a thiostrepton resistance gene.
(27) The inducible expression vector for a bacterium of genus Rhodococcus according to (26) wherein the vector further comprises a DNA region essential to autonomous replication of a plasmid for E. coli and can be reproduced in E. coli.
(28) The inducible expression vector for a bacterium of genus Rhodococcus according to (26) or (27) wherein the TipA gene promoter is TipA-LG10 promoter.
(29) The inducible expression vector for a bacterium of genus Rhodococcus according to any one of (26) to (28) wherein the vector is selected from the group consisting of pTip-NH1 having a nucleotide sequence represented by SEQ ID No. 106, pTip-NH2 having a nucleotide sequence represented by SEQ ID No. 107, pTip-CH1 having a nucleotide sequence represented by SEQ ID No. 108, pTip-CH2 having a nucleotide sequence represented by SEQ ID No. 109, pTip-LNH1 having a nucleotide sequence represented by SEQ ID No. 110, pTip-LNH2 having a nucleotide sequence represented by SEQ ID No. 111, pTip-LCH1 having a nucleotide sequence represented by SEQ ID No. 112, pTip-LCH2 having a nucleotide sequence represented by SEQ ID No. 113, pTip-CH1.1, pTip-CH2.1, pTip-LCH1.1 and pTip-LCH2.1.
(30) The inducible expression vector for a bacterium of genus Rhodococcus according to any one of (25) to (29) wherein the bacterium of genus Rhodococcus is selected from the group consisting of R. erythropolis, R. fascians and R. opacus.
(31) A transformant of a bacterium of genus Rhodococcus comprising an inducible expression vector for a bacterium of genus Rhodococcus according to any one of (25) to (30).
(32) A process for producing a protein whose expression product inhibits proliferation of the host cell under medium to high temperature conditions exceeding 15° C. wherein the process comprises introducing an inducible expression vector for a bacterium of genus Rhodococcus according to any one of (25) to (30) comprising as a foreign gene a gene encoding a protein difficult to be expressed under medium to high temperature conditions exceeding 15° C. into a bacterium of genus Rhodococcus which can proliferate at low temperature; and culturing the above-mentioned bacteria into which an inducible expression vector for a bacterium of genus Rhodococcus is introduced using a culture medium comprising thiostrepton under low-temperature.
(33) The process for producing a protein at low temperature according to (32) wherein the protein difficult to be expressed under medium to high temperature conditions exceeding 15° C. is a protein which inhibits proliferation of the host cell under medium to high temperature conditions exceeding 15° C.
(34) The process for producing a protein at low temperature according to (32) wherein the protein difficult to be expressed under medium to high temperature conditions exceeding 15° C. is a protein which forms an inactive inclusion body in E. coli when it is made expressed under medium to high temperature conditions exceeding 15° C.
(35) A process for producing a protein derived from psychrotrophic bacteria, heterothermic animals or plants living under a low-temperature environment wherein the process comprises introducing an inducible expression vector for a bacterium of genus Rhodococcus according to any one of (25) to (30) comprising a gene encoding a protein derived from psychrotrophic bacteria, heterothermic animals or plants living under a low-temperature environment into a bacterium of genus Rhodococcus which can proliferate at low temperature; and culturing the above-mentioned bacteria into which an inducible expression vector for a bacterium of genus Rhodococcus is introduced using a culture medium comprising thiostrepton under low-temperature.
(36) A process for screening a protein difficult to be expressed under medium to high temperature conditions exceeding 15° C. wherein the process comprises introducing an inducible expression vector for a bacterium of genus Rhodococcus according to any one of (25) to (30) comprising a foreign gene into a bacterium of genus Rhodococcus which can proliferate at low temperature; culturing the above-mentioned bacteria into which an inducible expression vector for a bacterium of genus Rhodococcus is introduced using a culture medium comprising thiostrepton under medium to high temperature conditions exceeding 15° C. and under low-temperature condition; and selecting a gene which can be expressed only under low-temperature condition equal to or below 15° C.
(37) The process for screening a protein difficult to be expressed under medium to high temperature conditions exceeding 15° C. according to (36) wherein the protein difficult to be expressed under medium to high temperature conditions exceeding 15° C. is a protein which inhibits proliferation of the host cell under medium to high temperature conditions exceeding 15° C.
(38) A process for screening a protein difficult to be expressed under medium to high temperature conditions exceeding 15° C. wherein the process comprises selecting a gene which does not express or inhibits proliferation of E. coli when it is introduced and made expressed under medium to high temperature conditions exceeding 15° C.; and then introducing an inducible expression vector for a bacterium of genus Rhodococcus according to any one of (25) to (30) comprising said gene as a foreign gene into a bacterium of genus Rhodococcus which can proliferate at low temperature; and selecting a gene which can be expressed when cultured under low-temperature condition using a culture medium comprising thiostrepton.
(39) The process for screening a protein according to (38) wherein the protein difficult to be expressed under medium to high temperature conditions exceeding 15° C. is a protein which inhibits proliferation of E. coli at 30° C. or higher.
(40) The process for screening a protein according to (38) wherein the protein difficult to be expressed under medium to high temperature conditions exceeding 15° C. is a protein which forms an inclusion body when it is made expressed under medium to high temperature conditions exceeding 15° C.
(41) The process for screening a protein according to (38) wherein the protein difficult to be expressed under medium to high temperature conditions exceeding 15° C. is a protein which inhibits proliferation of the host cell under medium to high temperature conditions exceeding 15° C.
(42) A protein difficult to be expressed under medium to high temperature conditions exceeding 15° C. obtained by a screening process according to any one of (36) to (41).

Hereafter, the present invention is described in detail.

### 1. CONSTRUCTION OF EXPRESSION VECTOR OF THE PRESENT INVENTION

The expression vector of the present invention is capable of autonomous replication in a cell which can proliferate at low temperature, and which can inducibly express a foreign gene incorporated into the vector, i.e., an inducible expression vector.

The cell which can proliferate at low temperature is not limited, as long as it is a cell which can proliferate at low temperature, and any microbes such as E. coli and yeast, insect cells, mammals cells, etc. can be used. In view of secure proliferation at low temperature, bacteria belonging to genus Rhodococcus, preferably R. erythropolis, R. fascians, R. opacus, etc. are included. Among these three kinds of a bacterium of genus Rhodococcus, R. erythropolis has a highest proliferation rate at 4° C., and the other two are inferior thereto. However, in the production of a protein using a vector of the present invention, cells can be proliferated at a temperature suitable for proliferation and then transferred to low temperature condition so that the protein can be inductively expressed and produced. Accordingly, as long as the cells can express and produce a foreign protein at 4° C., proliferation rate does not matter and all of the three kinds species belonging to genus Rhodococcus, i.e., R. erythropolis, R. fascians, and genus Rhodococcus of R. opacus can be suitably used.

Low temperature refers to a temperature lower than the optimal proliferation temperature of usual bacteria, and means a temperature from 4° C. to 18° C., preferably from 4° C. of 15° C., particularly preferably around 4° C. Although the suitable growth temperature range of usual bacteria can vary depending on the kind, it is about 15° C. to about 40° C., or about 18° C. to about 40° C., and temperature exceeding about 15° C. is referred to as medium to high temperature in this specification.

A foreign gene is a gene encoding the target protein to be expressed and produced using a vector of the present invention, and a gene encoding a protein derived from an organism other than the host cell. The foreign gene to be incorporated into a vector of the present invention is a gene encoding a protein which is difficult or impossible to be expressed at medium to high temperatures exceeding about 15° C. The protein which is difficult or impossible to be expressed at high temperature exceeding about 15° C. means a protein which expresses with low efficiency or does not express at all when it is attempted to be expressed at medium to high temperatures exceeding about 15° C. Examples of the proteins include genes encoding proteins such as a protein which cannot be expressed in the range of optimal growth temperature of the host cell but can be expressed at a temperature lower than the suitable growth temperature of the microbe when the identical or different kind of a microbe is used as the host cell; a protein which is lethal to the host cell when it is expressed at a temperature within the suitable growth temperature range of the host microbe but not lethal to the identical or different kind of microbe at a temperature lower than the suitable growth temperature of the microbe; a protein which inhibits proliferation of the host cell when it is expressed at a temperature within the suitable growth temperature range of the host cell but does not inhibit proliferation of the host cell at a temperature lower than the suitable growth temperature of the identical or different kind of host cell; a protein which forms an inactive protein aggregation called inclusion body when it is expressed at a temperature within the suitable growth temperature range of the host cell but becomes an active and soluble protein when expressed in the host cell at a temperature lower than the suitable growth temperature of the identical or different kind of host cell; a protein derived from psychrotrophic bacteria whose suitable growth temperature range is 20° C. or lower, heterothermic animals living under low-temperature environment and plants living under low-temperature environment etc.

When a particular gene is made expressed at medium to high temperatures exceeding about 15° C. in an expression system based on E. coli, or the gene is incorporated in an expression vector of the present invention and made expressed in a bacterium of genus Rhodococcus at medium to high temperatures exceeding about 15° C., and if the gene does not express or expresses in an amount significantly lower than the amount of the case that a foreign gene is incorporated in an expression vector of the present invention and made expressed in a bacterium of genus Rhodococcus at low temperature, the protein can be recognized as a protein which is difficult or impossible to be expressed at medium to high temperatures exceeding about 15° C.

For example, when a gene is made expressed using E. coli commonly used for ordinary expression production of a recombinant protein and it cannot be expressed at 18 to 37° C. which is the suitable growth temperature range of E. coli, or the protein is lethal to the E. coli, or inhibits proliferation of E. coli, or forms aggregation of inactive inclusion body, the gene of the above-mentioned protein can be introduced into Rhodococcus erythropolis and Rhodococcus erythropolis can be proliferated at a low temperature of 4 to 18° C. to enable an efficient production of the above-mentioned protein in large quantities. In addition, when a gene is made expressed using Rhodococcus erythropolis at a temperature exceeding about 15° C. and it cannot be expressed, or the protein is lethal to the Rhodococcus erythropolis, or inhibits proliferation of Rhodococcus erythropolis, it is also possible to perform an efficient production of the above-mentioned protein in large quantities by proliferating Rhodococcus erythropolis at a low temperature of 4 to 18° C.

Although the type of the protein which inhibits proliferation of the host cell at medium to high temperatures exceeding about 15° C. is not limited, examples thereof include proteins illustrated in the below-mentioned Examples. The genes encoding such a protein can be incorporate into a site having a multicloning site incorporated downstream the below-mentioned promoter.

The vector which can inducibly express a foreign gene means a vector in which expression of a foreign gene incorporated by a certain process is induced. For example, it is possible to construct an inducible expression vector by incorporating a promoter whose expression can be induced with a specific regulating substance into a vector. Such a promoter includes a promoter which can be specifically induced by introducing a chemical agent, i.e., an inducing substance into the culture medium of the host cell, for example, TipA gene promoter which is a thiostrepton inducing promoter. The target protein can be expressed in large quantities by adding a chemical agent which induces expression of the protein after sufficiently proliferating the host cell in which has been introduced a vector incorporating such an inductive promoter at the temperature suitable for proliferation of the cell at a temperature of 15 to 18° C. or higher. In addition, TipA gene encoding TipA protein and any suitable promoters such as ThcA gene promoter which induces the expression of TipA gene may be incorporated. When the host cells are bacteria belonging to genus Rhodococcus, since these bacteria are susceptible to thiostrepton, thiostrepton resistance gene or the like which confers resistance to thiostrepton is incorporated.

Moreover, the expression vector of the present invention may contain a drug resistance gene.

Furthermore, the vector may be a composite vector (shuttle vector) so that it may be compatible to two or more host cells. Examples of such a vector include a vector which can be introduced into any of bacteria belonging to E. coli and genus Rhodococcus, and can express a foreign gene in these host cells. When building such a vector, it is necessary to incorporate DNA regions essential to autonomous replication of a plasmid in each host cell. For example, as for a composite vector suitable for bacteria belonging to E. coli and genus Rhodococcus, ColE1 sequence may be incorporated as a DNA region essential to autonomous replication of a plasmid for E. coli and RepA and RepB genes may be incorporated as DNA regions essential to autonomous replication of a plasmid for bacteria belonging to genus Rhodococcus. Such a composite vector can be reproduced in large quantities using E. coli.

The expression vector of the present invention contains at least a DNA sequence having the first promoter activity and the first multicloning site for incorporating a foreign gene. Furthermore, it may contain DNA region(s) essential to autonomous replication of the first plasmid, the first drug resistance gene, a foreign gene linked to the first multicloning site, and the first transcription termination sequence. TipA gene promoter can be mentioned as a DNA sequence having the first promoter activity and when TipA gene promoter is contained, TipA gene and the second promoter sequence such as ThcA gene promoter for expressing TipA gene, and the second transcription termination sequence downstream from the TipA gene are contained. The TipA gene promoter can be a sequence modified therefrom such as TipA-LG10 promoter. Furthermore, when TipA gene promoter inducible expression system is contained and the host cell is a bacterium of genus Rhodococcus, thiostrepton resistance gene should be contained in order to confer thiostrepton resistance to the bacterium of genus Rhodococcus.

The DNA sequence having a promoter activity, the foreign gene and the transcription termination sequence constitute an expression cassette, and TipA gene and the promoter for TipA gene expression constitute an inducer cassette.

The expression vector for a bacterium of genus Rhodococcus of the present invention may express the protein not only at low temperature but also at medium to high temperatures exceeding 15° C. as long as the protein itself can be expressed at medium to high temperatures exceeding 15° C.

pTip vector shown in FIG. 9 can be mentioned as an expression vector of the present invention and the vector includes pTip-NH1, pTip-NH2, pTip-CH1, pTip-CH2, pTip-LNH1, pTip-LNH2, pTip-LCH1 and pTip-LCH2 depending on the structure of the multicloning site as shown in FIG. 9a. The sequences of pTip-NH1, pTip-NH2, pTip-CH1, pTip-CH2, pTip-LNH1, pTip-LNH2, pTip-LCH1 and pTip-LCH2 vectors are indicated as the SEQ ID Nos. 106 to 113, respectively.

In addition, expression vectors of the present invention include pTip-CH1.1, pTip-CH2.1, pTip-LCH1.1 and pTip-LCH2.1 in which BglII and XhoI sites are separated in pTip-CH1, pTip-CH2, pTip-LCH1, and pTip-LCH2 so that the reading frame of the multicloning site after the XhoI site is in frame with the reading frame of a commercial pET vector (Novagen).

The vector of the present invention can be easily constructed by following the description of the below-mentioned Examples and vector construction diagrams of FIGS. 1 to 8.

### 2. USE OF VECTOR OF THE PRESENT INVENTION

Use of vector of the present invention enables to produce a protein which is difficult or impossible to be expressed at medium to high temperatures exceeding about 15° C. Examples of the proteins include a protein which cannot be expressed at a temperature within the suitable growth temperature range of the host cell but can be expressed at a temperature lower than the suitable growth temperature of the cell when the identical or different kind of host cell is used as the host cell; a protein which is lethal to the host cell when it is expressed at a temperature within the suitable growth temperature range of the host cell but not lethal to the identical or different kind of host cell at a temperature lower than the suitable growth temperature of the host cell; a protein which inhibits proliferation of the host cell when it is expressed at a temperature within the suitable growth temperature range of the host cell but does not inhibit proliferation of the host cell at a temperature lower than the optimal growth temperature of the identical or different kind of host cell; a protein which forms an inactive protein aggregation called inclusion body when it is expressed at a temperature within the suitable growth temperature range of the host cell but becomes an active and soluble protein when expressed in the host cell at a temperature lower than the optimal growth temperature of the identical or different kind of host cell; a protein derived from psychrotrophic bacteria whose suitable growth temperature range is 20° C. or lower, heterothermic animals living under low-temperature environment and plants living under low-temperature environment.

The above-mentioned proteins can be expressed by incorporating a gene encoding such a protein into a multicloning site of the expression vector of the present invention using a suitable restriction enzyme, transforming the host cell by this vector, and culturing the host cell under low-temperature conditions. The host cell should be a cell which can proliferate at low temperature, and a bacterium belong to genus Rhodococcus, preferably R. erythropolis, R. fascians, R. opacus, etc. can be mentioned. Although these cells can proliferate at low temperature, suitable temperature for proliferation is 15° C. or higher, more preferably 18° C. or higher and particularly preferably around 30° C., and the protein can be expressed by sufficiently proliferating the cells at a temperature suitable for proliferation before expressing the protein incorporated in the gene and subsequently placing them under low-temperature condition to express the protein utilizing the inducible promoter function contained in the vector along with a suitable chemical agent.

When the vector of the present invention contains TipA gene promoter, expression of protein is induced by adding thiostrepton to the culture medium. Under the present circumstances, thiostrepton may be added so that the final concentration may be at or more than 0.1 μg/ml, preferably at or more than 1 μg/ml. However, growth will be deteriorated when the final concentration exceeds 10 μg/ml.

Use of vector of the present invention enables to screen a protein which is difficult or impossible to be expressed at medium to high temperatures exceeding about 15° C. Examples of the proteins include a protein which cannot be expressed at a temperature within the suitable growth temperature range of the host cell but can be expressed at a temperature lower than the suitable growth temperature of the cell when the identical or different kind of host cell is used as the host cell; a protein which is lethal to the host cell when it is expressed at a temperature within the suitable growth temperature range of the host cell but not lethal to the identical or different kind of host cell at a temperature lower than the suitable growth temperature of the host cell; a protein which inhibits proliferation of the host cell when it is expressed at a temperature within the suitable growth temperature range of the host cell but does not inhibit proliferation of the host cell at a temperature lower than the optimal growth temperature of the identical or different kind of host cell; and a protein which forms an inactive protein aggregation called inclusion body when it is expressed at a temperature within the suitable growth temperature range of the host cell but becomes an active and soluble protein when expressed in the host cell at a temperature lower than the optimal growth temperature of the identical or different kind of host cell.

For example, poly(A)⁺RNA is extracted from a suitable tissue of a suitable animal species, cDNA is synthesized, and incorporated in an expression vector. Subsequently, host cells such as E. coli are transformed using this vector, an expression library is constructed, proliferated and expressed at 30° C., and the gene is isolated from the clone in which the gene is incorporated and whose proliferation has been inhibited whereby genes for encoding a protein which is lethal to the host cell when it is expressed at a temperature within the suitable growth temperature range of the host cell but not lethal to the identical or different kind of host cell at a temperature lower than the suitable growth temperature of the host cell and a protein which inhibits proliferation of the host cell when it is expressed at a temperature within the suitable growth temperature range of the host cell but does not inhibit proliferation of the host cell at a temperature lower than the optimal growth temperature of the identical or different kind of host cell are selected. For this purpose, a promoter inducible by a suitable chemical agent is incorporated in the expression vector and clones may be selected by which proliferation of the host cells is inhibited when the expression is induced by the chemical agent while they can proliferate when the expression is not induced. Subsequently, isolated genes are incorporated in the expression vector of the present invention and Rhodococcus erythropolis is transformed by this recombinant expression vector, proliferated and expressed at a low temperature from 4 to 15° C., and clones are selected which express the gene without being inhibited from proliferation whereby the gene encoding the above-mentioned protein can be screened. Alternatively, genes of the cDNA library are incorporated in the expression vector of the present invention and Rhodococcus erythropolis is transformed by this recombinant expression vector, proliferated and expressed at a low temperature or medium to high temperatures exceeding about 15° C., and clones are selected which express the incorporated gene without being inhibited from proliferation or clones are selected in which the gene being expressed upon expression induction is incorporated whereby the gene encoding the above-mentioned protein can be screened.

The present invention encompasses the proteins obtained by the above-mentioned screening which are difficult or impossible to be expressed at medium to high temperatures exceeding about 15° C. These proteins can be exemplified by the proteins illustrated in the following Example.

This specification incorporates the contents disclosed in the specification and/or drawings of Japanese Patent Application No. 2002-235008, from which the priority of the present application is claimed.

### BRIEF DESCRIPTION OF THE DRAWINGS

FIG. 1 is a diagram illustrating the construction of plasmid pHN136 which constitutes the backbone of the inducible expression vector. The positions of the restriction enzyme recognition site and the structural gene are shown in the drawing. The numbers indicate the number of base pairs (kilo base pair: kb);

FIG. 2 is a diagram illustrating the construction of plasmid pHN143 having a thiostrepton resistance gene. The positions of the restriction enzyme recognition site and the structural gene are shown in the drawing. The numbers indicate the number of base pairs (kilo base pair: kb). CIAP means Calf Intestine Alkaline Phosphatase and Blu. means a blunt end;

FIG. 3 is a diagram illustrating the construction of plasmid pHN62 having an inducer cassette. The positions of the restriction enzyme recognition site and the structural gene are shown in the drawing. The numbers indicate the number of base pairs (kilo base pair: kb). Blu. means a blunt end;

FIG. 4 is a diagram illustrating the construction of plasmid pHN153 having an expression cassette. The positions of the restriction enzyme recognition site and the structural gene are shown in the drawing. The numbers indicate the number of base pairs (kilo base pair: kb). CIAP means Calf Intestine Alkaline Phosphatase and Blu. means a blunt end;

FIG. 5 is a diagram illustrating the construction of plasmid pHN169 having a tetracycline resistance gene. The positions of the restriction enzyme recognition site and the structural gene are shown in the drawing. The numbers indicate the number of base pairs (kilo base pair: kb). CIAP means Calf Intestine Alkaline Phosphatase and Blu. means a blunt end;

FIG. 6 is a diagram illustrating the construction of inducible expression vector plasmids pHN170 and pHN171 which have PIP as a reporter gene. The positions of the restriction enzyme recognition site and the structural gene are shown in the drawing. The numbers indicate the number of base pairs (kilo base pair: kb). CIAP means Calf Intestine Alkaline Phosphatase. A series of steps is shown by dividing into two parts in FIG. 6, but there is an overlap in the two portions so that the order of the steps may become clear;

FIG. 7 is a diagram illustrating the construction of inducible expression vector plasmids pTip-NH1, pTip-CH1, pTip-LNH1 and pTip-LCH1 having a multicloning site. The positions of the restriction enzyme recognition site and the structural gene are shown in the drawing. The numbers indicate the number of base pairs (kilo base pair: kb). A series of steps is shown by dividing into two parts in FIG. 7, but there is an overlap in the two portions so that the order of the steps may become clear;

FIG. 8 is a diagram illustrating the construction of inducible expression vector plasmids pTip-NH2, pTip-CH2, pTip-LNH2 and pTip-LCH2 having a multicloning site. The positions of the restriction enzyme recognition site and the structural gene are shown in the drawing. The numbers indicate the number of base pairs (kilo base pair: kb). A series of steps is shown by dividing into two parts in FIG. 8, but there is an overlap in the two portions so that the order of the steps may become clear;

FIG. 9a is a) a diagram showing the map of pTip-NH1, pTip-CH1, pTip-LNH1, pTip-LNH1, pTip-NH2, pTip-CH2, pTip-LNH2 and pTip-LCH2. The function of each domain and the map of the plasmids are shown;

FIG. 9b shows b) the DNA sequence from TipA gene promoter sequence of pTip-NH1, pTip-LNH1, or from TipA-LG10 promoter sequence to the multicloning site and the transcription termination sequence of ThcA gene;

FIG. 9c shows c) the DNA sequence from TipA gene promoter sequence of pTip-CH1, pTip-LCH1, or from TipA-LG10 promoter sequence to the multicloning site and the transcription termination sequence of ThcA gene;

FIG. 9d shows d) the DNA sequence from TipA gene promoter sequence of pTip-NH2, pTip-LNH2, or from TipA-LG10 promoter sequence to the multicloning site and the transcription termination sequence of ThcA gene;

FIG. 9e shows e) the DNA sequence from TipA gene promoter sequence of pTip-CH2, pTip-LCH2, or from TipA-LG10 promoter sequence to the multicloning site and the transcription termination sequence of ThcA gene;

FIG. 10 is a diagram showing the map of pTip-CH1.1, pTip-LCH1.1, pTip-CH2.1 and pTip-LCH2. 1;

FIG. 11 is a diagram illustrating the construction of the control plasmids pHN172 and pHN173 for PIP activity measurement. The positions of the restriction enzyme recognition site and the structural gene are shown in the drawing. The numbers indicate the number of base pairs (kilo base pair: kb). CIAP means Calf Intestine Alkaline Phosphatase. pHN170 has both of an “expression cassette” and an “inducer cassette” whereas pHN173 has only an “expression cassette” and pHN172 does not have neither of the cassettes;

FIG. 12 is a figure showing the result of the measurement 1 of the PIP activity using the inducible expression vector;

FIG. 13 is a graph showing the result of the measurement 2a of the PIP activity using the inducible expression vector;

FIG. 14 is a graph showing the result of the measurement 2b of the PIP activity using the inducible expression vector;

FIG. 15 is a figure showing the result of the measurement 3 of the PIP activity using the inducible expression vector;

FIG. 16 is a photograph in which the result of purification 1 of the foreign protein using the inducible expression vector;

FIG. 17 is a photograph in which the result of purification 2 of the foreign protein using the inducible expression vector;

FIG. 18 is a photograph in which the result of purification 3a of the foreign protein using the inducible expression vector;

FIG. 19 is a photograph in which the result of purification 3b of the foreign protein using the inducible expression vector;

FIG. 20 is a figure showing the list of proteins which inhibit proliferation of E. coli at 30° C.;

FIG. 21 is a figure showing expression of the foreign protein using Rhodococcus erythropolis and E. coli as a host;

FIG. 22 is a figure showing TipA gene promoter sequence; and

FIG. 23 is a figure showing an improvement of the RBS sequence (WTRBS) in TipA gene promoter to LG10RBS.

### BEST MODE FOR CARRYING OUT THE INVENTION

The present invention will be described in more detail by way of the following examples. The technical scope of the present invention, however, is not limited to these examples.

### EXAMPLE 1

Isolation of the plasmid derived from Rhodococcus erythropolis which can autonomously replicate in a bacterium of genus Rhodococcus and determination of a partial DNA sequence thereof

In order to create a composite vector for Rhodococcus erythropolis and E. coli, small endogenous plasmids occurring in a bacterium of genus Rhodococcus were first searched. As a result, there was one observed in Rhodococcus erythropolis JCM2895 strain. This plasmid was named pRE2895. Isolation of the plasmid and determination of a partial DNA sequence thereof are specifically described below.

pRE2895 was purified using QIAprep Spin MiniprepKit (product of QIAGEN) from cell bodies obtained by culturing Rhodococcus erythropolis JCM2895 strain using 5 ml LB culture medium (1% Difco Bacto Tryptone, 0.5% Difco Yeast Extract, 1.0% sodium chloride) at 30° C. for 30 hours. This procedure was conducted following the manufacturer's instructions except that 5 μl of lysozyme (100 mg/ml) was added followed by incubation at 37° C. for 30 minutes after the sample was dispersed in 250 μl of buffer P1 and before 250 μl of buffer P2 was added.

The above-mentioned DNA sample was processed with restriction enzyme EcoRI and subjected to 1.0% agarose gel electrophoresis (100 V, 30 minutes), and existence of one DNA fragment of about 5.4 kb was observed.

This DNA fragment of about 5.4 kb was excised from the gel, and purified with QIAquick Gel Extraction Kit (product of QIAGEN) following the manufacturer's instructions. The obtained EcoRI fragment was subcloned into the EcoRI site of plasmid pBluescript II SK(+) (product of STRATAGENE) according to an ordinary method (Sambrook et al., Molecular Cloning: a laboratory manual, 2nd edition (1989), Cold Spring Harbor Laboratory Press, Cold Spring Harbor, N.Y.), and this plasmid was named pHN79.

The nucleotide sequence of pHN79 was sequenced by using DNA sequencer ABI PRISM (R) 3100 Genetic Analyzer (product of ABI Co. Ltd.) with both of Reverse and M13-20 primers (products of STRATAGENE) following the manufacturer's instructions by about every 400 bases, respectively. Homology search revealed that the DNA region derived from Rhodococcus erythropolis JCM 2895 strain which was subcloned into pHN79 agreed with pN30, a circular DNA of 5403 base pairs which was registered in GenBank under Accession Number AF 312210 by 99.8% of the sequence.

Although the full length sequence of the isolated pRE2895 was not determine, it was highly homologous with pN30, and since the restriction enzyme cleavage map thereof agreed with the map expected from pN30, it was expected that they are homologous throughout the whole plasmid. In the meantime, pN30 was highly homologous with endogenous plasmid pAL5000 isolated from Mycobacterium fortuitum 002 strain (Rauzer et al., Gene 71 315-321 (1988); Stolt and Stoker, Microbiology 142 2795-2802 (1996)) and pFAJ2600 isolated from Rhodococcus erythropolis NI 86/21 strain (De Mot et al., Microbiology 143 3137-3147 (1997)) and it was supposed that they are autonomously replicated by a similar mechanism. Since a region containing presumed RepA gene, presumed RepB gene and presumed replication origin was sufficient for the autonomous replication of pAL5000 in each bacterium, it was considered sufficient for the autonomous replication of pRE2895 in a bacterium of genus Rhodococcus to merely incorporate a similar region into an expression vector.

### EXAMPLE 2

Construction of Vector Plasmid pHN136

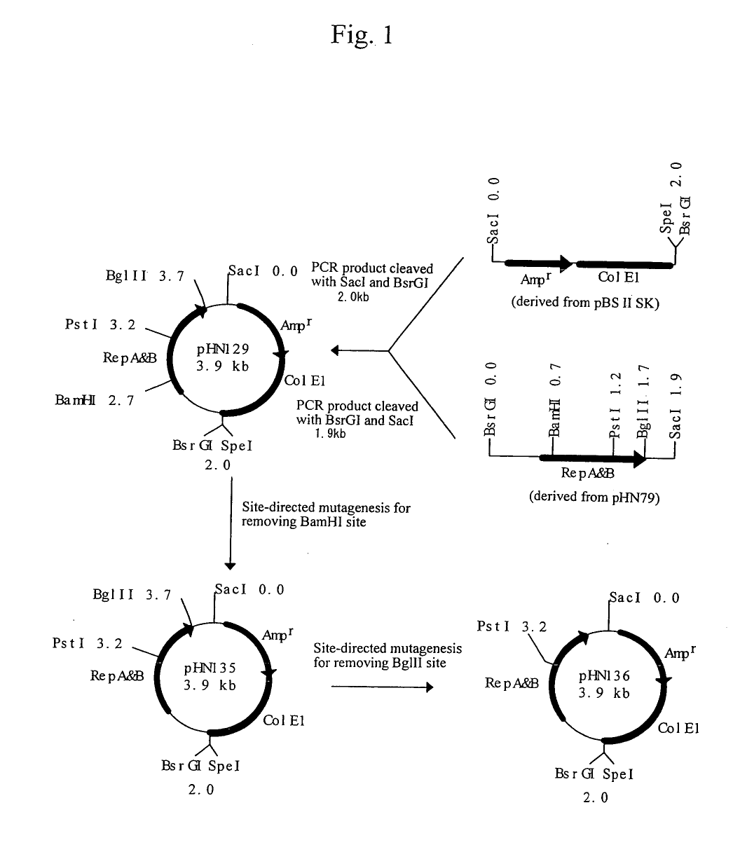

The following procedure was conducted in order to create a composite vector for both the bacteria using a part of pRE2895 isolated in Example 1 and a part of plasmid which can autonomously replicate in E. coli (FIG. 1).

Amplification of DNA was conducted using plasmid pBluescript II SK (−) (product of STRATAGENE) as a template along with synthetic oligodeoxyribonucleotide primers (hereinafter abbreviated as primers) of SEQ ID Nos. 1 and 2 in the Sequence Listing according to polymerase chain reaction method (hereinafter abbreviated as PCR: Saiki et al., Science, 239 487-491 (1988)). The enzyme used for PCR is Pfu turbo (product of STRATAGENE). Consequently, amplified DNA of 2.0 kb containing an ampicillin resistance gene (designated as Amp^{r} in the drawings) and a ColE1 sequence region required for autonomous replication in E. coli was obtained. Double digestion of this DNA fragment was carried out with restriction enzymes SacI and BsrGI, and the products were subjected to 1.0% agarose gel electrophoresis (100 V, 30 minutes). The DNA fragment was excised, and purified with QIAquick Gel Extraction Kit according to the manufacturer's instructions.

A primer which amplifies the region considered to be required for autonomous replication in a bacterium of genus Rhodococcus was designed based on the sequence of pN30 (Example 1). The sequences of these primers are shown by SEQ ID Nos. 3 and 4 in the Sequence Listing. PCR was performed using plasmid pHN79 as a template along with both the primers, and amplified DNA having 1.9 kb was obtained. Double digestion of this DNA fragment was carried out with restriction enzymes BsrGI and SacI, and the products were subjected to 1.0% agarose gel electrophoresis (100 V, 30 minutes). The DNA fragment was excised, and purified in the same manner as in the above-mentioned method.

The two above-mentioned purified DNA fragments were ligated using DNA Ligation Kit Ver.2 (product of TAKARA SHUZO CO., LTD.) following the manufacturer's instructions, and the obtained plasmid was named pHN 129.

Next, the following procedure was performed in order to remove the restriction enzyme recognition sites BamHI and SalI which exist in pHN129. First, amplification by PCR was performed using pHN129 as a template along with primers of SEQ ID Nos. 5 and 6 in the Sequence Listing. A DNA fragment of 0.5 kb obtained by carrying out double digestion of this PCR fragment by BglII and PstI was subcloned into BamHI and PstI sites of pHN129. As a result, BamHI recognition site was removed without substituting any encoded amino acid although the connecting site of BglII and BamHI was within the open reading frame (hereinafter abbreviated as ORF) of a presumed RepA gene. In addition, SalI recognition site located very close to the BamHI recognition site was also removed simultaneously with the BamHI recognition site in the primer of SEQ ID No. 5 since the primer was designed so that SalI recognition site was removed and any encoded amino acid would not be substituted. This plasmid was named pHN135.

Next, the following procedure was performed in order to remove the restriction enzyme recognition site BglII which exists in pHN135. First, amplification by PCR was performed using pHN135 as a template along with primers of SEQ ID Nos. 5 and 6 in the Sequence Listing: A DNA fragment of 0.5 kb obtained by carrying out double digestion of this PCR fragment by PstI and BamHI was subcloned into PstI and BglII sites of pHN135. As a result, BglII recognition site was removed without substituting any encoded amino acid although the connecting site of BamHI and BglII was within the ORF of a presumed RepB gene. This plasmid was named pHN136.

### EXAMPLE 3

Construction of Vector Plasmid pHN143

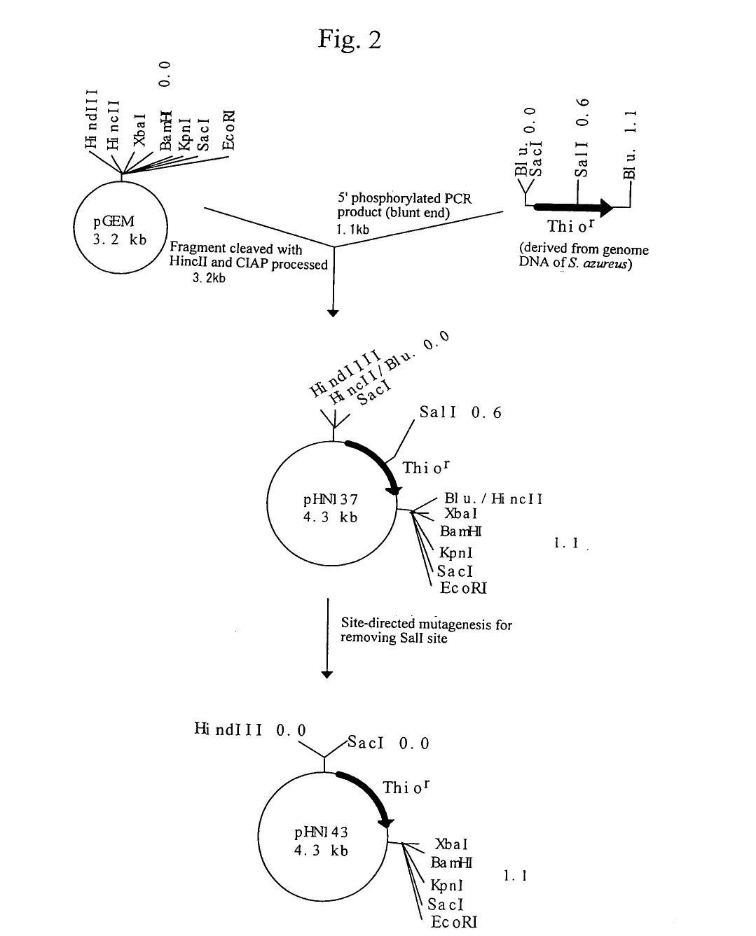

Since Rhodococcus erythropolis is susceptible to thiostrepton, an antibiotic used for induction expression of protein, it should be imparted with resistance to this substance. Thus, the present inventors decided to incorporate tsr gene, a thiostrepton resistance gene which Streptomyces azureus has (Bibb et al., Mol. Gen. Genet. 199 26-36 (1985)—designated as Thio^{r} in the drawing) into the composite vector. It was already reported that this gene functions within Rhodococcus erythropolis and confers thiostrepton resistance (Shao and Behki, Lett. Appl. Microbiol. 21 261-266 (1995)). Below, isolation of this gene is described specifically (FIG. 2).

First, the genome DNA of Streptomyces azureus JCM4217 strain used as the template of PCR was prepared as follows. This strain cultured at 30° C. by using 5 ml of SB culture medium (1% Difco Bacto Tryptone, 0.5% Difco Yeast Extract, 0.5% sodium chloride, 0.1% Glucose, 5 mM magnesium chloride, 0.5% glycine) was suspended into 500 μI of SET buffer (75 mM sodium chloride, 25 mM EDTA (pH 8.0), 20 mM Tris-HCI (pH 7.5)). 5 μl of a lysozyme solution (100 mg/ml) was added thereto and incubated at 37° C. for 30 minutes. Then 14 μl of a protease K solution (20 mg/ml) and 60 μl of a sodium dodecylsulfate solution (10%) were added, and after mixing well, incubated at 55° C. for 2 hours. Then, 200 μl of a sodium chloride solution (5 M) and 500 μl of chloroform were added, and rotation agitation was carried out at room temperature for 20 minutes. Centrifugal separation was carried out and 700 μl of supernatant was removed. This was dried after isopropanol precipitation, and dissolved in 50 μl of a TE solution (10 mM Tris-HCl (pH 8.0) and 1 mM EDTA (pH 8.0)).

Amplification by PCR was performed using as a template the genome DNA of Streptomyces azureus JCM4217 strain purified as mentioned above along with primers of SEQ ID Nos. 7 and 8 in the Sequence Listing. Consequently, an amplified DNA of 1.1 kb containing a thiostrepton resistance gene was obtained. This DNA fragment was obtained by using platinum Pfx DNA polymerase (product of Gibco BRL), and therefore the end thereof is a blunt end. This DNA fragment was purified and after phosphorylated at 5′ end by T4-polynucleotide kinase according to an ordinary method (Sambrook et al., Molecular Cloning: a laboratory manual, 2nd edition (1989), Cold Spring Harbor Laboratory Press, Cold Spring Harbor, N.Y.) subcloned into HincII site of plasmid pGEM-3Zf(+)(product of Promega) (the direction of subcloning was a HindIII recognition site-tsr gene ORF-EcoRI recognition site from 5′ end of DNA). This plasmid was named pHN137.

Next, the following procedure was performed in order to remove the restriction enzyme recognition site SalI which exists in pHN137. First, amplification by PCR was performed using plasmid pHN137 as a template along with primers of SEQ ID Nos. 9 and 10 in the Sequence Listing. Platinum Pfx DNA polymerase was used for this PCR. A DNA fragment of 0.6 kb obtained by digesting one end of this PCR fragment by HindIII was purified and phosphorylated at 5′ end of the side of a blunt end by T4-polynucleotide kinase following an ordinary method. In the meantime, amplification by PCR was performed using plasmid pHN137 as a template along with primers of SEQ ID Nos. 11 and 12 in the Sequence Listing. Platinum Pfx DNA polymerase was used for this PCR. A DNA fragment of 0.5 kb obtained by digesting one end of this PCR fragment by EcoRI was purified and further phosphorylated at 5′ end of the blunt end side by T4-polynucleotide kinase following an ordinary method. As a result of subcloning these two PCR fragments into HindIII and EcoRI sites of plasmid pGEM-3Zf(+) simultaneously, SalI recognition site was removed without substituting any encoded amino acid although the connecting site of the blunt ends was within the ORF of tsr gene. This plasmid was named pHN143.

### EXAMPLE 4

Construction of Vector Plasmid pHN62

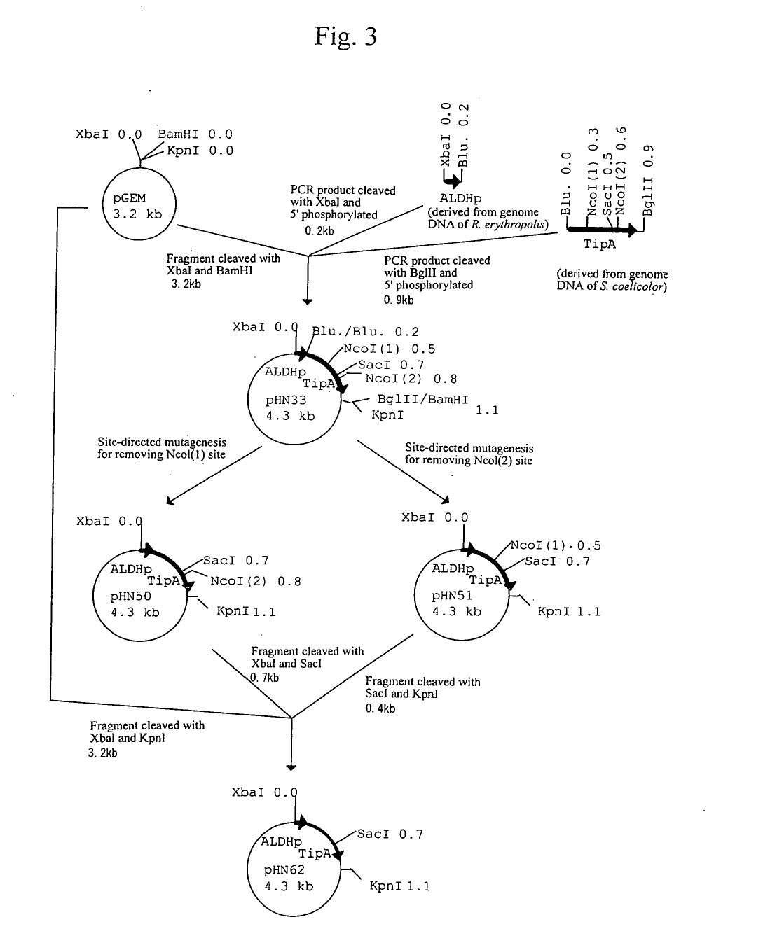

In order to carry out induced type expression by thiostrepton, TipA protein there must be present in a bacterium of genus Rhodococcus. Therefore, a constitutive promoter was separated from Rhodococcus erythropolis and the structural gene encoding the TipA protein was linked to the downstream thereof (FIG. 3). As a promoter which functions constitutively, a promoter sequence of ThcA gene encoding the aldehyde dehydrogenase-like protein of Rhodococcus erythropolis (Nagy et al., J. Bacteriol. 177 676-687 (1995)) was used.

The genome DNA of Streptomyces coelicolor A3 (2) strain used as a template was purified in the similar manner as in the preparation procedure of genome DNA from Streptomyces azureus. The genome DNA of Rhodococcus erythropolis JCM3201 strain used as a template was purified in the similar manner as in the preparation procedure of genome DNA from Streptomyces azureus except culturing was perform by using 5 ml LB culture medium.

Amplification by PCR was performed using as a template the genome DNA of Streptomyces coelicolor A3 (2) strain purified as mentioned above along with primers of SEQ ID Nos. 13 and 14 in the Sequence Listing. Platinum Pfx DNA polymerase was used for this PCR. Consequently, DNA (designated as TipA in the drawing) containing ORF of TipA gene and the transcription termination sequence downstream thereof was obtained.

A DNA fragment of 0.9 kb obtained by digesting one end of this PCR fragment by BglII was purified and phosphorylated at 5′ end by the side of a blunt end by T4-polynucleotide kinase following an ordinary method. In the meantime, amplification by PCR was performed using as a template the genome DNA of Rhodococcus erythropolis JCM3201 strain along with primers of SEQ ID Nos. 15 and 16 in the Sequence Listing. Consequently, DNA containing a promoter sequence (designated as ALDHp in the drawing) of ThcA gene (Nagy et al., J. Bacteriol. 177 676-687 (1995)) encoding an aldehyde dehydrogenase-like protein was obtained. Platinum Pfx DNA polymerase was used for this PCR. A DNA fragment of 0.2 kb obtained by digesting one end of this PCR fragment by XbaI was purified and further phosphorylated at 5′ end of the blunt end side by T4-polynucleotide kinase following an ordinary method. As a result of subcloning these two PCR fragments into XbaI and BamHI sites of plasmid pGEM-3Zf(+) simultaneously, a plasmid containing ORF of TipA gene and the transcription termination sequence closely downstream from the promoter sequence of ThcA gene was obtained. This plasmid was named pHN33.

Next, the following procedure was performed in order to remove two restriction enzyme NcoI recognition sites (hereinafter designated as NcoI (1) and NcoI (2)) which exist in pHN33.

First, amplification by PCR was performed using plasmid pHN33 as a template along with primers of SEQ ID Nos. 9 and 17 in the Sequence Listing. Platinum Pfx DNA polymerase was used for this PCR. A DNA fragment of 0.5 kb obtained by digesting one end of this PCR fragment by XbaI was purified and phosphorylated at 5′ end of the side of a blunt end by T4-polynucleotide kinase following an ordinary method. In the meantime, amplification by PCR was performed using plasmid pHN33 as a template along with primers of SEQ ID Nos. 18 and 12 in the Sequence Listing. Platinum Pfx DNA polymerase was used for this PCR. A DNA fragment of 0.6 kb obtained by digesting one end of this PCR fragment by KpnI was purified and phosphorylated at 5′ end of the side of a blunt end by T4-polynucleotide kinase following an ordinary method. As a result of subcloning these two PCR fragments into XbaI and KpnI sites of plasmid pGEM-3Zf(+) simultaneously, the NcoI(l) recognition site was removed without substituting any encoded amino acid although the connecting site of the blunt ends was within the ORF of TipA gene. This plasmid was named pHN50.

Next, the following procedure was performed in order to remove the restriction enzyme recognition site NcoI (2) which exists in pHN33. First, amplification by PCR was performed using plasmid pHN33 as a template along with primers of SEQ ID Nos. 9 and 19 in the Sequence Listing. Platinum Pfx DNA polymerase was used for this PCR. A DNA fragment of 0.8 kb obtained by digesting one end of this PCR fragment by XbaI was purified and phosphorylated at 5′ end of the side of a blunt end by T4-polynucleotide kinase following an ordinary method. In the meantime, amplification by PCR was performed using pHN33 as a template along with primers of SEQ ID Nos. 20 and 12 in the Sequence Listing. Platinum Pfx DNA polymerase was used for this PCR. A DNA fragment of 0.3 kb obtained by digesting one end of this PCR fragment by KpnI was purified and phosphorylated at 5′ end of the side of a blunt end by T4-polynucleotide kinase following an ordinary method. As a result of subcloning these two PCR fragments into XbaI and KpnI sites of plasmid pGEM-3Zf(+) simultaneously, the NcoI(2) recognition site was removed without substituting any encoded amino acid although the connecting site of the blunt ends was within the ORF of TipA gene. This plasmid was named pHN5 1.

The following procedure was performed at the end. A DNA fragment of 0.7 kb obtained by double digestion of pHN50 by XbaI and SacI and a DNA fragment of 0.4 kb obtained by double digestion of pHN51 by SacI and KpnI were subcloned into XbaI and KpnI sites of plasmid pGEM-3Zf(+) simultaneously. As a result, a plasmid having a TipA gene lacking in both the NcoI(1) and NcoI(2) recognition sites was acquired, and this was named pHN62.

### EXAMPLE 5

Construction of Vector Plasmid pHN 153

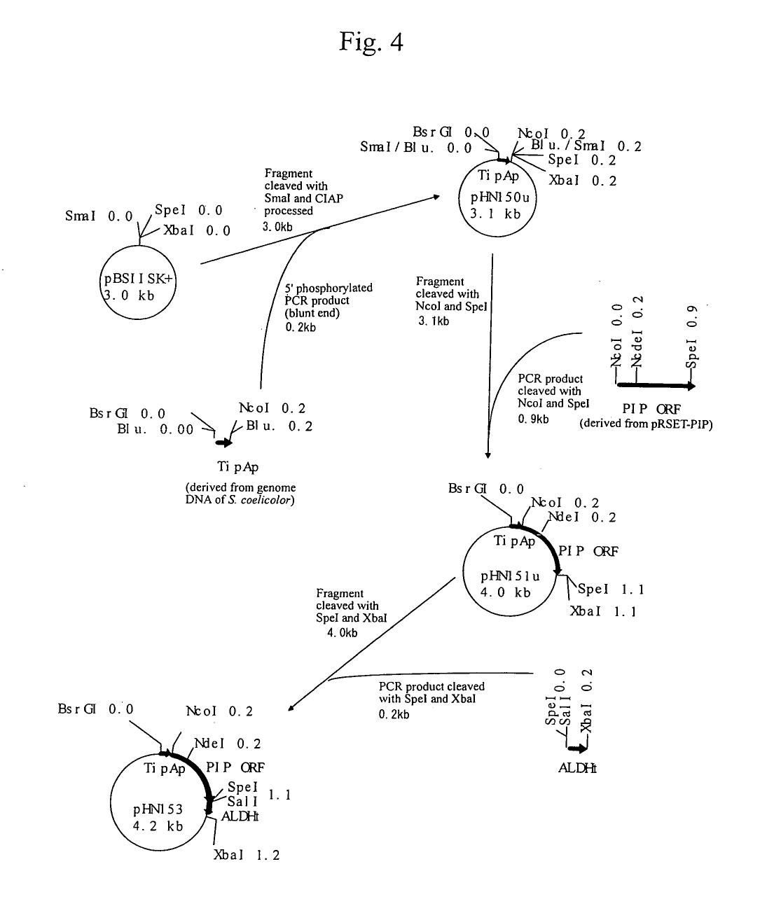

In order to confirm whether the target protein can be inducibly expressed, ORF of a gene encoding proline iminopeptidase derived from Thermoplasma acidophilum (Tamura et al., FEBS Lett. 398 101-105 (1996); hereinafter abbreviated as PIP) was linked downstream from the promoter of a TipA gene as a reporter gene ORF and a transcription termination sequence was linked further downstream therefrom to suppress the read-through of transcription. Details are specifically described below (FIG. 4).

Amplification by PCR was performed using as a template the genome DNA of the Streptomyces coelicolor A3 (2) strain purified in Example 4 along with primers of SEQ ID Nos. 21 and 22 in the Sequence Listing. Consequently, an amplified DNA of 0.2 kb containing a promoter sequence of TipA gene (designated as TipAp in the drawing) was obtained. Platinum Pfx DNA polymerase was used for this PCR. This fragment was purified and phosphorylated at 5′ end by T4-polynucleotide kinase following an ordinary method and subcloned into SmaI site of plasmid pBluescript II SK (+) (the direction of subcloning was a KpnI recognition site-TipA gene promoter sequence-SacI recognition site from 5′ end of DNA). This plasmid was named pHN150u.

Next, amplification by PCR was performed using plasmid pRSET-PIP (Tamura et al., FEBS Lett. 398 101-105 (1996)—hereinafter abbreviated as PIP) as a template along with primers of SEQ ID Nos. 23 and 24 in the Sequence Listing. The primer of SEQ ID No. 24 in the Sequence Listing is designed so that the termination codon of PIP gene is removed and a 6× His tag is attached to the C-terminal of the PIP protein for facilitating purification of the protein. The 6× His tag consists of six contiguous histidine residues and a protein fused with this exhibits high affinity to nickel ion, etc. Therefore, purification becomes easy by the metal chelate chromatography using nickel ion etc. (Crowe et al., Methods Mol. Biol. 31 371-387 (1994)). As a result of double digestion of a DNA fragment of 0.9 kb containing this PIP gene by restriction enzymes NcoI and SpeI and subcloning into NcoI and SpeI sites of pHN150u, a plasmid containing ORF of PIP gene immediately downstream from the promoter sequence of TipA gene was created and named pHN151u.

Next, amplification by PCR was performed using as a template the genome DNA of the Rhodococcus erythropolis JCM3201 strain purified in Example 4 along with primers of SEQ ID Nos. 25 and 26 in the Sequence Listing. Consequently, DNA containing the transcription termination sequence of ThcA gene (Nagy et al., J. Bacteriol. 177 676-687 (1995)—designated as ALDHt in the drawing) was obtained. Double digestion of this DNA fragment of 0.2 kb was carried out by restriction enzymes SpeI and XbaI, and the product was subcloned into SpeI and XbaI sites of pHN151u. Consequently, a plasmid containing ORF of PIP gene immediately downstream from the promoter sequence of TipA gene and further containing a transcription termination sequence of ThcA gene downstream therefrom was created, and named pHN153.

### EXAMPLE 6

Construction of Vector Plasmid pHN169

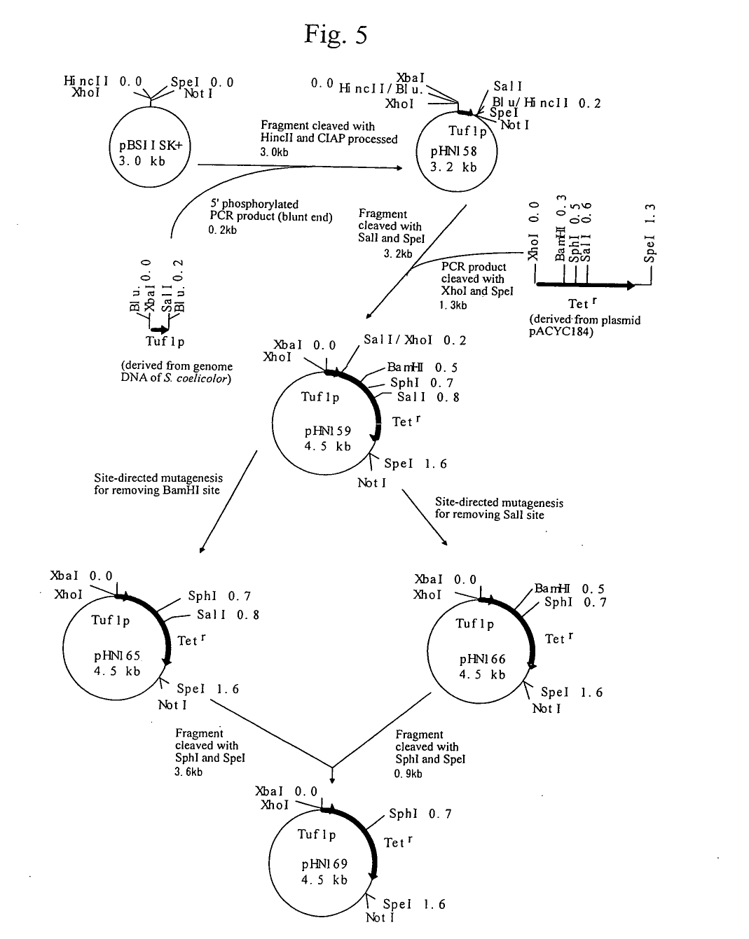

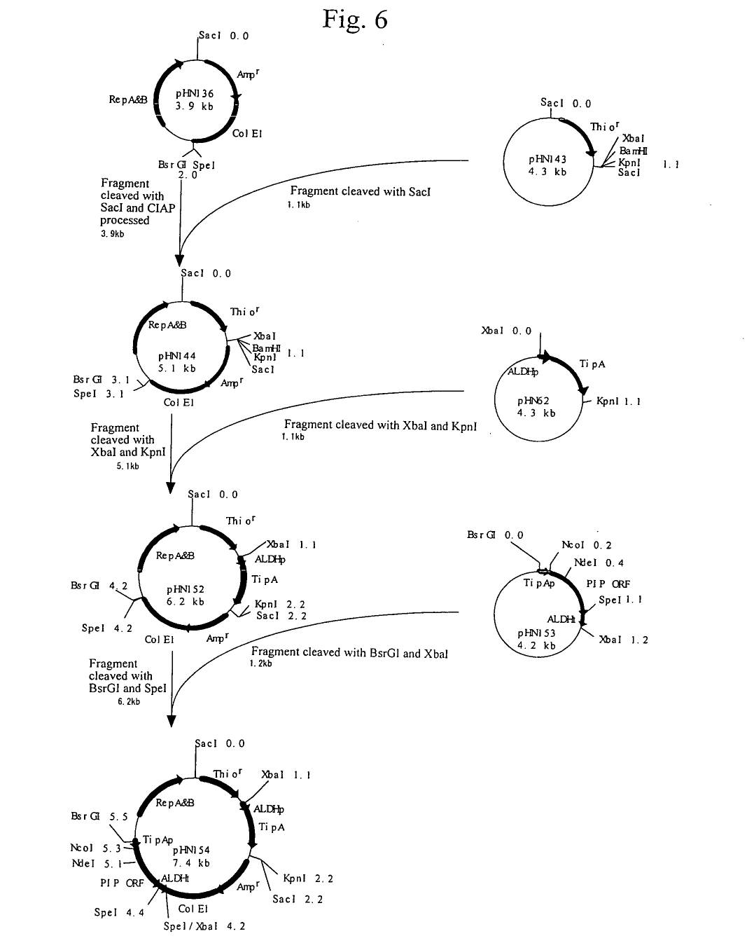

In order to carry out the transformation of Rhodococcus erythropolis by plasmid, a suitable transformation marker is needed. Therefore, the present inventors decided to connect and use a drug resistance gene downstream from a powerful promoter which functions within a bacterium of genus Rhodococcus. As a promoter, the present inventors selected to use Tuf1 gene promoter which encodes the elongation factor Tu derived from a bacterium of genus Streptomyces. This is because there has been a report that this promoter powerfully transcribes a downstream gene (Wezel et al., Biochim. Biophys. Acta 1219 543-547 (1994)). As a drug resistance gene, a tetracycline resistance gene which is easily available was used. Details are specifically described below (FIG. 5).

Amplification by PCR was performed using as a template the genome DNA of the Streptomyces coelicolor A3 (2) strain purified in Example 4 along with primers of SEQ ID Nos. 27 and 28 in the Sequence Listing. Consequently, an amplified DNA of 0.2 kb containing a promoter sequence of Tuf1 gene (designated as Tuflp in the drawing) was obtained. Platinum Pfx DNA polymerase was used for this PCR. This fragment was purified and phosphorylated at 5′ end by T4-polynucleotide kinase following an ordinary method and subcloned into HincII site of plasmid pBluescript II SK (+) (the direction of subcloning was a KpnI recognition site-Tuf1 gene promoter sequence-EcoRI recognition site from 5′ end of the DNA). This plasmid was named pHN158.

Next, amplification by PCR was performed using plasmid pACYC1 84 (Rose, Nucleic Acids Res. 16 355 (1988)) as a template along with primers of SEQ ID Nos. 29 and 30 in the Sequence Listing. Consequently, DNA containing a tetracycline resistance gene (designated as Tet^{r} in the drawing) was obtained. Double digestion of this DNA fragment of 1.3 kb was carried out by restriction enzymes XhoI and SpeI, and the product was subcloned into SalI and SpeI sites of pHN158. Consequently, a plasmid containing a tetracycline resistance gene was created immediately downstream of the promoter sequence of Tuf1 gene, and named pHN159.

Next, the following procedure was performed in order to remove the restriction enzyme recognition site BamHI which exists in pHN159. First, amplification by PCR was performed using plasmid pHN159 as a template along with primers of SEQ ID Nos. 31 and 32 in the Sequence Listing. Since the DNA fragment is Pfu turbo DNA polymerase, both ends were blunt ends. A DNA fragment of 0.5 kb obtained by digesting one end of this PCR fragment by XhoI was purified and phosphorylated at 5′ end of the side of the blunt end by T4-polynucleotide kinase following an ordinary method. In the meantime, amplification by PCR was performed using pHN159 as a template along with primers of SEQ ID Nos. 33 and 34 in the Sequence Listing. Pfu turbo DNA polymerase was used for this PCR. A DNA fragment of 1.1 kb obtained by digesting one end of this PCR fragment by NotI was purified and phosphorylated at 5′ end of the side of a blunt end by T4-polynucleotide kinase following an ordinary method. As a result of subcloning these two PCR fragments into XhoI and NotI sites of plasmid pBluescript II SK (+) simultaneously, the BamHI site was removed without substituting any encoded amino acid although the connecting site of the blunt ends was within the ORF of a tetracycline resistance gene. This plasmid was named pHN165.

Next, the following procedure was performed in order to remove the restriction enzyme recognition site SalI which exists in pHN159. First, amplification by PCR was performed using plasmid pHN159 as a template along with primers of SEQ ID Nos. 31 and 35 in the Sequence Listing. Pfu turbo DNA polymerase was used for this PCR. A DNA fragment of 0.8 kb obtained by digesting one end of this PCR fragment by XhoI was purified and phosphorylated at 5′ end of the side of a blunt end by T4-polynucleotide kinase following an ordinary method. In the meantime, amplification by PCR was performed using pHN159 as a template along with primers of SEQ ID Nos. 36 and 34 in the Sequence Listing. Pfu turbo DNA polymerase was used for this PCR. A DNA fragment of 0.8 kb obtained by digesting one end of this PCR fragment by NotI was purified and phosphorylated at 5′ end of the side of a blunt end by T4-polynucleotide kinase following an ordinary method. As a result of subcloning these two PCR fragments into XhoI and NotI sites of plasmid pBluescript II SK (+) simultaneously, the SalI recognition site was removed without substituting any encoded amino acid although the connecting site of the blunt ends was within the ORF of a tetracycline resistance gene. This plasmid was named pHN166.

The following procedure was performed at the end. A DNA fragment of 0.9 kb obtained by double digestion of pHN166 by SphI and SpeI was subcloned into SphI and SpeI sites of plasmid pHN165. As a result, a clone having a tetracycline resistance gene lacking in both the BamHI and SalI recognition sites was acquired, and this plasmid was named pHN169.

### EXAMPLE 7

Construction of Vector Plasmids pHN170 and pHN171

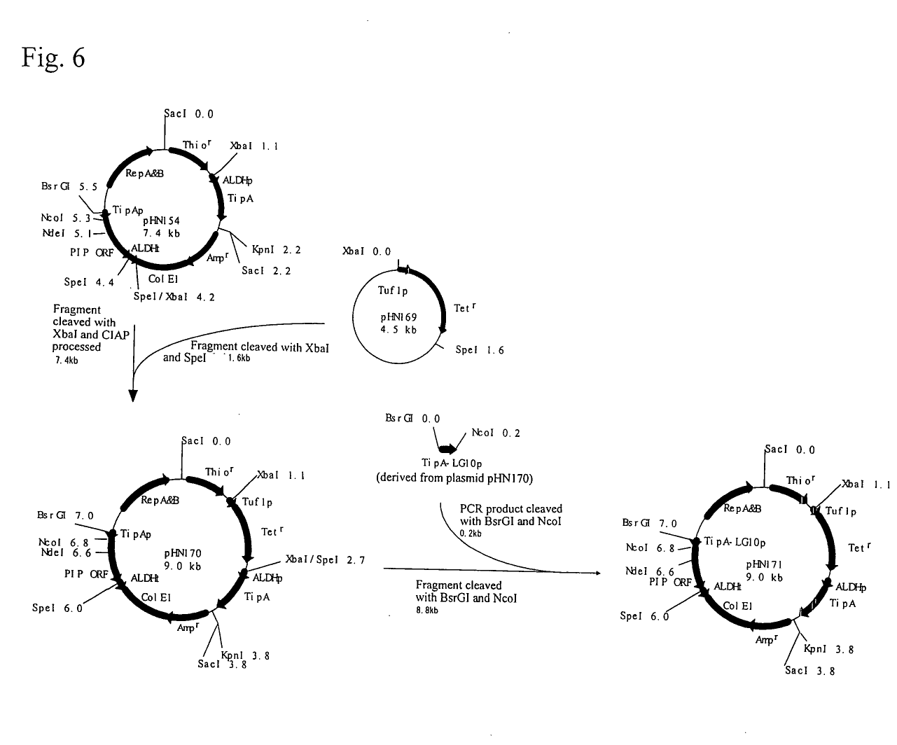

The following procedure was performed to link the gene clusters isolated in Examples 2 to 6 together and to construct an expression vector which can be induced in a bacterium of genus Rhodococcus (FIG. 6).

A DNA fragment of 1.1 kb obtained by digesting pHN143 by SacI was subcloned into SacI site of pHN136 (the direction of subcloning was a presumed RepB gene ORF-tsr gene ORF-ampicillin resistance gene ORF from 5′ end of the DNA). The resultant plasmid was named pHN144.

Next, a DNA fragment of 1.1 kb obtained by double digestion of pHN62 by XbaI and KpnI was subcloned into XbaI and KpnI sites of plasmid pHN144. The resultant plasmid was named pHN152.

Next, a DNA fragment of 1.2 kb obtained by double digestion of pHN153 by BsrGI and XbaI was subcloned into BsrGI and SpeI sites of plasmid pHN153. The resultant plasmid was named pHN154.

Next, a DNA fragment of 1.6 kb obtained by double digestion of pHN169 by XbaI and SpeI was subcloned into XbaI site of plasmid pHN154 (the direction of subcloning was tsr gene ORF-tetracycline resistance gene ORF-ThcA gene promoter sequence from 5′ end of the DNA). As a result, a plasmid containing PIP gene under control of the TipA gene promoter was created, and the resultant plasmid was named pHN170.

The ribosome binding site downstream of the TipA gene promoter was changed to a sequence which was derived from lambda phage gene10 and considered to be good in translation efficiency (Gold and Stormo, Methods Enzymol. 185 89-93 (1990)) for enhancing expression of recombinant protein (FIG. 6). Details are specifically described below.

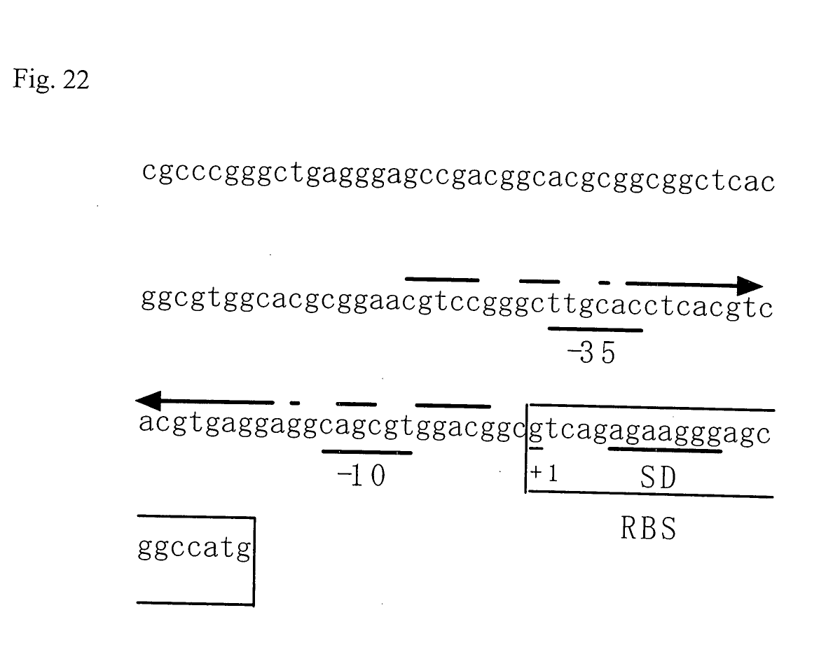

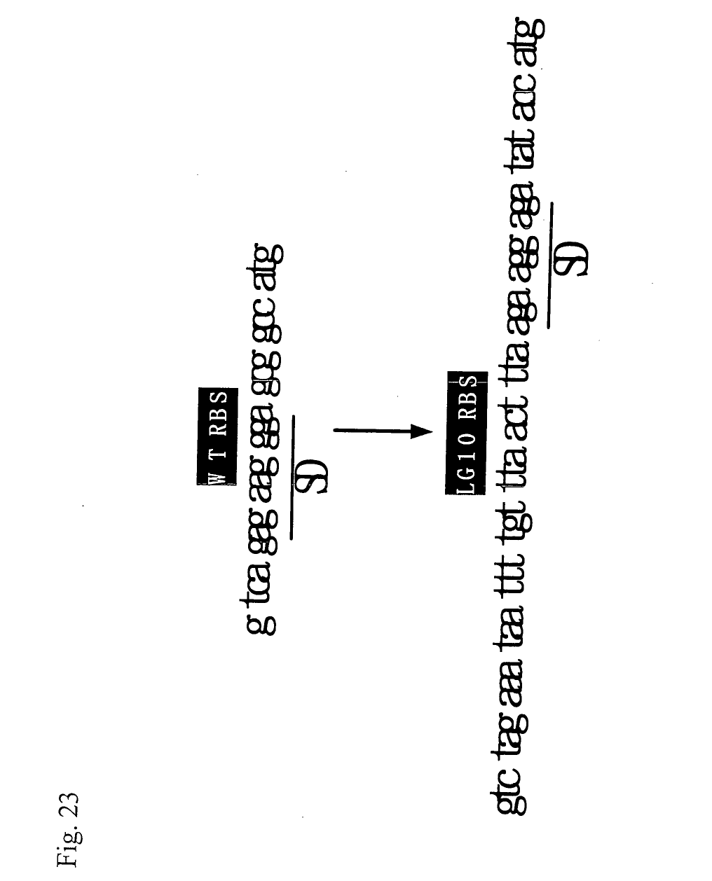

Amplification by PCR was performed using plasmid pHN170 as a template along with primers of SEQ ID Nos. 21 and 37 in the Sequence Listing. Consequently, a hybrid promoter consisting of the TipA gene promoter and the binding site derived from lambda phage gene10 (hereinafter designated as TipA-LG10 promoter: also designated TipA-LG10p in the drawing) was obtained. Double digestion of this DNA fragment of 0.2 kb was carried out with restriction enzymes BsrGI and NcoI, and the product was subcloned into BsrGI and NcoI sites of pHN170. As a result, a plasmid containing PIP gene under control of the TipA-LG10 promoter was created, and the resultant plasmid was named pHN171. The TipA promoter sequence is shown in FIG. 22 and an improvement of the ribosome binding site (RBS) sequence for the modification of the TipA promoter to the TipA-LG10 promoter is shown in FIG. 23.

### EXAMPLE 8

Construction of Vector Plasmid pTip-NH1, pTip-CH1, pTip-LNH1, and pTip-LCH1

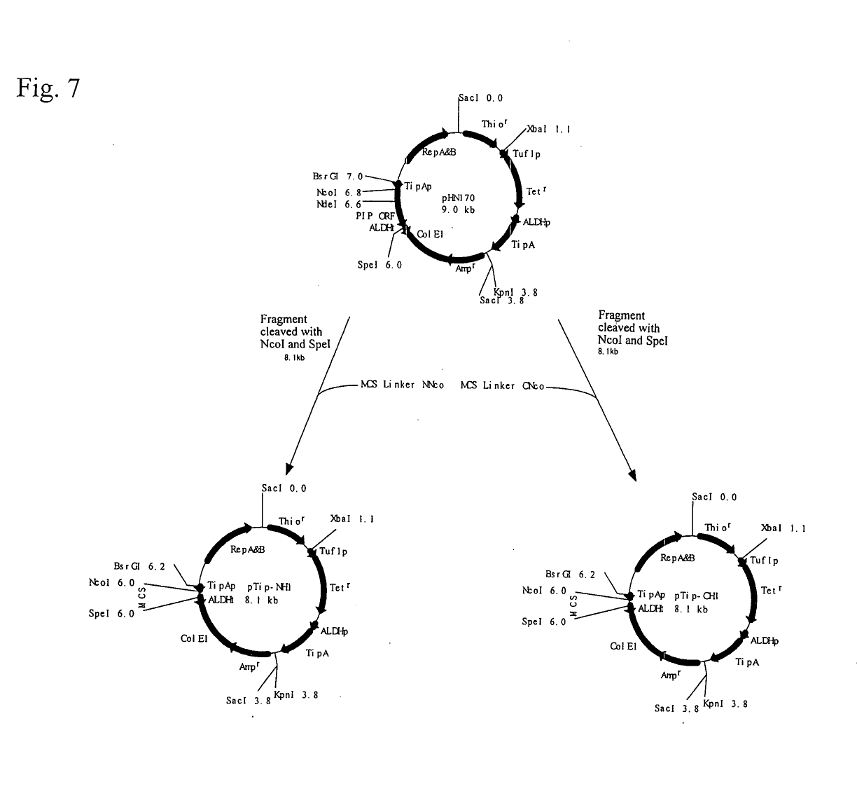

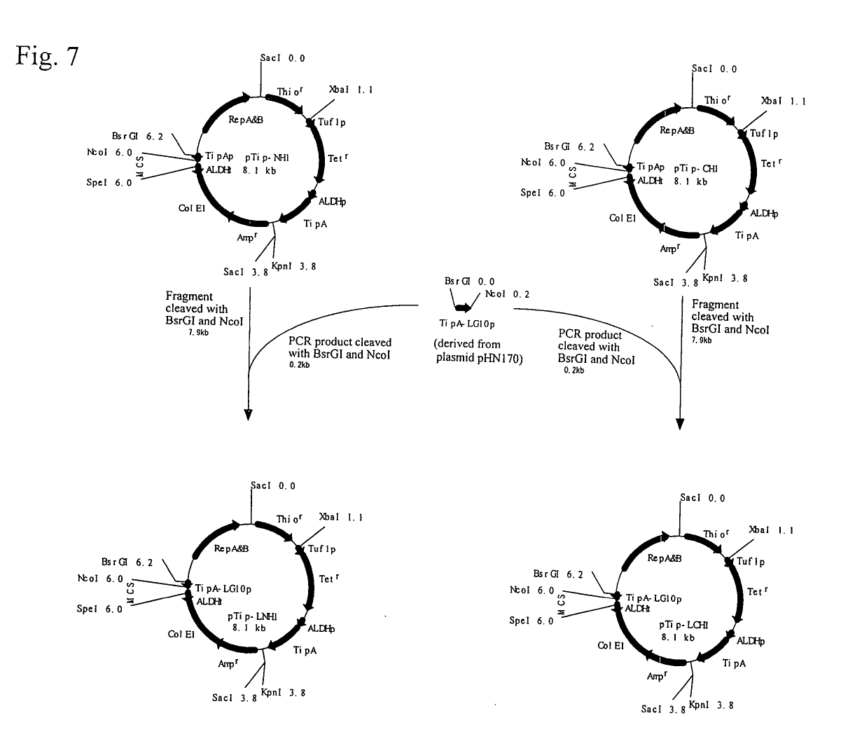

The following procedure was performed in order to remove the PIP gene, which is a reporter, from the plasmid described in Example 7 and to introduce a multicloning site (FIG. 7).

The synthetic oligodeoxyribonucleotides described in SEQ ID Nos. 38 and 39 in the Sequence Listing contain sequences to be multicloning sites and have complementary sequences to each other. These two were mixed in the same molar amount, processed at 70° C. for 10 minutes, and cooled to room temperature for converting them for 20 minutes into a double-strand. As a result, the end parts thereof became linkable to a vector obtained by double digestion by NcoI and SpeI and this double-stranded synthetic DNA (designated as MCS Linker NNco in the drawing) was subcloned into NcoI and SpeI sites of pHN170. The resultant plasmid was named pTip-NH1. Furthermore, the synthetic oligodeoxyribonucleotides described in SEQ ID Nos. 40 and 41 in the Sequence Listing, which contain sequences to be multicloning sites and have complementary sequences to each other, were converted into a double-stranded synthetic DNA in the same way (designated as MCS Linker CNco in the drawing) was subcloned into NcoI and SpeI sites of pHN170. The resultant plasmid was named pTip-CH1.

The hybrid DNA consisting of the TipA gene promoter sequence and the binding site derived from lambda phage gene10 as described in Example 7 was subjected to double digestion by restriction enzymes BsrGI and NcoI, and subcloned into BsrGI and NcoI sites of pTip-NH1 and pTip-CH1, respectively. The resultant plasmids were named pTip-LNH1 and pTip-LCH1.

### EXAMPLE 9

Construction of Vector Plasmid pTip-NH2, pTip-CH2, pTip-LNH2, and pTip-LCH2

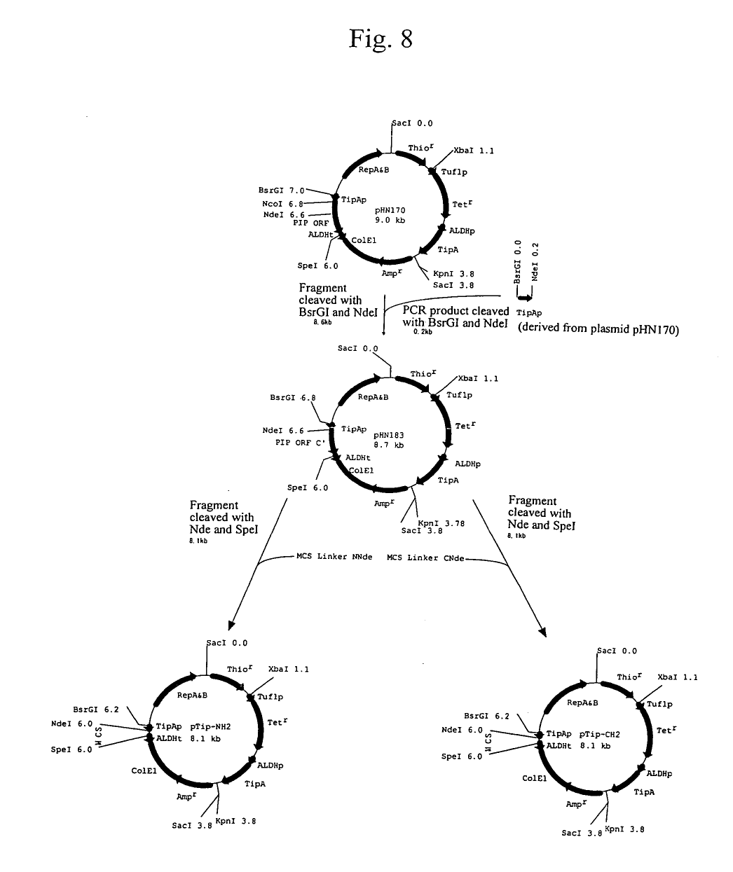

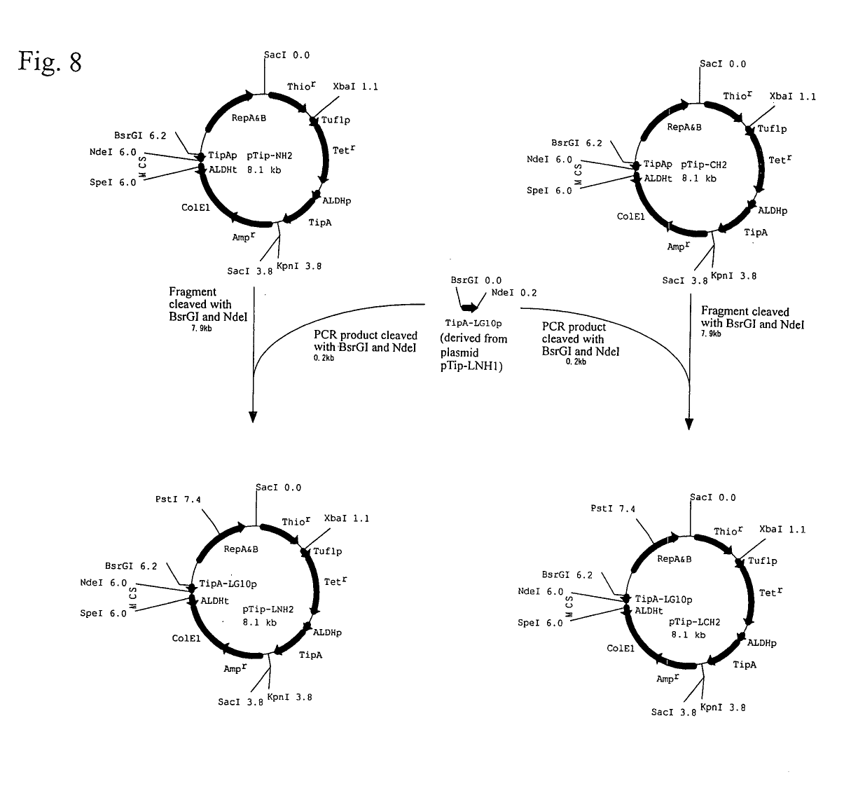

The following procedure was performed to change the NcoI site located most upstream in the multicloning site to NdeI in the plasmids pTip-NH1, pTip-CH1, pTip-LNH1 and pTip-LCH1 described in Example 8 (FIG. 8).

Amplification by PCR was performed using plasmid pHN170 as a template along with primers of SEQ ID Nos. 21 and 42 in the Sequence Listing. Consequently, DNA consisting of the TipA gene promoter was obtained. Double digestion of this DNA fragment of 0.2 kb was carried out with restriction enzymes BsrGI and NdeI, and the product was subcloned into BsrGI and NdeI sites of pHN170. The resultant plasmid was named pHN183.

The synthetic oligodeoxyribonucleotides described in SEQ ID Nos. 43 and 44 in the Sequence Listing contain sequences to be multicloning sites and have complementary sequences to each other. These two were mixed in the same molar amount, processed at 70° C. for 10 minutes, and cooled to room temperature for converting them for 20 minutes into a double-strand. As a result, the end parts thereof became linkable to a vector obtained by double digestion by NdeI and SpeI and this double-stranded synthetic DNA (designated as MCS Linker NNde in the drawing) was subcloned into NdeI and SpeI sites of pHN183. The resultant plasmid was named pTip-NH2. Furthermore, the synthetic oligodeoxyribonucleotides described in SEQ ID Nos. 45 and 46 in the Sequence Listing, which contain sequences to be multicloning sites and have complementary sequences to each other, were converted into a double-stranded synthetic DNA (designated as MCS Linker CNde in the drawing) was subcloned into NdeI and SpeI sites of pHN183. The resultant plasmid was named pTip-CH2.

Amplification by PCR was performed using plasmid pTip-LNH1 as a template along with primers of SEQ ID Nos. 21 and 47 in the Sequence Listing. Consequently, a hybrid DNA consisting of the TipA gene promoter and the binding site derived from lambda phage gene10 was obtained. Double digestion of this DNA fragment of 0.2 kb was carried out with restriction enzymes BsrGI and NdeI, and the product was subcloned into BsrGI and NdeI sites of pHN170 sites of pTip-NH2 and pTip-CH2, respectively. The resultant plasmids were named pTip-LNH2 and pTip-LCH2, respectively.

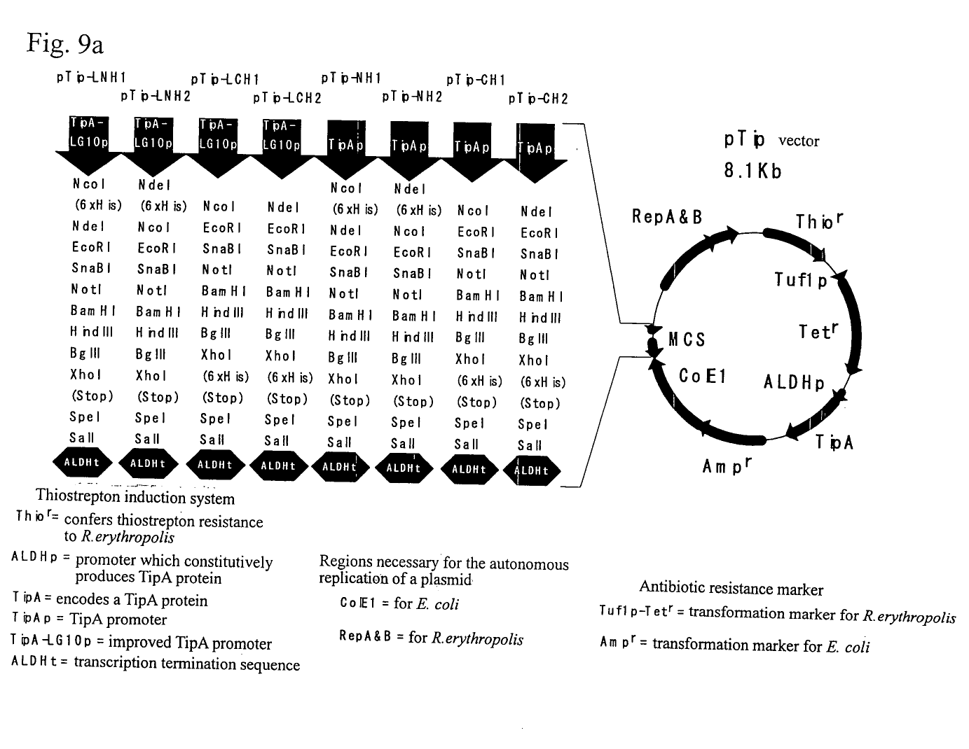

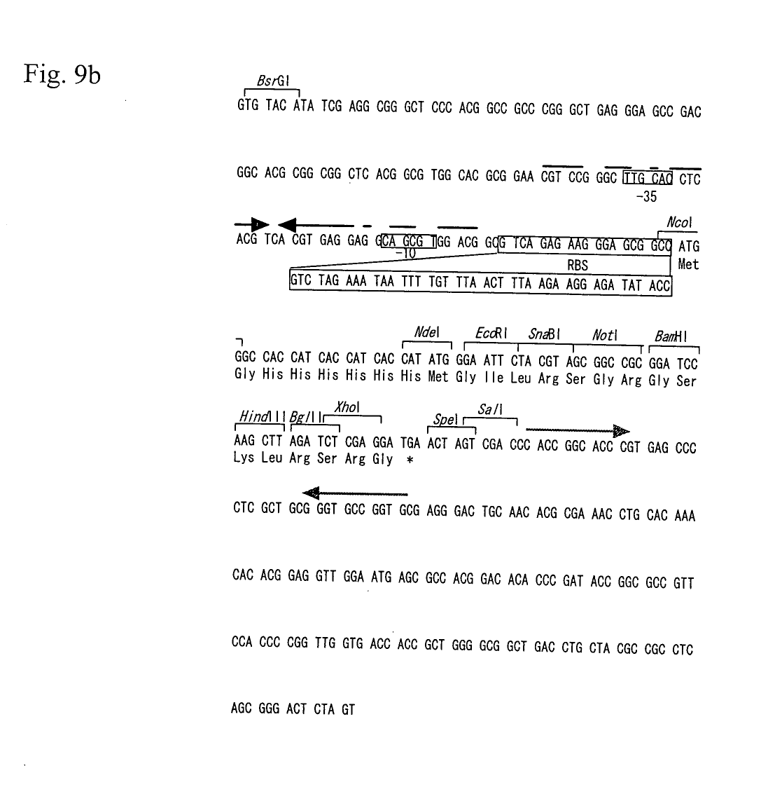

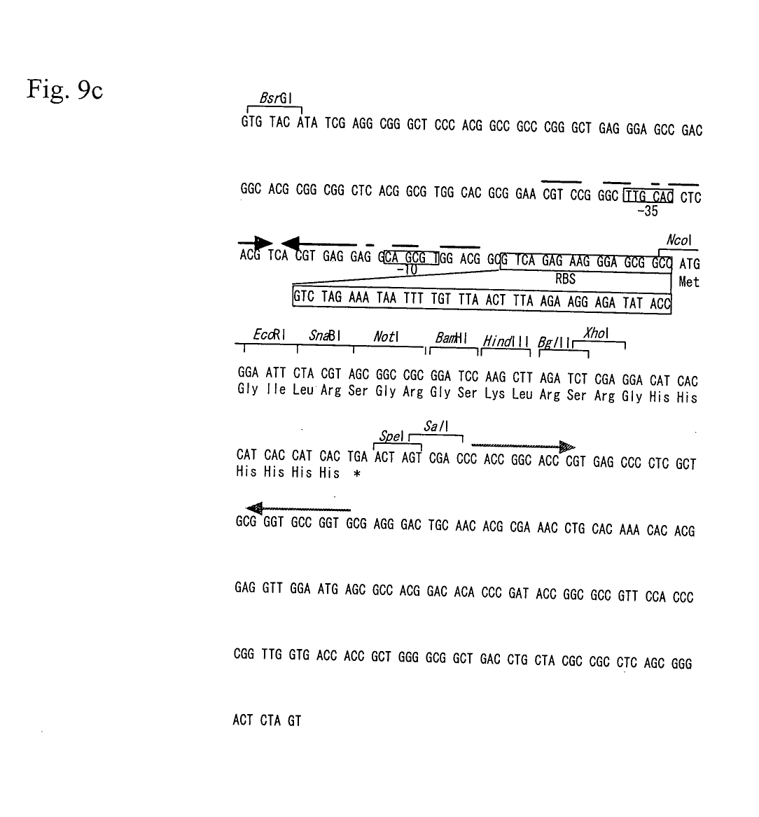

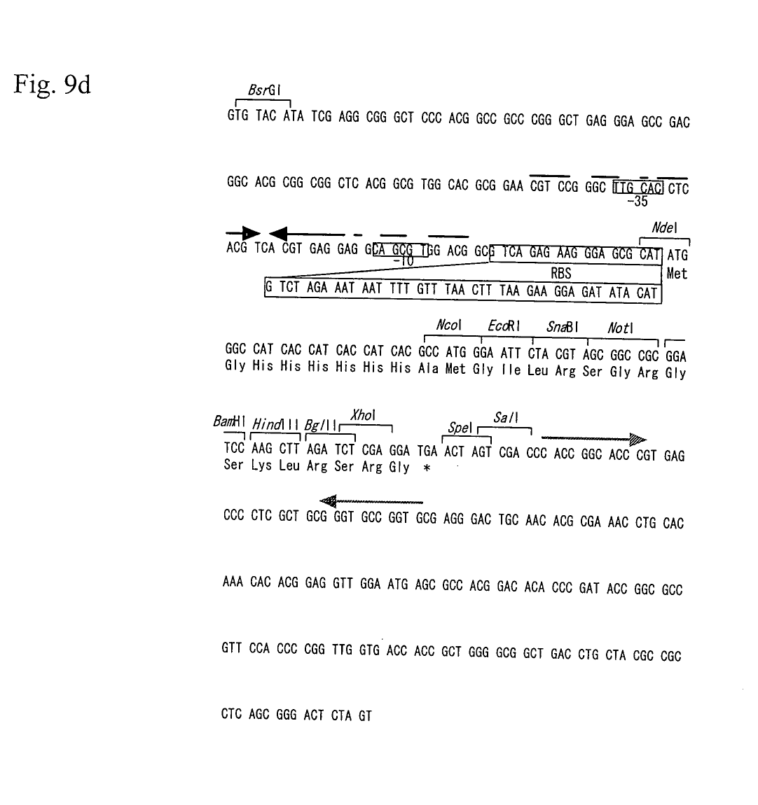

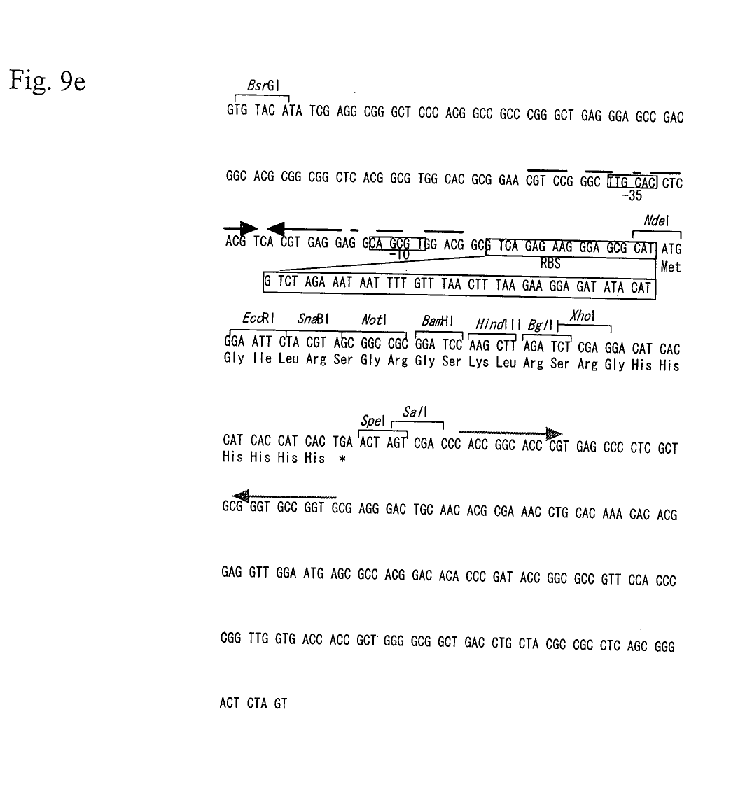

The map of the plasmids created in Examples 8 and 9 and the sequence around the multicloning site are summarized in FIG. 9. In the drawing, the solid arrow indicates an inverted repeat sequence which exists in the TipA gene promoter. The hatched arrow indicates an inverted repeat sequence which exists in the transcription termination sequence of ThcA gene. RBS, −10 region and −35 region generally present in the promoter domain of a procaryote and important for transcription of a gene, are boxed. In addition, the most important SD sequence (Shine and Dalgarno, Eur. J. Biochem. 57 221-230 (1975)) in RBS is underlined. The thiostrepton induction system in FIG. 9a contains Thio^{r}, ALDHp, TipA, TipAp, TipA-LG10p and ALDHt. Thio^{r} confers thiostrepton resistance to R. erythropolis. ALDHp indicates a promoter which constitutively produces TipA protein, and TipA encodes a TipA protein. In addition, TipAp indicates a TipA promoter, TipA-LG10 p indicates an improved TipA promoter, and ALDHt indicates the transcription termination sequence. Furthermore, ColE1 and RepA&B are contained as regions necessary for the autonomous replication of a plasmid. ColE1 is for E. coli, and RepA&B is for R. erythropolis. Furthermore, Tuflp-Tet^{r} and Ampr are contained as an antibiotic resistance marker. Tuf1 p-Tet^{r} is a transformation marker for R. erythropolis and Amp^{r} is a transformation marker for E. coli.

### EXAMPLE 10

Construction of Vector Plasmid pTip-CH1.1, pTip-CH2.1, pTip-LCH1.1 and pTip-LCH2.1

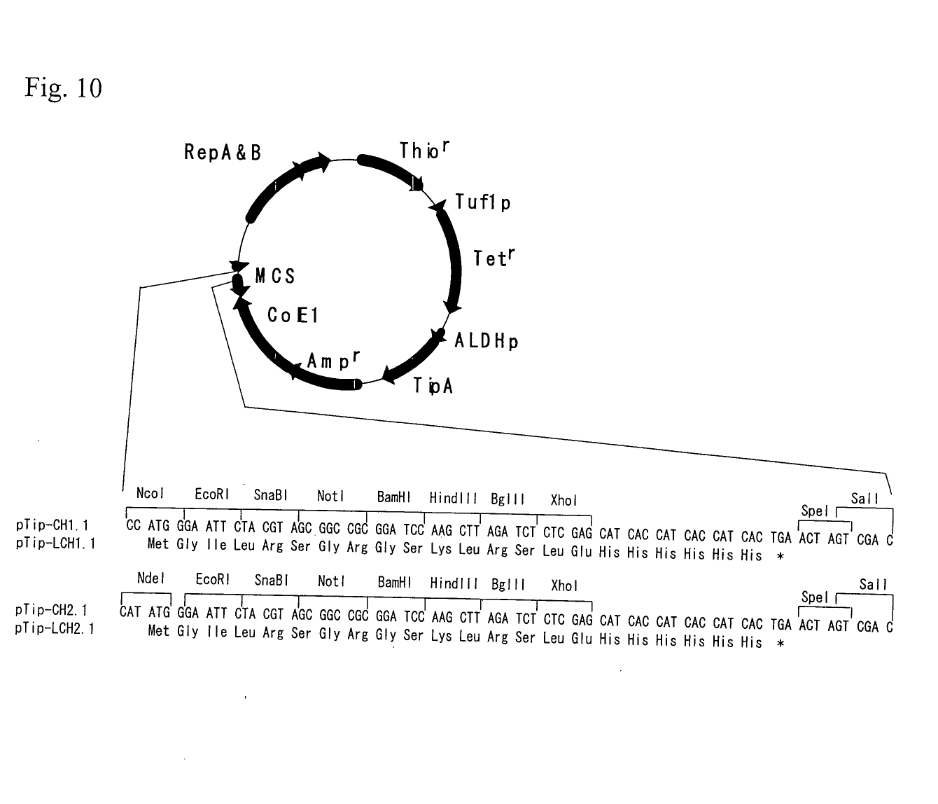

The following procedure was performed in order to make the reading frame after the XhoI site of the multicloning site to agree with the reading frame of a commercial pET vector (Novagen) in the plasmids pTip-CH1, pTip-CH2, pTip-LCH1 and pTip-LCH2 described in Examples 8 and 9 (FIG. 10).

Amplification by PCR was performed using plasmid pTip-CH1 as a template along with primers of SEQ ID Nos. 21 and 104 in the Sequence Listing. Consequently, DNA containing the TipA gene promoter and a multicloning site was obtained. Double digestion of this DNA fragment of 0.3 kb was carried out with restriction enzymes BsrGI and SpeI, and the product was subcloned into BsrGI and SpeI sites of pTip-CH1. The resultant plasmid was named pTip-CH1.1.

Amplification by PCR was performed using plasmid pTip-CH2 as a template along with primers of SEQ ID Nos. 21 and 104 in the Sequence Listing. Consequently, DNA containing the TipA gene promoter and a multicloning site was obtained. Double digestion of this DNA fragment of 0.3 kb was carried out with restriction enzymes BsrGI and SpeI, and the product was subcloned into BsrGI and SpeI sites of pTip-CH1. The resultant plasmid was named pTip-CH2.1.

Amplification by PCR was performed using plasmid pTip-LCH1 as a template along with primers of SEQ ID Nos. 21 and 104 in the Sequence Listing. Consequently, DNA containing the TipA-LG10 promoter and a multicloning site was obtained. Double digestion of this DNA fragment of 0.3 kb was carried out with restriction enzymes BsrGI and SpeI, and the product was subcloned into BsrGI and SpeI sites of pTip-CH1. The resultant plasmid was named pTip-LCH1.1.

Amplification by PCR was performed using plasmid pTip-LCH2 as a template along with primers of SEQ ID Nos. 21 and 104 in the Sequence Listing. Consequently, DNA containing the TipA-LG10 promoter and a multicloning site was obtained. Double digestion of this DNA fragment of 0.3 kb was carried out with restriction enzymes BsrGI and SpeI, and the product was subcloned into BsrGI and SpeI sites of pTip-CH1. The resultant plasmid was named pTip-LCH2.1.

### EXAMPLE 11

Construction of Vector Plasmids pHN172 and pHN173

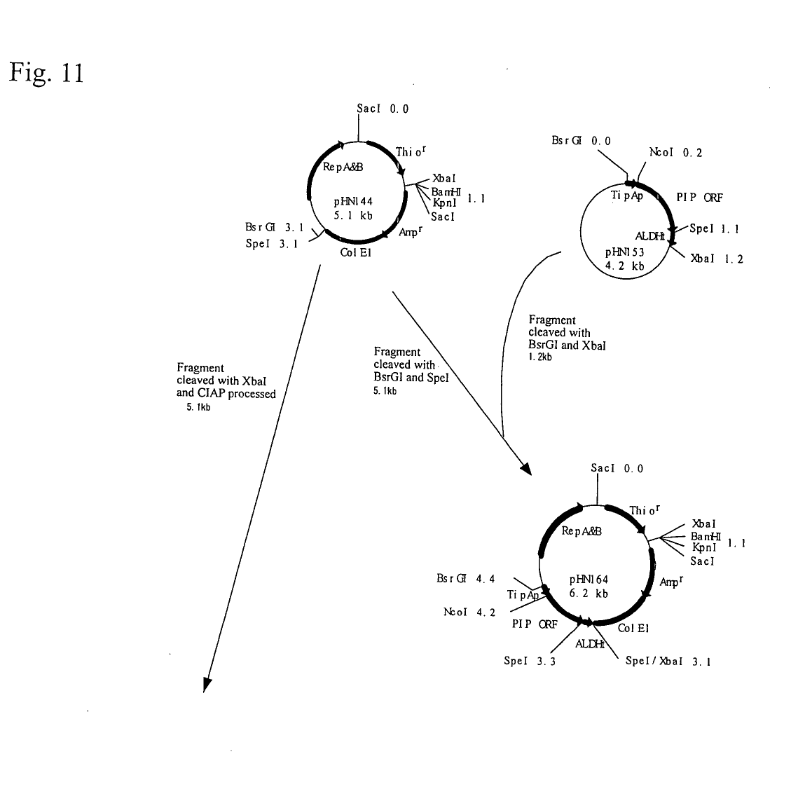

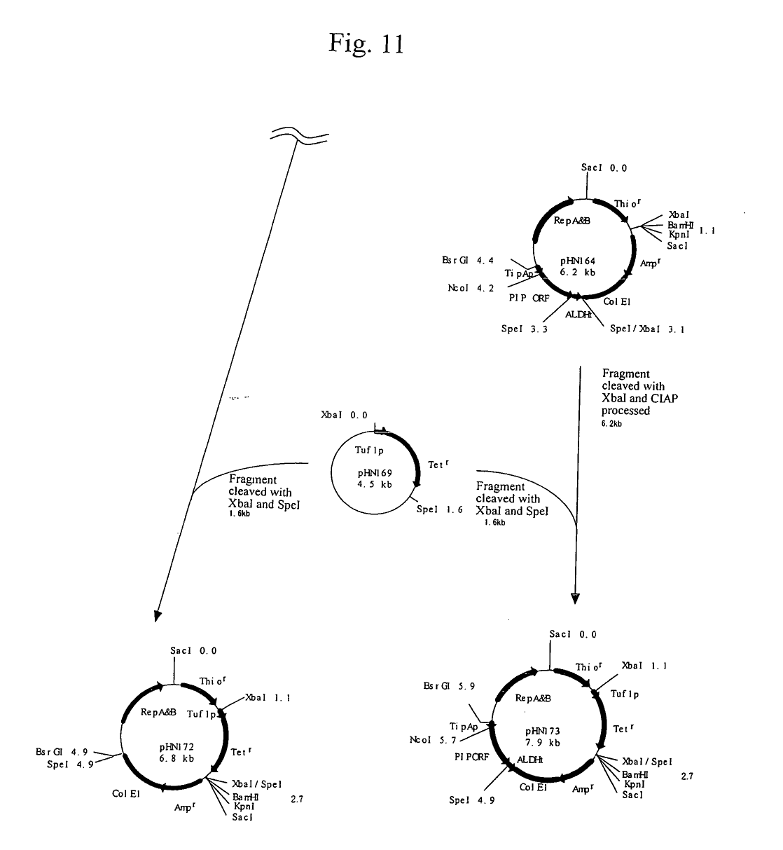

In order to investigate whether the induction of expression is strictly regulated, the following plasmids for control experiment were created (FIG. 11).

A DNA fragment of 1.6 kb obtained by double digestion of pHN169 by XbaI and SpeI was subcloned into XbaI site of plasmid pHN144 (the direction of subcloning was tsr gene ORF-tetracycline resistance gene ORF-ampicillin resistance gene ORF from 5′ end of the DNA). The resultant plasmid was named pHN172.

Next, a DNA fragment of 1.2 kb obtained by double digestion of pHN153 by BsrGI and XbaI was subcloned into BsrGI and SpeI sites of plasmid pHN144. The resultant plasmid was named pHN164. Subsequently, a DNA fragment of 1.6 kb obtained by double digestion of pHN164 by XbaI and SpeI was subcloned into XbaI site of plasmid pHN164(the direction of subcloning was tsr gene ORF-tetracycline resistance gene ORF-ampicillin resistance gene ORF from 5′ end of the DNA). The resultant plasmid was named pHN173.

pHN170 has both of a gene cassette having three elements of the TipA gene promoter, PIP ORF linked downstream thereof and the ThcA transcription termination sequence further downstream thereof (hereinafter referred to as expression cassette) and a gene cassette having two elements of the ThcA gene promoter and the TipA gene downstream thereof (hereinafter referred to as inducer cassette). pHN173 has only expression cassette and pHN172 does not have either of the cassettes.

### EXAMPLE 12

Transformation of a Bacterium of Genus Rhodococcus

Rhodococcus erythropolis JCM 3201 strain is subjected to shaking culture in 100 ml of LB culture media at 30° C. until the logarithmic growth phase. The culture solution is ice-cooled for 30 minutes and centrifuged to collect cell bodies. 100 ml of ice-cooled sterilized water is added to this, agitated well and centrifuged again to collect cell bodies. 100 ml of ice-cooled 10% glycerin solution is added to this, agitated well and centrifuged to collect cell bodies. Washing with ice-cooled 10% glycerin solution is repeated once again, and the cell bodies are suspended in 5 ml of ice-cooled 10% glycerin solution. The solution is dispensed into 400 μl aliquots, instantly frozen by liquid nitrogen and it stored at −80° C. until it is used. The cell bodies were taken out from −80° C., thawed on ice, and a plasmid of pHN170 or pHN172 or pHN173 was added in an amount of 3 μl (respectively about 300 ng). This mixed solution of cell bodies and DNA is transferred to an electroporation cuvette (Bio-Rad: 0.2 cm gap cuvette) and electrically pulsed by using Gene Pulsar II, gene introduction equipment of the same company with the electric field of 12.5 kV/cm, under setup conditions of the pulse controller at 25 μF of capacitance and 400 Ω of external resistance, respectively. The mixed solution of cell bodies and DNA subjected to electric pulsed processing was mixed to 1 ml of LB culture medium, and after cultured at 30° C. for 4 hours, the cell bodies were collected and applied to LB agar culture medium containing 20 μg/ml of tetracycline (agar concentration: 1.8%), and cultured at 30° C. for 3 days to obtain each of the transformants.

### EXAMPLE 13

Measurement 1 of the PIP Activity in a Bacterium of Genus Rhodococcus

The constructed expression vector contains PIP gene as a reporter gene, and the inductivity by thiostrepton and the strength of induction etc. can be confirmed by measuring the enzyme activity of PIP. The amount of PIP which exists in the cell bodies can be easily quantified by observing the hydrolyzation activity on an artificial substrate H-Pro-β NA (product of Bachem).

The transformant of Rhodococcus erythropolis JCM3201 strain created in Example 12 is cultured at 30° C. in an LB culture medium containing 8 μg/ml of tetracycline, and when the optical density (O.D.600) measured at the wavelength of 600 nm reached 0.6, thiostrepton (solvent is dimethylsulfoxide) was added so that the final concentration of thiostrepton might be 1 μg/ml thereby inducing the expression of PIP.

A part of culture solution is taken out in 16 hours, the volume is raised to total 200 μl by LB culture medium containing 8 1g/ml tetracycline and warmed at 60° C. for 1 minute. 2 μl of H-Pro-β NA (100 mM: solvent dimethylsulfoxide) is added thereto as a substrate of PIP and incubated at 60° C. for 20 minutes (60° C. is optimal temperature of PIP). In order to observe free β NA hydrolyzed from H-Pro-β NA by PIP, 134 μl of Fast Garnet GBC Salt solution (product of Wako Pure Chemicals Company; concentration: 0.5 mg/ml; solvent: 1 M acetic acid sodium buffer (pH 4.2) and 10% Triton X-100) is added as a coloring agent. The above-mentioned mixed solution exhibits yellow color when PIP is not expressed, while exhibiting red color when PIP is expressed. The exhibited red color was measured by measuring absorbance at 550 nm (A550) using a spectrophotometer to quantify the PIP activity. The measurement was performed by adding and diluting 666 μl of sterilized water after adding FastGarnet GBC Salt.

Since the optical density of the cell is also measured at 550 nm in this measurement, the optical density of the cell at 550 nm (O.D.550) is separately measured, and the value equivalent to O.D.550 used at the time of measurement is subtracted from the value of A550 to obtain a corrected value Ac550. That is, Ac550 can be calculated by:

Ac550=A550−O.D.550×(amount (ml) of culture solution used for measuring PIP activity). The unit value was defined as “the value of Ac550 obtained by the measurement for 20 minutes per 1 ml of culture solution per O.D. 600=1” calculated by “Ac550/(amount (ml) of culture solution used for measuring PIP activity)/O.D.600.”

The cells transformed by pHN170 exhibited yellow color when they were cultured without adding thiostrepton, whereas they exhibited red color when thiostrepton was added. As for the cells transformed by pHN172 and pHN173, they exhibited yellow color either in the presence or absence of thiostrepton.

The transformant of Rhodococcus erythropolis JCM3201 strain created in Example 12 is cultured at 30° C. in an LB culture medium containing 8 μg/ml of tetracycline, and when O.D.600 reached 2.0, the medium was cooled to a temperature of 4° C. immediately and cultured with shaking for one hour in order to make cell bodies naturalized. Thiostrepton was added so that the final concentration thereof might be 1 μg/ml to induce the expression of PIP. A part of culture solution was taken out in 40 hours, and the same experiment as the above-mentioned was conducted at 30° C.

The cells transformed by pHN170 exhibited yellow color when they were cultured without adding thiostrepton, whereas they exhibited red color when thiostrepton was added. As for the cells transformed by pHN172 and pHN173, they exhibited yellow color either in the presence or absence of thiostrepton.

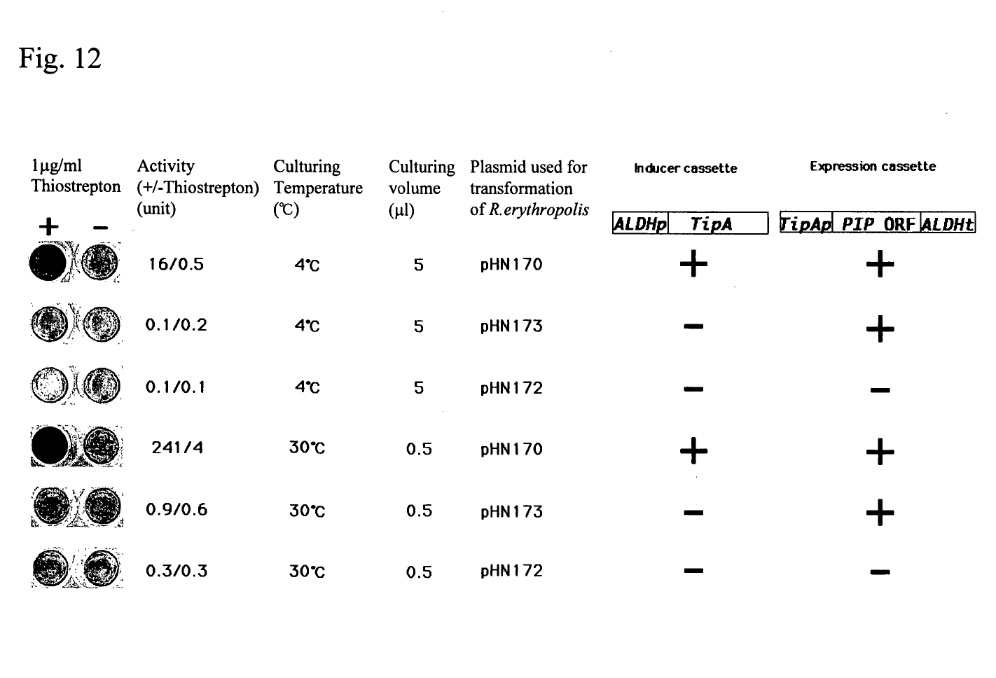

The above result is summarized in FIG. 12.

As shown in FIG. 12, Rhodococcus erythropolis JCM3201 strain was transformed by pHN170, pHN172 and pHN173, and PIP activity was measured respectively for the cases where PIP is expressed or not expressed at 30° C. or 4° C. Whether the thiostrepton is added (+) or not (−) with a final concentration of 1 μg/ml, activity value, culturing temperature, amount of culturing solution, transformed plasmid and whether the plasmid has “cassette” (+) or not (−) are shown in FIG. 12.

It was confirmed from these results that the expression vector which can be induced by thiostrepton in the wide temperature range had been constructed.

### EXAMPLE 14

Measurement 2 of the PIP Activity in a Bacterium of Genus Rhodococcus

The PIP activity of the cells transformed from Rhodococcus erythropolis JCM3201 strain by pHN170 and pHN171 created in Example 12 were measured in the same manner as in Example 13.

The results of measuring PIP activity with time after adding thiostrepton with a final concentration of 1 μg/ml are shown in FIG. 13. This figure shows the activity with time of Rhodococcus erythropolis JCM3201 strain transformed by pHN170, and made to express PIP at 30° C. and 4° C. In FIG. 13, the vertical axis indicates the activity value (unit) of PIP and the horizontal axis indicates the time after thiostrepton with a final concentration of 1 μg/ml is added. The symbol “O” at 4° C. shows activity when O.D.600 is 1.0 and an expression induction is started, and the symbol “□” shows activity when O.D.600 is 2.0 and an expression induction is started. The symbol “O” at 30° C. shows activity when O.D.600 is 0.6 and an expression induction is started, and the symbol “□” shows activity when O.D.600 is 1.0 and an expression induction is started.

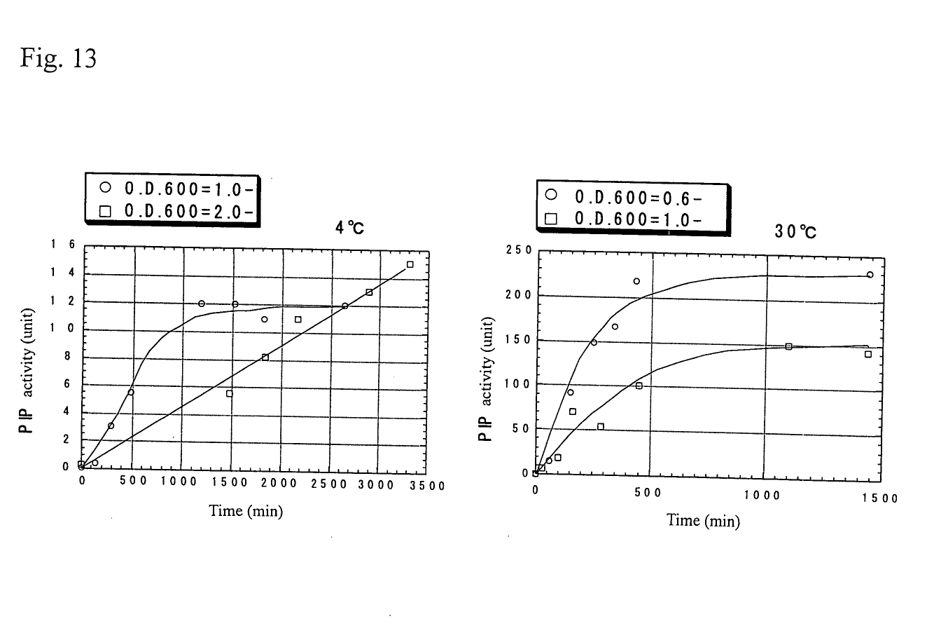

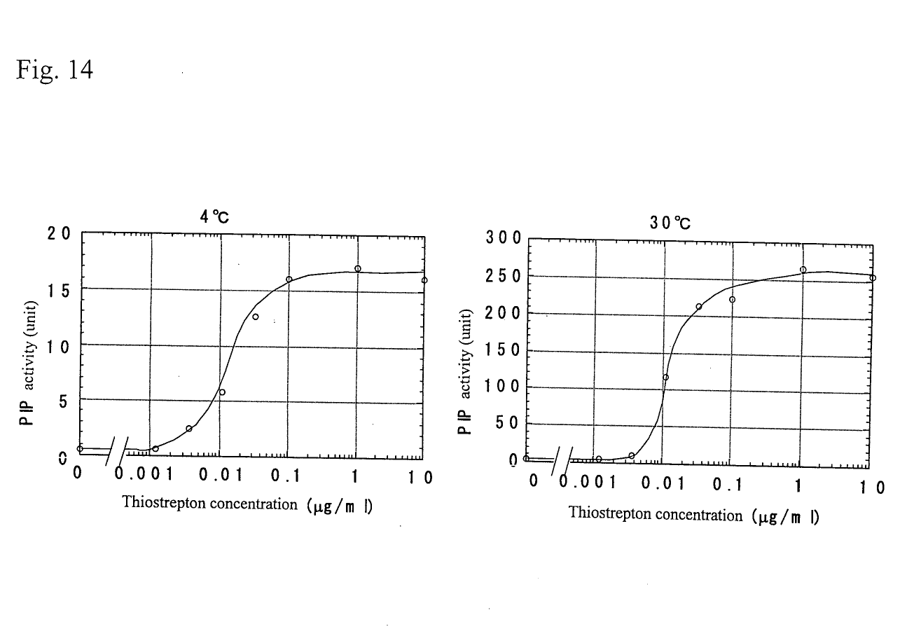

FIG. 14 shows the results in the case where the final concentration of thiostrepton added was varied. In this case, induction expression time was 2400 minutes (40 hours) in the case where induction was started at 4° C. when O.D.600=2.0, and 960 minutes (16 hours) in the case where induction was started at 30° C. when O.D.600=0.6. Examples shown in FIG. 14 show the PIP activity of the cells transformed from Rhodococcus erythropolis JCM3201 strain by pHN170 measured at various concentrations of thiostrepton added to express PIP at 30° C. and 4° C. In FIG. 14, the vertical axis indicates the activity value (unit) of PIP and the horizontal axis indicates the final concentration (μg/ml) of thiostrepton added in the culture medium.

These results have revealed that 1 μg/ml of thiostrepton is enough for expression induction either at 30° C. or 4° C. Moreover, although depending on the timing of expression induction, the amount of expression of PIP per cell reaches the maximum for 500 to 1000 minute (about 8 to 16 hours) as for the case of 30° C., and 3000 minutes (50 hours) or more as for the case of 4° C.

### EXAMPLE 15

Measurement 3 of the PIP Activity in a Bacterium of Genus Rhodococcus

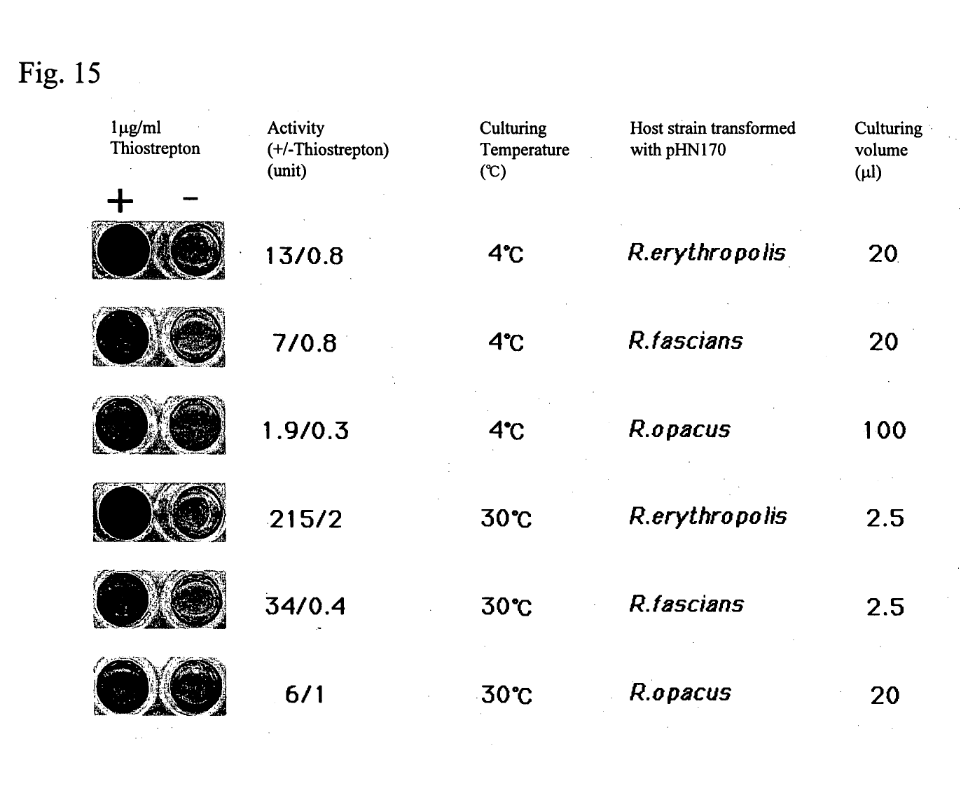

Rhodococcus erythropolis JCM3201 strain, Rhodococcus fascians JCM10002 strain and Rhodococcus opacus DSM44193 strain are transformed by pHN170 in the same manner as in Example 12. Consequently, the transformation not only of Rhodococcus erythropolis but also Rhodococcus fascians and Rhodococcus opacus was able to be carried out by pHN170. Therefore, it was shown that the autonomous replication starting point derived from Rhodococcus erythropolis JCM2895 strain introduced into pHN170 functions also in Rhodococcus fascians and Rhodococcus opacus. The PIP activity was measured using these transformants in the same manner as in Example 13. In each of the strains, induction expression time was 2400 minutes (40 hours) in the case where induction was started at 4° C. when O.D.600=2.0, and 960 minutes (16 hours) in the case where induction was started at 30° C. when O.D.600=0.6. The results are shown in FIG. 15. In FIG. 15, the PIP activity was measured for the cells transformed from Rhodococcus erythropolis JCM3201 strain, Rhodococcus fascians JCM10002 strain and Rhodococcus opacus DSM44193 strain by pHN170 and for each case where PIP is expressed or not expressed at 30° C. or 4° C. Whether the thiostrepton is added (+) or not (−) with a final concentration of 1 μg/ml, activity value, culturing temperature, amount of culturing solution, transformed plasmid and the hosts transformed by pHN170 are shown in FIG. 15.

Every bacterium of genus Rhodococcus transformed by pHN170 exhibited yellow color when they were cultured without adding thiostrepton, whereas they exhibited red color when thiostrepton was added. However, in Rhodococcus fascians JCM10002 strain and Rhodococcus opacus DSM44193 strain, expression was low compared with Rhodococcus erythropolis JCM3201 strain.

### EXAMPLE 16

Expression and Purification 1 of a Foreign Protein in a Bacterium of Genus Rhodococcus

The transformation of Rhodococcus erythropolis JCM3201 strain was carried out in the same manner as in Example 12, and PIP was made to express at 30° C. and 4° C. in the similar manner as in Example 13 using pHN170 (described in Example 7) and pHN171 (described in Example 7), respectively. Here, after adding thiostrepton with a final concentration of 1 μg/ml, cell bodies were collected with time and PIP was purified. The PIP has 6× His tag linked at C-terminal thereof, and purified using nickel-NTA Superflow (product of Qiagen) following the manufacturer's instructions.

The purification process was specifically described below, where the procedure of purification was performed at 4° C. The cell bodies which expressed protein (a part for 20 ml culture solution) were collected, and suspended into 1 ml of NT-Buffer (50 mM Tris-HCl (pH 8.0), 100 mM sodium chloride, 1 mM dithiothreitol), and 1 g of glass beads (diameter of 0.105-0.125 mm) was added. The cells were destroyed by reciprocally shaking by Fast-prep FP120 (product of SAVANT) with 6 m/second in speed for 20 seconds. After centrifuged in 20,000×g, nickel-NTA Superflow which was beforehand equilibrated with NT-Buffer was added to 700 μl of the supernatant so that the bed volume might be 40 μl. This mixture was subjected to rotation agitation for 1 hour to combine the nickel-NTA Superflow beads to the protein attached with 6× His tag. After washing these beads 4 times by NT-Buffer, the protein was eluted from the protein attached with 6× His tag by suspending these beads in 120 μl of NTE-Buffer (50 mM Tris-HCl (pH 7.0), 100 mM sodium chloride, 1 mM dithiothreitol, 400 mM imidazole) 3 times.

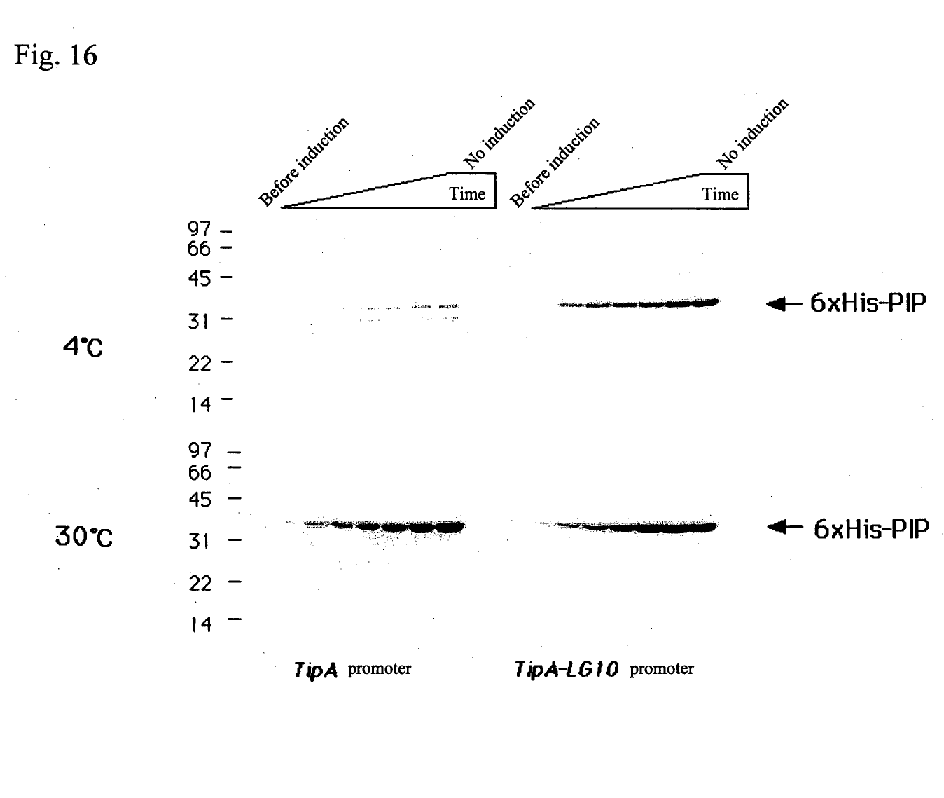

The results where 10 μl of the above-mentioned samples was subjected to 12% of SDS polyacrylamide electrophoresis according to an ordinary method are shown in FIG. 16. The transformation of Rhodococcus erythropolis JCM3201 strain was carried out by pHN170 (expression from the TipA gene promoter: two panels on the left), and pHN171 (expression from the TipA-LG10 promoter: two panels on the right), and PIP was made to express at 4° C. (the upper two panels) and 30° C. (lower two panels). After adding thiostrepton with the final concentration of 1 μg/ml, cell bodies were collected with time and purified using nickel-NTA Superflow using the 6× His tag attached to C-terminal of PIP. The timing for collecting cell bodies is set 0 minute (leftmost lane), 180 minutes (second lane from the left), 420 minutes (third lane from the left), 1080 minutes (fourth lane from the left), 1440 minutes (fifth lane from the left), 1860 minutes (sixth lane from the left), 2520 minutes (seventh lane from the left) and 3060 minutes (eight lane from the left) at 4° C. and 0 minute (leftmost lane), 120 minutes (second lane from the left), They are 240 minutes (third lane from the left), 420 minutes (fourth lane from the left), 540 minutes (fifth lane from the left), 720 minutes (sixth lane from the left), 900 minutes (seventh lane from the left) and 1440 minutes (eight lane from the left) at 30° C. In every panel in FIG. 16, the rightmost lane shows the sample purified from cell bodies which continued culturing without inducing (namely, without adding thiostrepton). At 30° C., expression from the TipA-LG10 promoter was a little lower compared with the expression from the TipA gene promoter, whereas at 4° C., expression from the TipA-LG10 promoter was conversely higher. In addition, the induction of expression was controlled strictly also in TipA-LG10 promoter.

Detailed comparison in the amount of expression from both the promoters are described in Example 18 in detail.

### EXAMPLE 17

Expression and Purification 2 of a Foreign Protein in a Bacterium of Genus Rhodococcus

The transformation of Rhodococcus erythropolis JCM3201 strain was carried out in the same manner as in Example 12, and PIP was made to express in the similar manner as in Example 13 using pHN170 (described in Example 7), and pHN171 (described in Example 7), respectively at 32° C., 30° C., 15° C., and 4° C. Induction expression time was 2400 minutes (40 hours) in the case where induction was started at 4° C. when O.D.600=2.0, 1500 minutes (25 hours) in the case where induction was started at 15° C. when O.D.600=1.0, 960 minutes (16 hours) in the case where induction was started at 30° C. when O.D.600=0.6, and 960 minutes (16 hours) in the case where induction was started at 32° C. when O.D.600=0.6. The thiostrepton added is in an amount of 1 μg/ml by the final concentration. Purification was performed in the same manner as Example 16.

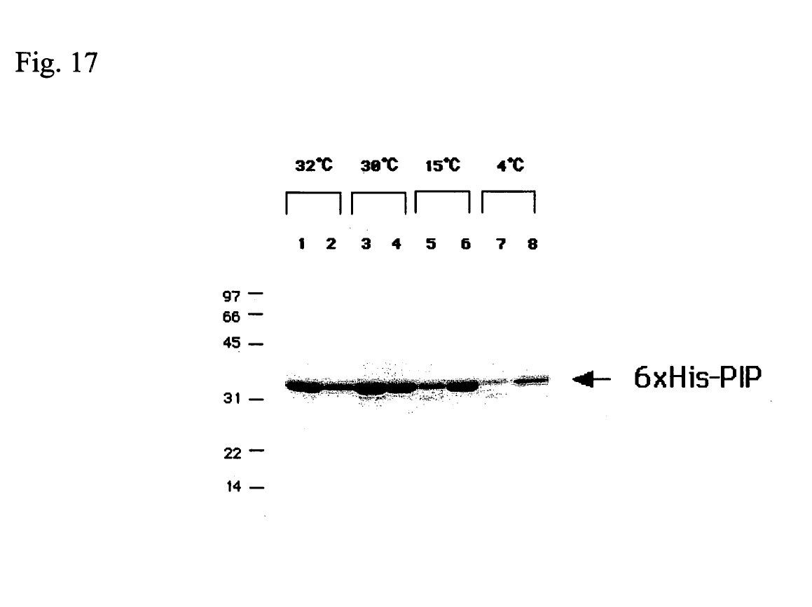

The results where 10 μl of the above-mentioned samples was subjected to 12% of SDS polyacrylamide electrophoresis according to an ordinary method are shown in FIG. 17. The transformation of Rhodococcus erythropolis JCM3201 strain was carried out by pHN170 (expression from the TipA gene promoter: lanes 1, 3, 5 and 7), and pHN171 (expression from the TipA-LG10 promoter: lanes 2, 4, 6 and 8), and PIP was made to express at 4° C. (lanes 7 and 8), 15° C. (lanes 5 and 6), 30° C (lanes 3 and 4) and 32° C. (lanes 1 and 2). Purification was performed using nickel-NTA Superflow utilizing the 6× His tag linked to C-terminal of PIP.

In the wide temperature range from 32 to 4° C., expression of PIP from the TipA gene promoter and TipA-LG10 promoter was confirmed. The amounts of expression of PIP were higher from the TipA promoter at 32° C. and 30° C., whereas the amounts of expression were higher from TipA-LG10 promoter at 15° C. and 4° C.

### EXAMPLE 18

Expression and Purification 3 of Foreign Proteins in a Bacterium of Genus Rhodococcus

The following experiments were conducted in order to investigate whether a protein other than PIP can also be made to express using this expression vector.

Amplification by PCR was performed using plasmid pRSET-ATPIP as a template along with primers of SEQ ID Nos. 48 and 49 in the Sequence Listing. Consequently, DNA containing PIP gene derived from Arabidopsis thaliana (Tamura et al., FEBS Lett. 398 101-105 (1996)—hereinafter abbreviated as AtPIP) was obtained. Double digestion of this DNA fragment of 1.0 kb was carried out with restriction enzymes NcoI and XhoI, and the product was subcloned into NcoI and XhoI sites of pTip-CH1 and pTip-LCH1. As a result, plasmids containing AtPIP gene under control of the TipA gene promoter TipA-LG10 promoter were created, and the resultant plasmids were named pHN176 and pHN177, respectively.

Amplification by PCR was performed using plasmid pTrc99a-GFP as a template along with primers of SEQ ID Nos. 50 and 51 in the Sequence Listing. Consequently, DNA containing a gene encoding fluorescence green protein derived from Aequorea Victoria (hereinafter abbreviated as GFP) was obtained. Double digestion of this DNA fragment of 0.8 kb was carried out with restriction enzymes NcoI and SnaBI, and the product was subcloned into Nco1 and SnaB1 sites of pTip-NH1 and pTip-LNH1. As a result, plasmids containing GFP (having a 6× His tag linked to N-terminal) gene under control of the TipA gene promoter or TipA-LG10 promoter were created, and the resultant plasmids were named pHN187 and pHN186, respectively.

Amplification by PCR was performed using plasmid pGEX-2T (Amersham Bioscience Company) as a template along with primers of SEQ ID Nos. 52 and 53 in the Sequence Listing. Consequently, DNA containing a gene encoding glutathione-S-transferase protein (hereinafter abbreviated as GST) was obtained. Double digestion of this DNA fragment of 0.7 kb was carried out with restriction enzymes NcoI and XhoI, and the product was subcloned into NcoI and XhoI sites of pTip-NH2 and pTip-LNH2. As a result, plasmids containing GST (having a 6× His tag linked to N-terminal) gene under control of the TipA gene promoter or TipA-LG10 promoter were created, and the resultant plasmids were named pHN282 and pHN283, respectively.

The transformation of Rhodococcus erythropolis JCM3201 strain was carried out in the same manner as in Example 12 using pHN170 (described in Example 7), pHN171 (described in Example 7), pHN176, pHN177, pHN187, pHN186, pHN282 and pHN283, and each of the proteins PIP, AtPIP, GFP, and GST was made to express in the similar manner as in Example 13 respectively at 30° C. and 4° C. Induction expression time was 2400 minutes (40 hours) in the case where induction was started at 4° C. when O.D.600=2.0, 960 minutes (16 hours) in the case where induction was started at 30° C. when O.D.600=0.6. The thiostrepton added is in an amount of 1 μg/ml by the final concentration. Purification was performed from 50 ml of culture solution at 4° C. and from 20 ml of culture solution at 30° C.

All of the four above-mentioned proteins have a 6× His tag and purification was performed in the same manner as Example 16.

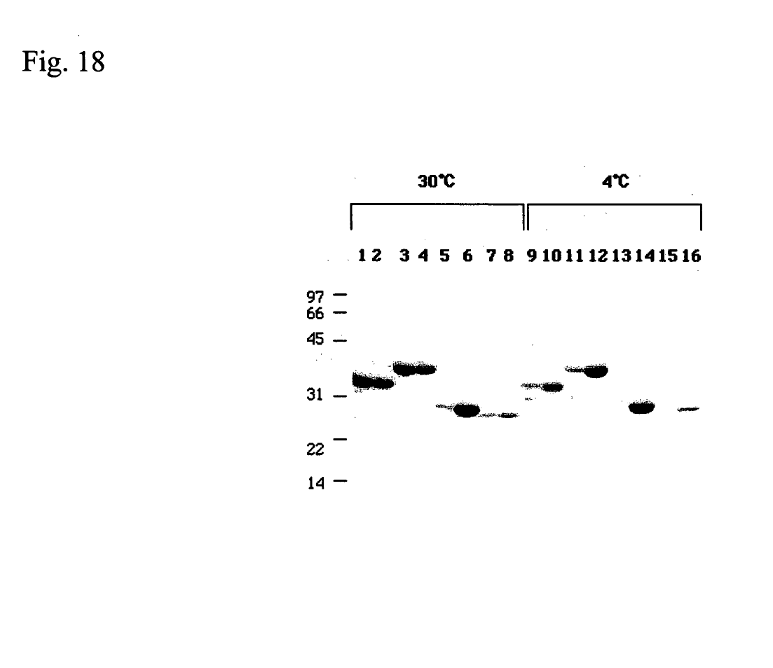

The results where 10 μl of the above-mentioned samples was subjected to 12% of SDS polyacrylamide electrophoresis according to an ordinary method are shown in FIG. 18. The transformation of Rhodococcus erythropolis JCM3201 strain was carried out by pHN170 (PIP located downstream of the TipA gene promoter: lanes 1 and 9), pHN171 (PIP located downstream of the TipA-LG10 promoter: lanes 2 and 10), pHN176 (AtPIP located downstream of the TipA gene promoter: lanes 3 and 11), pHN177 (AtPIP located downstream of the TipA-LG10 promoter: lanes 4 and 12), pHN187 (GFP located downstream of the TipA gene promoter: lanes 5 and 13), pHN186 (GFP located downstream of the TipA-LG10 promoter: lanes 6 and 14), pHN282 (GST located downstream of the TipA gene promoter: lanes 7 and 15) and pHN283 (GST located downstream of the TipA-LG10 promoter: lanes 8 and 16) and the proteins were made to express at 4° C. (lanes 9 to 16) and 30° C. (lanes 1 to 8). Purification was performed using nickel-NTA Superflow utilizing the 6× His tag linked to an end of each protein.

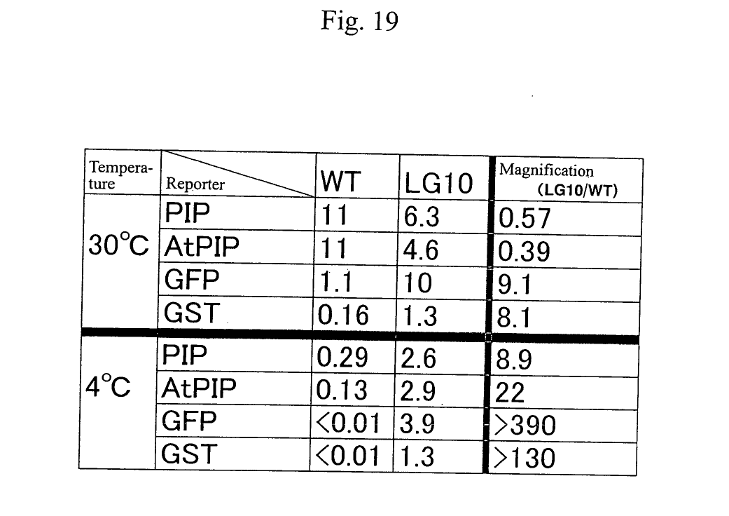

The results by measuring and quantifying the density of each band by densitometer are also shown in FIG. 19. These were quantified from the bands of SDS polyacrylamide electrophoresis shown in FIG. 18. In this figure, purified amount for each foreign protein from 1 liter of the culture solution is shown. The unit is shown by mg. The rightmost column (magnification) indicates that how many times of protein is purified when the protein is expressed using the TipA-LG10 promoter compared with the case where the protein is expressed using the TipA gene promoter. Consequently, it turns out that that the amount of the recombinant protein obtained at 4° C. was more when expressed from the TipA-LG10 promoter than from TipA gene promoter. However, in the case of 30° C., the amount of the recombinant protein obtained was not necessarily more when expressed from the TipA-LG10 promoter than from TipA gene promoter.

### EXAMPLE 19

Isolation of the Protein Derived from Mouse which Exhibits the Proliferation inhibiting Effect to E. coli at 30° C.

In order to investigate which gene exhibits the proliferation inhibiting effect to a host when expressed specifically, an expression library for E. coli was constructed using Poly(A)⁺RNA (product of STRATAGENE) derived from mouse liver. Details are specifically described below.

An arabinose inducible vector was adopted for use in the expression vector for E. coli. First, in order to facilitate introduction of cDNA into this vector, pBAD/HisA (product of Invitrogen), pBAD-Linker which has a modified multicloning site was created. The creation process is described below. Synthetic oligodeoxyribonucleotide described in SEQ ID Nos. 54 and 55 in the Sequence Listing which contain a cloning sequences consisting of EcoRI, BglII and XhoI recognition sites and have complementary sequence to each other are mixed in an equivalent molar amount, processes at 70° C. for 10 minutes, cooled down to room temperature for 20 minutes to form a double strand. As a result, the end parts thereof became linkable to a vector obtained by double digestion by NcoI and HindIII and was subcloned into NcoI and HindIII sites of pBAD/HisA. The resultant plasmid was named pBAD-Linker.

A double stranded cDNA was synthesized from the above-mentioned Poly(A)⁺RNA using cDNA synthesis kit available from STRATAGENE following the manufacturer's instructions. Subsequently, this cDNA was ligated into EcoRI and XhoI sites of pBAD-Linker. According to an ordinary method, this ligation product was transformed into E. coli TOP 10 (product of Invitrogen), and 50,000 transformants were acquired on LB agar culture medium containing 50 μg/ml ampicillin. A replica of this agar culture medium was created on LB agar culture medium which contained 50 μg/ml of ampicillin and 0.2% of L-arabinose by using Easy Transfer Replica Plating Device available from GenHunter, protein expression was induced, and incubated at 30° C. overnight. Consequently, 426 colonies which was able to grow on the culture medium not containing arabinose and was not able to grow on the culture medium containing arabinose were sorted out.

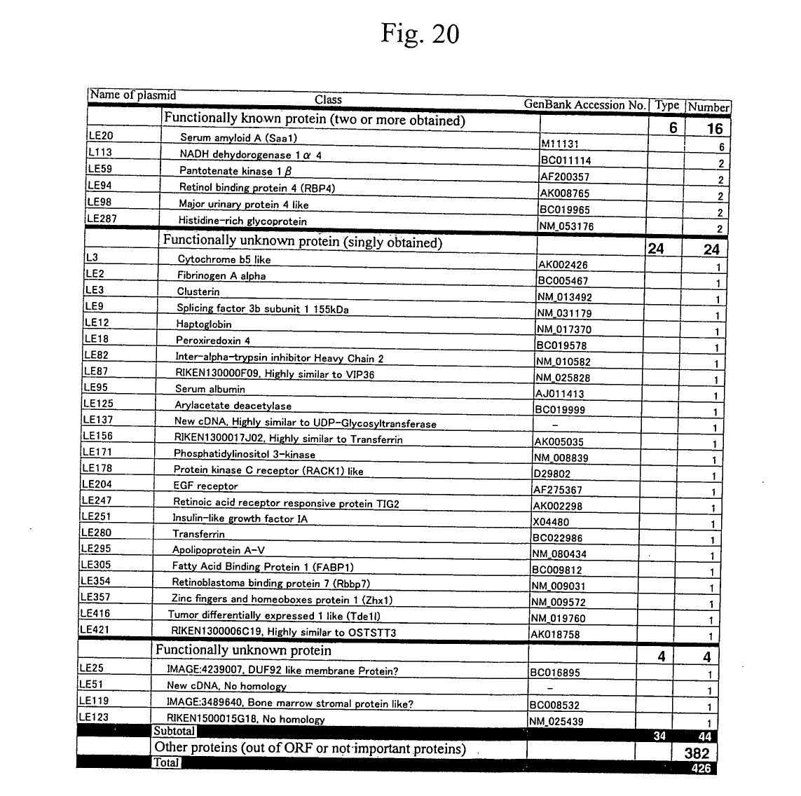

After culturing this 426 TOP10 transformant in 1.5 ml of LB culture medium containing 50 μg/ml ampicillin, plasmids were separated and purified following an ordinary method. After double digestion of restriction enzymes EcoRI and XhoI, the obtained plasmids were subjected to 1% agarose gel electrophoresis and the length of the cDNA fragment derived from mouse was estimated. Furthermore, from the obtained plasmid, the nucleotide sequence of the cDNA fragment derived from mouse was determined by about 500 bases using a synthetic oligodeoxyribonucleotides described in SEQ ID No. 56 in the Sequence Listing and a DNA sequencer ABI PRISM (R) 3100 Genetic Analyzer. The results are shown in FIG. 20. This figure shows the results using BLAST program, performing homology search based on the determined DNA sequence to identify the gene.

### EXAMPLE 20

Expression and Purification of Foreign Proteins in a Bacterium of Genus Rhodococcus and E. coli

Among the genes isolated in Example 19, Serum amyloid A (Saal), NADH dehydorogenase 1 alpha subcomplex 4, and Cytochrome b5 like, RIKEN1500015G18, Transferrin, Apolipoprotein A-V, Pantotenate kinase 1β, Peroxiredoxin 4 and RIKEN1300017J02 (Transferrin Homolog) were expressed using Rhodococcus erythropolis JCM3201 and E. coli TOP 10 as hosts. The following four groups and ten kinds of protein were expressed similarly.
1) Group 1: Three kinds of protease which is known to become an inactive inclusion body if expressed in E. coli, Cathepsin D, Prothrombin, Kallikrein 6;
2) Group 2: Two kinds of DNAses, LSDNAse and DLAD whose expression in E. coli is expected to be difficult from their physiology activity; and
3) Group 3: Those of which expression is considered to be difficult in E. coli according to the researches of other groups due to the cell proliferation inhibiting activity, HMG-1, Kid1, Bax alpha; and
4) Group 4: Those which are solubilized with low temperature dependence according to the researches of other groups, Glucokinase and p37A.

The recombinant proteins were expressed at 30° C. and 4° C. in Rhodococcus erythropolis and at 30° C. in E. coli, respectively. Details are described below.

Amplification by PCR was performed using plasmid LE20 as a template along with primers of SEQ ID Nos. 57 and 58 in the Sequence Listing. Consequently, DNA containing Saal gene (GenBank Accession Number M11131) encoding Serum Amyloid Protein A derived from mouse (Meeker et al., Proteins 30 381-387 (1998)) was obtained. Double digestion of this DNA fragment was carried out with restriction enzymes NdeI and XhoI, and the product was subcloned into NdeI and XhoI sites of pTip-LNH1. As a result, a plasmid containing Saal gene under control of the TipA-LG10 promoter was created, and the resultant plasmid was named pHN205. In addition, amplification by PCR was performed using plasmid LE20 as a template along with primers of SEQ ID Nos. 59 and 60 in the Sequence Listing. Consequently, DNA containing Saal gene derived from mouse was obtained. Double digestion of this DNA fragment was carried out with restriction enzymes XhoI and KpnI, and the product was subcloned into XhoI and KpnI sites of pBAD/HisA. As a result, a plasmid containing Saal gene under control of the arabinose inducible promoter was created, and the resultant plasmid was named pHN193.

Amplification by PCR was performed using plasmid L113 as a template along with primers of SEQ ID Nos. 61 and 62 in the Sequence Listing. Consequently, DNA containing a gene encoding NADH dehydrogenase 1 alpha subcomplex 4 derived from mouse (Walker et al., J. Mol. Biol. 226 1051-1072 (1992): GenBank Accession Number BC01114; hereinafter abbreviated as NADH4) was obtained. Double digestion of this DNA fragment was carried out with restriction enzymes NdeI and EcoRI, and the product was subcloned into NdeI and EcoRI sites of pTip-LNH1. As a result, a plasmid containing NADH4 gene under control of the TipA-LG10 promoter was created, and the resultant plasmid was named pHN206. In addition, amplification by PCR was performed using plasmid L113 as a template along with primers of SEQ ID Nos. 63 and 62 in the Sequence Listing. Consequently, DNA containing NADH4 gene derived from mouse was obtained. Double digestion of this DNA fragment was carried out with restriction enzymes XhoI and EcoRI, and the product was subcloned into XhoI and EcoRI sites of pBAD/HisA. As a result, a plasmid containing NADH4 gene under control of the arabinose inducible promoter was created, and the resultant plasmid was named pHN195.

Amplification by PCR was performed using plasmid L3 as a template along with primers of SEQ ID Nos. 64 and 65 in the Sequence Listing. Consequently, DNA containing a gene encoding Cytochrome b5 like protein derived from mouse (GenBank Accession Number AK002426: hereinafter abbreviated as Cytochrome b51) was obtained. Double digestion of this DNA fragment was carried out with restriction enzymes NdeI and EcoRI, and the product was subcloned into NdeI and EcoRI sites of pTip-LNH1. As a result, a plasmid containing Cytochrome b5l gene under control of the TipA-LG10 promoter was created, and the resultant plasmid was named pHN208. In addition, amplification by PCR was performed using plasmid L3 as a template along with primers of SEQ ID Nos. 66 and 65 in the Sequence Listing. Consequently, DNA containing Cytochrome b5l gene derived from mouse was obtained. Double digestion of this DNA fragment was carried out with restriction enzymes XhoI and EcoRI, and the product was subcloned into XhoI and EcoRI sites of pBAD/HisA. As a result, a plasmid containing Cytochrome b5l gene under control of the arabinose inducible promoter was created, and the resultant plasmid was named pHN199.

Amplification by PCR was performed using plasmid LE123 as a template along with primers of SEQ ID Nos. 67 and 68 in the Sequence Listing. Consequently, DNA containing a gene encoding a putative protein derived from mouse whose function was unknown (GenBank Accession Number NM#025439: hereinafter abbreviated as LE123) was obtained. Double digestion of this DNA fragment was carried out with restriction enzymes NdeI and EcoRI, and the product was subcloned into NdeI and EcoRI sites of pTip-LNH1. As a result, a plasmid containing LE123 gene under control of the TipA-LG10 promoter was created, and the resultant plasmid was named pHN287. In addition, amplification by PCR was performed using plasmid LE123 as a template along with primers of SEQ ID Nos. 69 and 68 in the Sequence Listing. Consequently, DNA containing LE123 gene derived from mouse was obtained. Double digestion of this DNA fragment was carried out with restriction enzymes XhoI and EcoRI, and the product was subcloned into XhoI and EcoRI sites of pBAD/HisA. As a result, a plasmid containing LE123 gene under control of the arabinose inducible promoter was created, and the resultant plasmid was named pHN276.

Amplification by PCR was performed using plasmid LE280 as a template along with primers of SEQ ID Nos. 70 and 71 in the Sequence Listing. Consequently, DNA containing a gene encoding Transferrin derived from mouse (Mason et al., Protein Expr. Purif. 23 142-150 (2001); GenBank Accession Number BC022986) was obtained. Double digestion of this DNA fragment was carried out with restriction enzymes NdeI and HindIII, and the product was subcloned into NdeI and HindIII sites of pTip-LNH1. As a result, a plasmid containing Transferrin gene under control of the TipA-LG10 promoter was created, and the resultant plasmid was named pHN289. In addition, amplification by PCR was performed using plasmid LE280 as a template along with primers of SEQ ID Nos. 72 and 71 in the Sequence Listing. Consequently, DNA containing Transferrin gene derived from mouse was obtained. Double digestion of this DNA fragment was carried out with restriction enzymes XhoI and HindIII, and the product was subcloned into XhoI and HindIII sites of pBAD/HisA. As a result, a plasmid containing Transferrin gene under control of the arabinose inducible promoter was created, and the resultant plasmid was named pHN277.

Amplification by PCR was performed using plasmid LE295 as a template along with primers of SEQ ID Nos. 73 and 74 in the Sequence Listing. Consequently, DNA containing a gene encoding Apolipoprotein A-V derived from mouse (van der Vliet et al., J. Biol. Chem. 276 44512-44520 (2001); GenBank Accession Number NM#080434; hereinafter referred to as Apoa5) was obtained. Double digestion of this DNA fragment was carried out with restriction enzymes NcoI and EcoRI, and the product was subcloned into NcoI and EcoRI sites of pTip-LNH2. As a result, a plasmid containing Apoa5 gene under control of the TipA-LG10 promoter was created, and the resultant plasmid was named pHN288. In addition, amplification by PCR was performed using plasmid LE295 as a template along with primers of SEQ ID Nos. 75 and 74 in the Sequence Listing. Consequently, DNA containing Apoa5 gene derived from mouse was obtained. Double digestion of this DNA fragment was carried out with restriction enzymes XhoI and EcoRI, and the product was subcloned into XhoI and EcoRI sites of pBAD/HisA. As a result, a plasmid containing Apoa5 gene under control of the arabinose inducible promoter was created, and the resultant plasmid was named pHN281.

Amplification by RT-PCR (Larrick, Trends Biotechnol. 10 146-152 (1992) was performed using mouse liver Poly(A)⁺RNA along with primers of SEQ ID Nos. 76 and 77 in the Sequence Listing. RT-PCR was performed by using ProSTAR Ultra HF RT-PCR System available from STRATAGENE following the manufacturer's instructions (hereinafter all RT-PCR was performed using this kit below). Consequently, DNA containing Cathepsin D gene derived from mouse (Grusby et al., Nucleic Acids Res. 18 4008 (1990); Babe et al., Biotechnology and Genetic Engineering Reviews 17 213-252:(2000); GenBank Accession Number X52886) was obtained. Double digestion of this DNA fragment was carried out with restriction enzymes NcoI and XhoI, and the product was subcloned into NcoI and XhoI sites of pTip-LCH1. As a result, a plasmid containing Cathepsin D gene under control of the TipA-LG10 promoter was created, and the resultant plasmid was named pHN270. Furthermore, a DNA fragment of 1.2 kb obtained by double digestion of pHN270 by NcoI and SalI was subcloned into NcoI and XhoI sites of pBAD/HisA. The resultant plasmid was named pHN273.

Amplification by RT-PCR was performed using mouse liver Poly(A)⁺RNA along with primers of SEQ ID Nos. 78 and 79 in the Sequence Listing. Consequently, DNA containing Prothrombin gene derived from mouse (Degen et al., DNA Cell Biol.9 487-498:(1990); GenBank Accession Number X52308) was obtained. Double digestion of this DNA fragment was carried out with restriction enzymes NcoI and XhoI, and the product was subcloned into NcoI and XhoI sites of pTip-LCH1. As a result, a plasmid containing Prothrombin gene under control of the TipA-LG10 promoter was created, and the resultant plasmid was named pHN271.

Amplification by RT-PCR was performed using mouse liver Poly(A)⁺RNA along with primers of SEQ ID Nos. 80 and 81 in the Sequence Listing. Consequently, DNA containing Kallikrein 6 gene derived from mouse (Evans et al., J.Biol.Chem.262 8027-8034 (1987); Babe et al., Biotechnology and Genetic Engineering Reviews 17 213-252:(2000); GenBank Accession Number NM#010639) was obtained. Double digestion of this DNA fragment was carried out with restriction enzymes NcoI and XhoI, and the product was subcloned into NcoI and XhoI sites of pTip-LC10. As a result, a plasmid containing Kallikrein 6 gene under control of the TipA-LG10 promoter was created, and the resultant plasmid was named pHN272. Furthermore, a DNA fragment of 0.7 kb obtained by double digestion of pHN272 by NcoI and SalI was subcloned into NcoI and XhoI sites of pBAD/HisA. The resultant plasmid was named pHN275.

Amplification by RT-PCR was performed using mouse liver Poly(A)⁺RNA along with primers of SEQ ID Nos. 82 and 83 in the Sequence Listing. Consequently, DNA containing LSDNAse gene derived from mouse (Baron et al., Gene 215 291-301:(1998); GenBank Accession Number AF 047355) was obtained. Double digestion of this DNA fragment was carried out with restriction enzymes NdeI and XhoI, and the product was subcloned into NdeI and XhoI sites of pTip-LNH1. As a result, a plasmid containing LSDNAse gene under control of the TipA-LG10 promoter was created, and the resultant plasmid was named pHN299.

Amplification by RT-PCR was performed using mouse liver Poly(A)⁺RNA along with primers of SEQ ID Nos. 84 and 85 in the Sequence Listing. Consequently, DNA containing DLAD gene derived from mouse (Shiokawa and Tanuma, Nucleic Acids Res.27 4083-4089:(1999); GenBank Accession Number AF 128888) was obtained. Double digestion of this DNA fragment was carried out with restriction enzymes NcoI and EcoRI, and the product was subcloned into NcoI and EcoRI sites of pTip-LNH2. As a result, a plasmid containing DLAD gene under control of the TipA-LG10 promoter was created, and the resultant plasmid was named pHN284.

Amplification by RT-PCR was performed using mouse liver Poly(A)+RNA along with primers of SEQ ID Nos. 86 and 87 in the Sequence Listing. Consequently, DNA containing HMG-1 gene derived from mouse (Pauken et al., Mamm.Genome 5 91-99 (1994); Lee et al., Gene 225 97-105:(1998); GenBank Accession Number U00431) was obtained. Double digestion of this DNA fragment was carried out with restriction enzymes NcoI and EcoRI, and the product was subcloned into NcoI and EcoRI sites of pTip-LNH2. As a result, a plasmid containing HMG-1 gene under control of the TipA-LG10 promoter was created, and the resultant plasmid was named pHN285. In addition, amplification by PCR was performed using plasmid pHN285 as a template along with primers of SEQ ID Nos. 88 and 87 in the Sequence Listing. Consequently, DNA containing HMG-1 gene derived from mouse was obtained. Double digestion of this DNA fragment was carried out with restriction enzymes NcoI and EcoRI, and the product was subcloned into NcoI and EcoRI sites of pBAD/HisA. As a result, a plasmid containing HMG-1 gene under control of the arabinose inducible promoter was created, and the resultant plasmid was named pHN305.

Amplification by RT-PCR was performed using mouse liver Poly(A)⁺RNA along with primers of SEQ ID Nos. 89 and 90 in the Sequence Listing. Consequently, DNA containing Kidl gene derived from mouse (Tekki-Kessaris et al., Gene 240 13-22 (1999); Suter-Crazzolara and Unsicker Bio/Technology 19 202-204:(1995); GenBank Accession Number AF 184111) was obtained. Double digestion of this DNA fragment was carried out with restriction enzymes NcoI and HindIII, and the product was subcloned into NcoI and HindIII sites of pTip-LNH2. As a result, a plasmid containing Kidl gene under control of the TipA-LG10 promoter was created, and the resultant plasmid was named pHN286.

Amplification by RT-PCR was performed using mouse liver Poly(A)⁺RNA along with primers of SEQ ID Nos. 91 and 92 in the Sequence Listing. Consequently, DNA containing Bax alpha gene derived from mouse (Oltvai et al., Cell 74 609-619 (1993); Donnelly et al., Protein Expr.Purif.22 422-429:(2001); GenBank Accession Number L22472) was obtained. Double digestion of this DNA fragment was carried out with restriction enzymes NdeI and EcoRI, and the product was subcloned into NdeI and EcoRI sites of pTip-LNH1. As a result, a plasmid containing Bax alpha gene under control of the TipA-LG10 promoter was created, and the resultant plasmid was named pHN217. In addition, amplification by PCR was performed using plasmid pHN217 as a template along with primers of SEQ ID Nos. 93 and 92 in the Sequence Listing. Consequently, DNA containing Bax alpha gene derived from mouse was obtained. Double digestion of this DNA fragment was carried out with restriction enzymes XhoI and EcoRI, and the product was subcloned into XhoI and EcoRI sites of pBAD/HisA. As a result, a plasmid containing Bax alpha gene under control of the arabinose inducible promoter was created, and the resultant plasmid was named pHN212.

Amplification by RT-PCR was performed using mouse liver Poly(A)⁺RNA along with primers of SEQ ID Nos. 94 and 95 in the Sequence Listing. Consequently, DNA containing Glucokinase gene derived from mouse (Lin et al., Protein Expr.Purif.1 169-176(1990); GenBank Accession Number BC011139) was obtained. Double digestion of this DNA fragment was carried out with restriction enzymes NdeI and XhoI, and the product was subcloned into NdeI and XhoI sites of pTip-LNH1. As a result, a plasmid containing Glucokinase gene under control of the TipA-LG10 promoter was created, and the resultant plasmid was named pHN298. Furthermore, a DNA fragment of 1.4 kb obtained by double digestion of pHN298 by NcoI and XhoI was subcloned into NcoI and XhoI sites of pBAD/HisA. The resultant plasmid was named pHN306.

Amplification by PCR was performed using plasmid pET22 b-Dmp37A as a template along with primers of SEQ ID Nos. 105 and 96 in the Sequence Listing. Consequently, DNA containing a gene encoding p37A (Holzl et al., J.Cell Biol.150 119-129:(2000); GenBank Accession Number AF145312) derived from Drosophila melanogaster was obtained. Double digestion of this DNA fragment was carried out with restriction enzymes NdeI and XhoI, and the product was subcloned into NdeI and XhoI sites of pTip-LCH2. As a result, a plasmid containing p37A gene under control of the TipA-LG10 promoter was created, and the resultant plasmid was named pHN291. In addition, amplification by PCR was performed using plasmid pHN291 as a template along with primers of SEQ ID Nos. 97 and 25 in the Sequence Listing. Consequently, DNA containing p37A gene derived from Drosophila melanogaster was obtained. This DNA fragment was digested by restriction enzymes SalI and then partially digested by NcoI (so that digestion might not occur at NcoI inside p37A), and the product was subcloned into NcoI and XhoI sites of pBAD/HisA. As a result, a plasmid containing p3 7A gene under control of the arabinose inducible promoter was created, and the resultant plasmid was named pHN308.

Amplification by PCR was performed using plasmid LE59 as a template along with primers of SEQ ID Nos. 98 and 99 in the Sequence Listing. Consequently, DNA containing a gene encoding Pantothenate kinase I beta protein (Rock et al., J. Biol.Chem.275 1377-1383:(2000); GenBank Accession Number AF200357—hereinafter abbreviated as PanK) derived from mouse was obtained. Double digestion of this DNA fragment was carried out with restriction enzymes XhoI and EcoRI, and the product was subcloned into XhoI and EcoRI sites of pBAD/HisA. As a result, a plasmid containing PanK gene under control of the arabinose inducible promoter was created, and the resultant plasmid was named pHN279.

Amplification by RT-PCR was performed using mouse liver Poly(A)⁺RNA along with primers of SEQ ID Nos. 100 and 101 in the Sequence Listing. Consequently, DNA containing a gene encoding Peroxiredoxin 4 derived from mouse (GenBank Accession Number BC019578) was obtained. Double digestion of this DNA fragment was carried out with restriction enzymes XhoI and KpnI, and the product was subcloned into XhoI and KpnI sites of pBAD/HisA. As a result, a plasmid containing Peroxiredoxin 4 gene under control of the arabinose inducible promoter was created, and the resultant plasmid was named pHN278.

Amplification by PCR was performed using plasmid LE156 as a template along with primers of SEQ ID Nos. 102 and 103 in the Sequence Listing. Consequently, DNA containing a gene encoding Transferrin-like protein (GenBank Accession Number AK005035; hereinafter abbreviated as TFL) derived from mouse was obtained. Double digestion of this DNA fragment was carried out with restriction enzymes XhoI and EcoRI, and the product was subcloned into XhoI and EcoRI sites of pBAD/HisA. As a result, a plasmid containing TFL gene under control of the arabinose inducible promoter was created, and the resultant plasmid was named pHN280.

All the proteins mentioned above that have signal peptide were subcloned without the DNA sequence encoding signal peptide. As for Prothrombin, the DNA sequence encoding “Prethrombin-2” immediately before Prothrombin becomes a matured Thrombin (Soejima et al., J.Biochem.130 269-277 (2001)) was subcloned.

The transformation of Rhodococcus erythropolis JCM3201 strain was carried out in the same manner as in Example 12 using pHN1 71 (described in Example 7), pHN205, pHN206, pHN208, pHN287, pHN289, pHN288, pHN270, pHN271, pHN272, pHN299, pHN284, pHN285, pHN286, pHN217, pHN298 and pHN291, respectively to express each protein at 30° C. and 4° C. in the similar manner as in Example 13.

All of these proteins had 6× His tag linked to the end and were purified as in Example 16. In addition to this, they were purified at this time also from the precipitation (described in Example 16) formed by centrifugation at 20,000×g after cell destruction. The purification process from the precipitation was shown below specifically, and the procedure was conducted at room temperature. The precipitation was suspended into 1 ml DN-Buffer (50 mM Tris-HCl (pH 8.0), 8 M urea) and centrifuged at 20,000×g, and nickel-NTA Superflow which was beforehand equilibrated with DT-Buffer was added to 700 μl of the supernatant so that the bed volume might be 40 μl. This mixture was subjected to rotation agitation for 1 hour to combine the nickel-NTA Superflow beads to the protein attached with 6×His tag. After washing these beads 4 times by NT-Buffer, the protein was eluted from the protein attached with 6×His tag by suspending these beads in 120 μl of DNE-Buffer (50 mM Tris-HCl (pH 7.0), 8 M urea, 400 mM imidazole) 3 times.

Proteins were expressed in E. coli using pBAD/His/lacZ (Invitrogen), pHN193, pHN195, pHN199, pHN276, pHN277, pHN281, pHN273, pHN275, pHN305, pHN212, pHN306, pHN308, pHN279, pHN278 and pHN280 following the manufacturer's instructions of the pBAD/His kit of Invitrogen.

The purification process from the precipitation was shown below specifically. The cell bodies which expressed a protein were collected and suspended into I ml of NT-Buffer. The cells were destroyed by using an ultrasonic generator UD-20 (product of TOMY). The mixture was centrifuged at 20,000×g, and nickel-NTA Superflow which was beforehand equilibrated with NT-Buffer was added to 900 μl of the supernatant so that the bed volume might be 40 μl. This mixture was subjected to rotation agitation for 1 hour to combine the nickel-NTA Superflow beads to the protein attached with 6× His tag. After washing these beads 4 times by NT-Buffer, the protein was eluted from the protein attached with 6× His tag by suspending these beads in 120 μl of NTE-Buffer 3 times. All of the above-mentioned procedure were conducted at 4° C.

Purification was performed also from the precipitation formed by centrifugation at 20,000×g after cell destruction, the procedure therefore was the same as that of the method mentioned above.

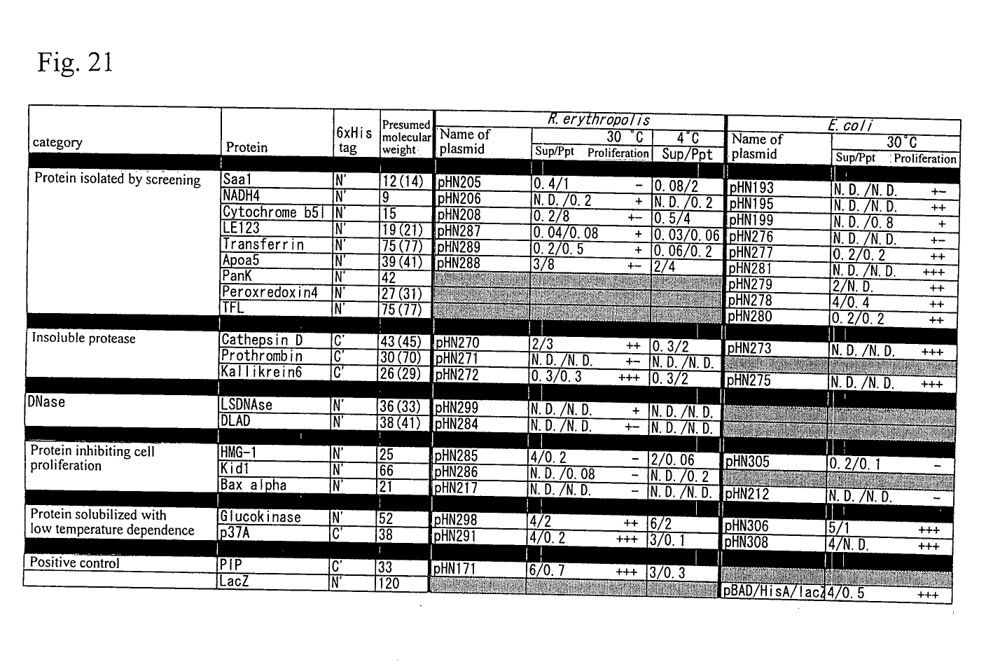

10 μl of the above-mentioned samples was subjected to 12% SDS polyacrylamide electrophoresis following an ordinary method, and the density of the bands were measured and quantified by densitometer and the results are shown in FIG. 21. In this figure, the second column from the left shows the name of the protein expressed. The third column from the left shows to which of the N-terminal or C-terminal the 6×His tag was attached. The fourth column from the left shows presumed molecular weight (kDa) of the full-length protein including signal sequence etc., and the number in parentheses shows presumed molecular weight of the actually expressed portion of the protein. The fifth and ninth columns from the left show the name of the plasmid used when protein was expressed. The sixth, eighth and tenth columns from the left show the mass of the recombinant protein obtained (unit: milligram) per liter and the results purified from the supernatant fraction (Sup) centrifuged at 20,000×g and those purified from the precipitation fraction (Ppt) are separately shown. The symbols + and − in the seventh and eleventh columns from the left show the proliferation rate when each transformant was applied on the agar culture medium containing an expression induction agent (1 μg/ml of thiostrepton in the case of Rhodococcus erythropolis and 0.2% L-arabinose in the case of E. coli). The transformant proliferated most rapidly is indicated by “+++”, and the transformant which did not proliferate at all is indicated by “−”. The host used, and the temperature at the time of expression induction are also shown in the topmost part. N.D. (Not Detected) shows that the result was below the detection limit.

The list of each plasmid used in Examples is shown in Table 1 and list of the strains used in Examples is shown in Table 2.

TABLE 1

| Category | Name of Plasmid | Notes | Source |
|---|---|---|---|
| Comercially | pBluescript | Conventional vector for | Stratagene |
| available | II SK (+) | general cloning | |
| cloning | pGEM 3Z | Conventional vector for | Promega |
| vector | (f)+ | general cloning | |
| Sources of | pRE2895 | Source of RepA&B | This study |
| expression | | (Cryptic plasmid isolated | |
| vector | | from R. erythropolis | |
| | | JCM2895) | |
| | pHN136 | Backborne of the | This study |
| | | expression vector | |
| | pHN143 | Source of Thio^{r} | This study |
| | pHN62 | Source of ALDHp-TipA | This study |
| | | (Inducer cassette) | |
| | pHN153 | Source of TipAp-PIP | This study |
| | | ORF-ALDHt (Expression | |
| | | cassette) | |
| | pHN169 | Source of Tuf1p-Tet^{r} | This study |
| Control | pHN172 | Neither Expression | This study |
| plasmid | | cassette nor Inducer | |
| | | cassette | |
| | pHN173 | Expression cassette, but | This study |
| | | no Inducer cassette | |
| Expression | pTip-NH1 | TipAp, 6 × His at | This study |
| plasmid | | N-terminus, MCS type1 | |
| in R. | pTip-CH1 | TipAp, 6 × His at | This study |
| erythropolis | | C-terminus, MCS type1 | |
| (not | pTip-NH2 | TipAp, 6 × His at | This study |
| containing | | N-terminus, MCS type2 | |
| target gene) | | | |
| | pTip-CH2 | TipAp, 6 × His at | This study |
| | | C-terminus, MCS type2 | |
| | pTip-LNH1 | TipA-LG10p, 6 × His at | This study |
| | | N-terminus, MCS type1 | |
| | pTip-LCH1 | TipA-LG10p, 6 × His at | This study |
| | | C-terminus, MCS type1 | |
| | pTip-LNH2 | TipA-LG10p, 6 × His at | This study |
| | | N-terminus, MCS type2 | |
| | pTip-LCH2 | TipA-LG10p, 6 × His at | This study |
| | | C-terminus, MCS type2 | |
| | pTip-CH1.1 | TipAp, 6 × His at | This study |
| | | N-terminus, MCS type3 | |
| | pTip-CH2.1 | TipAp, 6 × His at | This study |
| | | C-terminus, MCS type3 | |
| | pTip-LCH1.1 | TipA-LG10p, 6 × His at | This study |
| | | N-terminus, MCS type3 | |
| | pTip-LCH2.1 | TipA-LG10p, 6 × His at | This study |
| | | C-terminus, MCS type3 | |
| Expression | pHN170 | Target = PIP in | This study |
| plasmid | | pTip-CH1 | |
| in R. | pHN171 | Target = PIP in | This study |
| erythropolis | | pTip-LCH1 | |
| (containing | pHN176 | Targets = AtPIP in | This study |
| target gene) | | pTip-CH1 | |
| | pHN177 | Targets = AtPIP in | This study |
| | | pTip-LCH1 | |
| | pHN187 | Target = GFP in | This study |
| | | pTip-NH1 | |
| | pHN186 | Target = GFP in | This study |
| | | pTip-LNH1 | |
| | pHN282 | Target = GST in | This study |
| | | pTip-NH2 | |
| | pHN283 | Target = GST in | This study |
| | | pTip-LNH2 | |
| | pHN205 | Target = Saa1 in | This study |
| | | pTip-LNH1 | |
| | pHN206 | Target = NADH4 in | This study |
| | | pTip-LNH1 | |
| | pHN208 | Target = Cytochrome | This study |
| | | b5l in pTip-LNH1 | |
| | pHN287 | Target = LE123 in | This study |
| | | pTip-LNH1 | |
| | pHN289 | Target = Transferrin | This study |
| | | in pTip-LNH1 | |
| | pHN288 | Target = Apoa5 in | This study |
| | | pTip-LNH2 | |
| | pHN270 | Target = Cathepsin | This study |
| | | D in pTip-LCH1 | |
| | pHN271 | Target = Prothrombin | This study |
| | | in pTip-LCH1 | |
| | pHN272 | Target = Kallikrein6 | This study |
| | | in pTip-LCH1 | |
| | pHN299 | Target = LSDNase in | This study |
| | | pTip-LNH1 | |
| | pHN284 | Target = DLAD in | This study |
| | | pTip-LNH2 | |
| | pHN285 | Target = HMG-1 in | This study |
| | | pTip-LNH2 | |
| | pHN286 | Target = Kid1 in | This study |
| | | pTip-LNH2 | |
| | pHN217 | Target = Bax alpha | This study |
| | | in pTip-LNH1 | |
| | pHN298 | Target = Glucokinase | This study |
| | | in pTip-LNH1 | |
| | pHN291 | Target = p37A in | This study |
| | | pTip-LCH2 | |
| Expression | pBAD/HisA | BAD promoter, 6 × His at | Invitrogen |
| plasmid in | | N-terminus, Xpress | |
| E. coli (not | | Epitope, MCS | |
| containing | pBAD-Linker | BAD promoter, for | This study |
| target gene) | | library construction | |
| Expression | pHN193 | Target = Saa1 in | This study |
| plasmid in | | pBAD/HisA | |
| E. coli | pHN195 | Target = NADH4 in | This study |
| (containing | | pBAD/HisA | |
| target gene) | pHN199 | Target = Cytochrome | This study |
| | | b5l in pBAD/HisA | |
| | pHN276 | Target = LE123 in | This study |
| | | pBAD/HisA | |
| | pHN277 | Target = Transferrin | This study |
| | | in pBAD/HisA | |
| | pHN281 | Target = Apoa5 in | This study |
| | | pBAD/HisA | |
| | pHN273 | Target = Cathepsin | This study |
| | | D in pBAD/HisA | |
| | pHN275 | Target = Kallikrein6 | This study |
| | | in pBAD/HisA | |
| | pHN305 | Target = HMG-1 in | This study |
| | | pBAD/HisA | |
| | pHN212 | Target = Bax alpha | This study |
| | | in pBAD/HisA | |
| | pHN306 | Target = Glucokinase | This study |
| | | in pBAD/HisA | |
| | pHN308 | Target = p37A in | This study |
| | | pBAD/HisA | |
| | pHN279 | Target = PanK in | This study |
| | | pBAD/HisA | |
| | pHN278 | Target = | This study |
| | | Peroxiredoxin4 in | |
| | | pBAD/HisA | |
| | pHN280 | Target = TFL in | This study |
| | | pBAD/HisA | |
| | pBAD/ | Target = lacZ | Invitrogen |
| | His/lacZ | | |

TABLE 2

| | Name of | | | |
|---|---|---|---|---|
| Speices | strain | Alias name | Source | Use |
| Rhodococcus | JCM2895 | ATCC15962 | Japan Collection of Microorganisms | Source of pRE2895 |
| erythropolis | | | | (Source of RepA&B) |
| Rhodococcus | JCM3201 | ATCC4277 | Japan Collection of Microorganisms | Host strain to express |
| erythropolis | | | | recombinant proteins |
| | | | | Source of ALDHp |
| | | | | Source of ALDHt |
| Rhodococcus | JCM10002 | ATCC12974 | Japan Collection of Microorganisms | Host strain to express |
| fascians | | | | recombinant proteins |
| Rhodococcus | DSM44193 | PD630 | German Collection of Microorganisms | Host strain to express |
| opacus | | | and Cell Cultures | recombinant proteins |
| Streptomyces | JCM4979 | A3 (2) | Japan Collection | Source of TipA |
| coelicolor | | | of Microorganisms | Source of Tuf1p |
| Streptomyces | JCM4217 | ATCC14921 | Japan Collection of Microorganisms | Source of Thio^{r} |
| azureus | | | | |
| Escherichia coli | T0P10 | | Invitrogen | Host strain to express |
| | | | | recombinant proteins |
| Escherichia coli | DH5α | | | General cloning |

All the publications, patent, and patent application cited in this specification are entirely incorporated as references.

### INDUSTRIAL APPLICABILITY

As shown in Examples 13 to 18 and Example 20, expression and production of a protein encoded by a foreign gene are enabled under low temperature conditions of 4° C. by using the expression vector of the present invention.
Free words in the Sequence Listing
Sequences 1 to 105: primers
Sequences 106 to 113: vectors

1. An expression vector which can inducibly express a foreign gene with an inducing substance in a host cell and can be expressed at a temperature equal to or below the suitable growth temperature range of a host other than said host.

2. An expression vector which can inducibly express a foreign gene with an inducing substance in a host cell and can be expressed at a temperature of 15° C. or lower.

3. The expression vector according to claim 1 or 2 which can be expressed at 4° C.

4. The expression vector according to any one of claims 1 to 3 wherein the host cell is a bacterium of genus Rhodococcus.

5. The expression vector according to claim 4 wherein the bacterium of genus Rhodococcus is selected from the group consisting of R. erythropolis, R. fascians and R. opacus.

6. The expression vector according to any one of claims 1 to 5 wherein the inducing substance is thiostrepton.

7. The expression vector according to any one of claims 1 to 6 wherein the foreign gene encodes a protein which inhibits proliferation of the host cell under medium to high temperature conditions exceeding 15° C.

8. The expression vector according to any one of claims 1 to 7 wherein the vector comprises a promoter sequence whose expression can be regulated by an inducing substance and a multicloning site to which a foreign gene can be introduced.

9. A transformant comprising an expression vector according to any one of claims 1 to 8.

10. A method for producing protein using an expression vector according to any one of claims 1 to 8.

11. An inducible expression vector which can inducibly express a gene encoding a protein which inhibits proliferation of the host cell when it is expressed at a temperature within the range of suitable growth temperature of the host cell in another host cell having a suitable growth temperature range lower than the suitable growth temperature of said host cell using an inducing substance.

12. The expression vector according to claim 11 which can be expressed at 4° C.

13. The expression vector according to claim 11 or 12 wherein the host cell is a bacterium of genus Rhodococcus.

14. The expression vector according to claim 13 wherein the bacterium of genus Rhodococcus is selected from the group consisting of R. erythropolis, R. fascians and R. opacus.

15. The expression vector according to any one of claims 11 to 14 wherein the inducing substance is thiostrepton.

16. The expression vector according to any one of claims 11 to 15 wherein the vector comprises a promoter sequence whose expression can be regulated by an inducing substance and a multicloning site to which a foreign gene can be introduced.

17. A transformant comprising an expression vector according to any one of claims 11 to 16.

18. A method for producing a protein using an expression vector according to any one of claims 11 to 16.

19. An expression vector which can inducibly express a foreign gene using an inducing substance in a bacterium of genus Rhodococcus.

20. The expression vector according to claim 19 wherein the bacterium of genus Rhodococcus is selected from the group consisting of R. erythropolis, R. fascians and R. opacus.

21. The expression vector according to claim 19 or 20 wherein the inducing substance is thiostrepton.

22. An expression vector according to any one of claims 19 to 21 wherein the vector comprises an expression cassette comprising a TipA gene promoter sequence, the first multicloning site to which a foreign gene can be introduced and a transcription termination sequence, an inducer cassette comprising the second promoter sequence and TipA gene, a DNA region essential to autonomous replication of a plasmid for a bacterium of genus Rhodococcus and a thiostrepton resistance gene.

23. A transformant of a bacterium of genus Rhodococcus comprising an expression vector according to any one of claims 19 to 22.

24. A method for producing a protein using an expression vector according to any one of claims 19 to 22.

25. An inducible expression vector for a bacterium of genus Rhodococcus which can inducibly express a gene encoding a protein whose expression product inhibits proliferation of the host cell under medium to high temperature conditions exceeding 15° C. in a bacterium of genus Rhodococcus which can proliferate under low-temperature conditions.

26. An inducible expression vector for a bacterium of genus Rhodococcus whose expression can be induced in a bacterium of genus Rhodococcus in which a foreign gene can be multiplied under low-temperature conditions, wherein the vector comprises an expression cassette comprising a TipA gene promoter sequence, the first multicloning site to which a foreign gene can be introduced and a transcription termination sequence, an inducer cassette comprising the second promoter sequence and TipA gene, a DNA region essential to autonomous replication of a plasmid for a bacterium of genus Rhodococcus and a thiostrepton resistance gene.

27. The inducible expression vector for a bacterium of genus Rhodococcus according to claim 26 wherein the vector further comprises a DNA region essential to autonomous replication of a plasmid for E. coli and can be reproduced in E. coli.

28. The inducible expression vector for a bacterium of genus Rhodococcus according to claim 26 or 27 wherein the TipA gene promoter is TipA-LG10 promoter.

29. The inducible expression vector for a bacterium of genus Rhodococcus according to any one of claims 26 to 28 wherein the vector is selected from the group consisting of pTip-NH1 having a nucleotide sequence represented by SEQ ID No. 106, pTip-NH2 having a nucleotide sequence represented by SEQ ID No. 107, pTip-CH1 having a nucleotide sequence represented by SEQ ID No. 108, pTip-CH2 having a nucleotide sequence represented by SEQ ID No. 109, pTip-LNH1 having a nucleotide sequence represented by SEQ ID No. 110, pTip-LNH2 having a nucleotide sequence represented by SEQ ID No. 111, pTip-LCH1 having a nucleotide sequence represented by SEQ ID No. 112, pTip-LCH2 having a nucleotide sequence represented by SEQ ID No. 113, pTip-CH1.1, pTip-CH2.1, pTip-LCH1.1 and pTip-LCH2.1.

30. The inducible expression vector for a bacterium of genus Rhodococcus according to any one of claims 25 to 29 wherein the bacterium of genus Rhodococcus is selected from the group consisting of R. erythropolis, R. fascians and R. opacus.

31. A transformant of a bacterium of genus Rhodococcus comprising an inducible expression vector for a bacterium of genus Rhodococcus according to any one of claims 25 to 30.

32. A process for producing a protein whose expression product inhibits proliferation of the host cell under medium to high temperature conditions exceeding 15° C. wherein the process comprises introducing an inducible expression vector for a bacterium of genus Rhodococcus according to any one of claims 25 to 30 comprising as a foreign gene a gene encoding a protein difficult to be expressed under medium to high temperature conditions exceeding 15° C. into a bacterium of genus Rhodococcus which can proliferate at low temperature; and culturing the above-mentioned bacteria into which an inducible expression vector for a bacterium of genus Rhodococcus is introduced using a culture medium comprising thiostrepton under low-temperature.

33. The process for producing a protein at low temperature according to claim 32 wherein the protein difficult to be expressed under medium to high temperature conditions exceeding 15° C. is a protein which inhibits proliferation of the host cell under medium to high temperature conditions exceeding 15° C.

34. The process for producing a protein at low temperature according to claim 32 wherein the protein difficult to be expressed under medium to high temperature conditions exceeding 15° C. is a protein which forms an inactive inclusion body in E. coli when it is made expressed under medium to high temperature conditions exceeding 15° C.

35. A process for producing a protein derived from psychrotrophic bacteria, heterothermic animals or plants living under a low-temperature environment wherein the process comprises introducing an inducible expression vector for a bacterium of genus Rhodococcus according to any one of claims 25 to 30 comprising a gene encoding a protein derived from psychrotrophic bacteria, heterothermic animals or plants living under a low-temperature environment into a bacterium of genus Rhodococcus which can proliferate at low temperature; and culturing the above-mentioned bacteria into which an inducible expression vector for a bacterium of genus Rhodococcus is introduced using a culture medium comprising thiostrepton under low-temperature.

36. A process for screening a protein difficult to be expressed under medium to high temperature conditions exceeding 15° C. wherein the process comprises introducing an inducible expression vector for a bacterium of genus Rhodococcus according to any one of claims 25 to 30 comprising a foreign gene into a bacterium of genus Rhodococcus which can proliferate at low temperature; culturing the above-mentioned bacteria into which an inducible expression vector for a bacterium of genus Rhodococcus is introduced using a culture medium comprising thiostrepton under medium to high temperature conditions exceeding 15° C. and under low-temperature condition; and selecting a gene which can be expressed only under low-temperature condition equal to or below 15° C.

37. The process for screening a protein difficult to be expressed under medium to high temperature conditions exceeding 15° C. according to claim 36 wherein the protein difficult to be expressed under medium to high temperature conditions exceeding 15° C. is a protein which inhibits proliferation of the host cell under medium to high temperature conditions exceeding 15° C.

38. A process for screening a protein difficult to be expressed under medium to high temperature conditions exceeding 15° C. wherein the process comprises selecting a gene which does not express or inhibits proliferation of E. coli when it is introduced and made expressed under medium to high temperature conditions exceeding 15° C.; and then introducing an inducible expression vector for a bacterium of genus Rhodococcus according to any one of claims 25 to 30 comprising said gene as a foreign gene into a bacterium of genus Rhodococcus which can proliferate at low temperature; and selecting a gene which can be expressed when cultured under low-temperature condition using a culture medium comprising thiostrepton.

39. The process for screening a protein according to claim 38 wherein the protein difficult to be expressed under medium to high temperature conditions exceeding 15° C. is a protein which inhibits proliferation of E. coli at 30° C. or higher.

40. The process for screening a protein according to claim 38 wherein the protein difficult to be expressed under medium to high temperature conditions exceeding 15° C. is a protein which forms an inclusion body when it is made expressed under medium to high temperature conditions exceeding 15° C.

41. The process for screening a protein according to claim 38 wherein the protein difficult to be expressed under medium to high temperature conditions exceeding 15° C. is a protein which inhibits proliferation of the host cell under medium to high temperature conditions exceeding 15° C.

42. A protein difficult to be expressed under medium to high temperature conditions exceeding 15° C. obtained by a screening process according to any one of claims 36 to 41.


SEQUENCE LISTING (131 sequences)

SEQ ID NO: 1 - DNA, 25 bp, Artificial Sequence
  Description of Artificial Sequence Synthetic primer sHN1
cagagctcgtcaggtggcacttttc

SEQ ID NO: 2 - DNA, 30 bp, Artificial Sequence
  Description of Artificial Sequence Synthetic primer sHN2
gttgtacaactagtcgtgccagctgcatta

SEQ ID NO: 3 - DNA, 26 bp, Artificial Sequence
  Description of Artificial Sequence Synthetic primer sHN120
gctgtacacccgagaagctcccagcg

SEQ ID NO: 4 - DNA, 29 bp, Artificial Sequence
  Description of Artificial Sequence Synthetic primer sHN121
cggagctcttgaacgagagttggccgttg

SEQ ID NO: 5 - DNA, 39 bp, Artificial Sequence
  Description of Artificial Sequence Synthetic primer sHN122
tcagatctatcgtcatcgactgcgatcacgttgacgccg

SEQ ID NO: 6 - DNA, 31 bp, Artificial Sequence
  Description of Artificial Sequence Synthetic primer sHN123
acggatcctccgctgaaatctcgccgtgcct

SEQ ID NO: 7 - DNA, 28 bp, Artificial Sequence
  Description of Artificial Sequence Synthetic primer sHN130
cttcatatgcggagctcgaccgcgcggg

SEQ ID NO: 8 - DNA, 24 bp, Artificial Sequence
  Description of Artificial Sequence Synthetic primer sHN131
atcgagtcgttcaagggcgtcggc

SEQ ID NO: 9 - DNA, 23 bp, Artificial Sequence
  Description of Artificial Sequence Synthetic primer NEB1233
agcggataacaatttcacacagg

SEQ ID NO: 10 - DNA, 19 bp, Artificial Sequence
  Description of Artificial Sequence Synthetic primer sHN10
caccaggatgatccccgac

SEQ ID NO: 11 - DNA, 18 bp, Artificial Sequence
  Description of Artificial Sequence Synthetic primer sHN11
gacagtgacatcaccagc

SEQ ID NO: 12 - DNA, 24 bp, Artificial Sequence
  Description of Artificial Sequence Synthetic primer NEB1224
cgccagggttttcccagtcacgac

SEQ ID NO: 13 - DNA, 24 bp, Artificial Sequence
  Description of Artificial Sequence Synthetic primer sHN40
atgagctactccgtgggacaggtg

SEQ ID NO: 14 - DNA, 29 bp, Artificial Sequence
  Description of Artificial Sequence Synthetic primer sHN41
tgcagatcttccgtttcgacgtgacggag

SEQ ID NO: 15 - DNA, 26 bp, Artificial Sequence
  Description of Artificial Sequence Synthetic primer sHN42
cagtctagaattgatctcctcgaccg

SEQ ID NO: 16 - DNA, 20 bp, Artificial Sequence
  Description of Artificial Sequence Synthetic primer sHN43
tgcaagctcctatgtaaacg

SEQ ID NO: 17 - DNA, 19 bp, Artificial Sequence
  Description of Artificial Sequence Synthetic primer sHN55
cgcctgctccacggccgcc

SEQ ID NO: 18 - DNA, 18 bp, Artificial Sequence
  Description of Artificial Sequence Synthetic primer sHN56
atggaggcacgcagcatg

SEQ ID NO: 19 - DNA, 19 bp, Artificial Sequence
  Description of Artificial Sequence Synthetic primer sHN57
cgccccctcggagtcggcg

SEQ ID NO: 20 - DNA, 18 bp, Artificial Sequence
  Description of Artificial Sequence Synthetic primer sHN58
atggacgccgccgaggac

SEQ ID NO: 21 - DNA, 26 bp, Artificial Sequence
  Description of Artificial Sequence Synthetic primer sHN147
cgtgtacatatcgaggcgggctccca

SEQ ID NO: 22 - DNA, 31 bp, Artificial Sequence
  Description of Artificial Sequence Synthetic primer sHN39
atccatggccgctcccttctctgacgccgtc

SEQ ID NO: 23 - DNA, 22 bp, Artificial Sequence
  Description of Artificial Sequence Synthetic primer sHN36
accatggatcaggaatgcatag

SEQ ID NO: 24 - DNA, 59 bp, Artificial Sequence
  Description of Artificial Sequence Synthetic primer sHN37
ttactagtttattaatgatgatgatgatgatgcaggtgtttcaggatgaaatccgaaag

SEQ ID NO: 25 - DNA, 29 bp, Artificial Sequence
  Description of Artificial Sequence Synthetic primer sHN6
cgtctagagtcccgctgaggcggcgtagc

SEQ ID NO: 26 - DNA, 29 bp, Artificial Sequence
  Description of Artificial Sequence Synthetic primer sHN9
ctactagtcgacccaccggcacccgtgag

SEQ ID NO: 27 - DNA, 30 bp, Artificial Sequence
  Description of Artificial Sequence Synthetic primer sHN141
aatctagagtaacgggctactccgtttaac

SEQ ID NO: 28 - DNA, 30 bp, Artificial Sequence
  Description of Artificial Sequence Synthetic primer sHN142
gggtcgacggtcctcctgtggagtggttct

SEQ ID NO: 29 - DNA, 33 bp, Artificial Sequence
  Description of Artificial Sequence Synthetic primer sHN145
gcactcgagatgaaatctaacaatgcgctcatc

SEQ ID NO: 30 - DNA, 30 bp, Artificial Sequence
  Description of Artificial Sequence Synthetic primer sHN152
agactagtcctcaacgacaggagcacgatc

SEQ ID NO: 31 - DNA, 22 bp, Artificial Sequence
  Description of Artificial Sequence Synthetic primer T7
gtaatacgactcactatagggc

SEQ ID NO: 32 - DNA, 20 bp, Artificial Sequence
  Description of Artificial Sequence Synthetic primer sHN153
aatccacaggacgggtgtgg

SEQ ID NO: 33 - DNA, 19 bp, Artificial Sequence
  Description of Artificial Sequence Synthetic primer sHN154
ctctacgccggacgcatcg

SEQ ID NO: 34 - DNA, 22 bp, Artificial Sequence
  Description of Artificial Sequence Synthetic primer T3
gcaattaaccctcactaaaggg

SEQ ID NO: 35 - DNA, 20 bp, Artificial Sequence
  Description of Artificial Sequence Synthetic primer sHN155
acgacgctctcccttatgcg

SEQ ID NO: 36 - DNA, 19 bp, Artificial Sequence
  Description of Artificial Sequence Synthetic primer sHN156
ccgatgcccttgagagcct

SEQ ID NO: 37 - DNA, 67 bp, Artificial Sequence
  Description of Artificial Sequence Synthetic primer sHN110
aaccatggtatatctccttcttaaagttaaacaaaattatttctagacgccgtccacgctgcctcct

SEQ ID NO: 38 - DNA, 77 bp, Artificial Sequence
  Description of Artificial Sequence Synthetic primer NNco1
catgggccaccatcaccatcaccatatgggaattctacgtagcggccgcggatccaagcttagatctcgaggatgaa

SEQ ID NO: 39 - DNA, 77 bp, Artificial Sequence
  Description of Artificial Sequence Synthetic primer NNco2
ctagttcatcctcgagatctaagcttggatccgcggccgctacgtagaattcccatatggtgatggtgatggtggcc

SEQ ID NO: 40 - DNA, 71 bp, Artificial Sequence
  Description of Artificial Sequence Synthetic primer CNco1
catgggaattctacgtagcggccgcggatccaagcttagatctcgaggacatcaccatcaccatcactgaa

SEQ ID NO: 41 - DNA, 71 bp, Artificial Sequence
  Description of Artificial Sequence Synthetic primer CNco2
ctagttcagtgatggtgatggtgatgtcctcgagatctaagcttggatccgcggccgctacgtagaattcc

SEQ ID NO: 42 - DNA, 29 bp, Artificial Sequence
  Description of Artificial Sequence Synthetic primer sHN159
tccatatgcgctcccttctctgacgccgt

SEQ ID NO: 43 - DNA, 80 bp, Artificial Sequence
  Description of Artificial Sequence Synthetic primer NNde1
tatgggccatcaccatcaccatcacgccatgggaattctacgtagcggccgcggatccaagcttagatctcgaggatgaa

SEQ ID NO: 44 - DNA, 82 bp, Artificial Sequence
  Description of Artificial Sequence Synthetic primer NNde2
ctagttcatcctcgagatctaagcttggatccgcggccgctacgtagaattcccatggcgtgatggtgatggtgatggccca

SEQ ID NO: 45 - DNA, 71 bp, Artificial Sequence
  Description of Artificial Sequence Synthetic primer CNde1
tatgggaattctacgtagcggccgcggatccaagcttagatctcgaggacatcaccatcaccatcactgaa

SEQ ID NO: 46 - DNA, 73 bp, Artificial Sequence
  Description of Artificial Sequence Synthetic primer CNde2
ctagttcagtgatggtgatggtgatgtcctcgagatctaagcttggatccgcggccgctacgtagaattccca

SEQ ID NO: 47 - DNA, 32 bp, Artificial Sequence
  Description of Artificial Sequence Synthetic primer sHN160
aacatatgtatatctccttcttaaagttaaac

SEQ ID NO: 48 - DNA, 22 bp, Artificial Sequence
  Description of Artificial Sequence Synthetic primer sHN97
ataccatggaacctcatgaagc

SEQ ID NO: 49 - DNA, 30 bp, Artificial Sequence
  Description of Artificial Sequence Synthetic primer sHN98
aactcgagatcccataagtgctttcatctt

SEQ ID NO: 50 - DNA, 27 bp, Artificial Sequence
  Description of Artificial Sequence Synthetic primer sHN82
tactcatgatgcatcaccatcaccatc

SEQ ID NO: 51 - DNA, 20 bp, Artificial Sequence
  Description of Artificial Sequence Synthetic primer pTrc99A Cseq
cagaccgcttctgcgttctg

SEQ ID NO: 52 - DNA, 27 bp, Artificial Sequence
  Description of Artificial Sequence Synthetic primer sHN272
atccatggcccctatactaggttattg

SEQ ID NO: 53 - DNA, 33 bp, Artificial Sequence
  Description of Artificial Sequence Synthetic primer sHN271
aactcgagtcaatccgattttggaggatggtcg

SEQ ID NO: 54 - DNA, 24 bp, Artificial Sequence
  Description of Artificial Sequence Synthetic primer sHN150
catgggaattcagatctctcgaga

SEQ ID NO: 55 - DNA, 24 bp, Artificial Sequence
  Description of Artificial Sequence Synthetic primer sHN212
agcttctcgagagatctgaattcc

SEQ ID NO: 56 - DNA, 22 bp, Artificial Sequence
  Description of Artificial Sequence Synthetic primer pBAD(Forward)
ctatgccatagcatttttatcc

SEQ ID NO: 57 - DNA, 30 bp, Artificial Sequence
  Description of Artificial Sequence Synthetic primer sHN166
gccatatggggtttttttcatttgttcacg

SEQ ID NO: 58 - DNA, 29 bp, Artificial Sequence
  Description of Artificial Sequence Synthetic primer sHN167
aactcgagtcagtatttgtcaggcagtcc

SEQ ID NO: 59 - DNA, 30 bp, Artificial Sequence
  Description of Artificial Sequence Synthetic primer sHN168
gcctcgaggggtttttttcatttgttcacg

SEQ ID NO: 60 - DNA, 29 bp, Artificial Sequence
  Description of Artificial Sequence Synthetic primer sHN169
gaggtacctcagtatttgtcaggcagtcc

SEQ ID NO: 61 - DNA, 25 bp, Artificial Sequence
  Description of Artificial Sequence Synthetic primer sHN170
cacatatgctccgccagatcctcgg

SEQ ID NO: 62 - DNA, 30 bp, Artificial Sequence
  Description of Artificial Sequence Synthetic primer sHN171
ttgaattcttagaagtctgggccttctttc

SEQ ID NO: 63 - DNA, 28 bp, Artificial Sequence
  Description of Artificial Sequence Synthetic primer sHN172
ccctcgagatgctccgccagatcctcgg

SEQ ID NO: 64 - DNA, 26 bp, Artificial Sequence
  Description of Artificial Sequence Synthetic primer sHN177
cccatatggccgggcagtcagacaag

SEQ ID NO: 65 - DNA, 30 bp, Artificial Sequence
  Description of Artificial Sequence Synthetic primer sHN178
gagaattctcaatcttctgccatgtagagg

SEQ ID NO: 66 - DNA, 29 bp, Artificial Sequence
  Description of Artificial Sequence Synthetic primer sHN179
aactcgagatggccgggcagtcagacaag

SEQ ID NO: 67 - DNA, 30 bp, Artificial Sequence
  Description of Artificial Sequence Synthetic primer sHN290
aacatatgaacaagagctctgaagatatcc

SEQ ID NO: 68 - DNA, 26 bp, Artificial Sequence
  Description of Artificial Sequence Synthetic primer sHN262
atgaattcatggcaaccatctaactg

SEQ ID NO: 69 - DNA, 31 bp, Artificial Sequence
  Description of Artificial Sequence Synthetic primer sHN261
ttctcgagaacaagagctctgaagatatccg

SEQ ID NO: 70 - DNA, 29 bp, Artificial Sequence
  Description of Artificial Sequence Synthetic primer sHN295
aacatatggctgtccctgacaaaacggtc

SEQ ID NO: 71 - DNA, 28 bp, Artificial Sequence
  Description of Artificial Sequence Synthetic primer sHN266
ctaagcttttaatgtttgtggaaagtgc

SEQ ID NO: 72 - DNA, 31 bp, Artificial Sequence
  Description of Artificial Sequence Synthetic primer sHN265
gtctcgaggtccctgacaaaacggtcaaatg

SEQ ID NO: 73 - DNA, 26 bp, Artificial Sequence
  Description of Artificial Sequence Synthetic primer sHN288
ttccatggcacggaagagcctctggg

SEQ ID NO: 74 - DNA, 27 bp, Artificial Sequence
  Description of Artificial Sequence Synthetic primer sHN268
ttgaattccagacaatgagctggaggg

SEQ ID NO: 75 - DNA, 29 bp, Artificial Sequence
  Description of Artificial Sequence Synthetic primer sHN267
aactcgagcggaagagcctctgggactac

SEQ ID NO: 76 - DNA, 31 bp, Artificial Sequence
  Description of Artificial Sequence Synthetic primer sHN243
ctccatggggattatcagaatccctctgcgc

SEQ ID NO: 77 - DNA, 33 bp, Artificial Sequence
  Description of Artificial Sequence Synthetic primer sHN244
agctcgagagagtacgacagcattggcaaagcc

SEQ ID NO: 78 - DNA, 34 bp, Artificial Sequence
  Description of Artificial Sequence Synthetic primer sHN245
aaccatgggcaccaccgatgcggagttccacacc

SEQ ID NO: 79 - DNA, 40 bp, Artificial Sequence
  Description of Artificial Sequence Synthetic primer sHN246
aactcgagatccaaattgatcaatgactttctgtatccac

SEQ ID NO: 80 - DNA, 34 bp, Artificial Sequence
  Description of Artificial Sequence Synthetic primer sHN247
aaccatgggaattgttggaggatttaactgtgag

SEQ ID NO: 81 - DNA, 38 bp, Artificial Sequence
  Description of Artificial Sequence Synthetic primer sHN248
aactcgagagtcattttcagccatagtttctcttatcc

SEQ ID NO: 82 - DNA, 35 bp, Artificial Sequence
  Description of Artificial Sequence Synthetic primer sHN275
aacatatgctgaggctctgctccttcaatgtgagg

SEQ ID NO: 83 - DNA, 25 bp, Artificial Sequence
  Description of Artificial Sequence Synthetic primer sHN307
ttctcgagcgtgatacctaggagcg

SEQ ID NO: 84 - DNA, 28 bp, Artificial Sequence
  Description of Artificial Sequence Synthetic primer sHN277
ggccatggggacaccagaaatctcatgc

SEQ ID NO: 85 - DNA, 30 bp, Artificial Sequence
  Description of Artificial Sequence Synthetic primer sHN278
aagaattcaccgagtttacttacagaaccc

SEQ ID NO: 86 - DNA, 28 bp, Artificial Sequence
  Description of Artificial Sequence Synthetic primer sHN279R
aaccatgggcaaaggagatcctaagaag

SEQ ID NO: 87 - DNA, 33 bp, Artificial Sequence
  Description of Artificial Sequence Synthetic primer sHN280
ttgaattcctgcgctagaaccaacttattcatc

SEQ ID NO: 88 - DNA, 29 bp, Artificial Sequence
  Description of Artificial Sequence Synthetic primer sHN314
aactcgagggcaaaggagatcctaagaag

SEQ ID NO: 89 - DNA, 26 bp, Artificial Sequence
  Description of Artificial Sequence Synthetic primer sHN283
gaccatggctcctgagcaatgggaag

SEQ ID NO: 90 - DNA, 31 bp, Artificial Sequence
  Description of Artificial Sequence Synthetic primer sHN284
ataagcttttaagggtcctcatccacgtgaa

SEQ ID NO: 91 - DNA, 26 bp, Artificial Sequence
  Description of Artificial Sequence Synthetic primer sHN164
aacatatggacgggtccggggagcag

SEQ ID NO: 92 - DNA, 30 bp, Artificial Sequence
  Description of Artificial Sequence Synthetic primer sHN194
aagaattctcagcccatcttcttccagatg

SEQ ID NO: 93 - DNA, 28 bp, Artificial Sequence
  Description of Artificial Sequence Synthetic primer sHN193
aactcgagatggacgggtccggggagca

SEQ ID NO: 94 - DNA, 26 bp, Artificial Sequence
  Description of Artificial Sequence Synthetic primer sHN305
ctcatatggctgtggatactacaagg

SEQ ID NO: 95 - DNA, 29 bp, Artificial Sequence
  Description of Artificial Sequence Synthetic primer sHN306
atctcgaggatttcactggcccagcatgc

SEQ ID NO: 96 - DNA, 30 bp, Artificial Sequence
  Description of Artificial Sequence Synthetic primer sHN296
aactcgagcgtcggtatcctttttgcgctg

SEQ ID NO: 97 - DNA, 27 bp, Artificial Sequence
  Description of Artificial Sequence Synthetic primer sHN330
acccatgggcgacggtgctggaaattg

SEQ ID NO: 98 - DNA, 32 bp, Artificial Sequence
  Description of Artificial Sequence Synthetic primer sHN259
aactcgagatgaagcttgtaaatggcagaaag

SEQ ID NO: 99 - DNA, 28 bp, Artificial Sequence
  Description of Artificial Sequence Synthetic primer sHN260
aagaattcctctactgtgtatcggtcat

SEQ ID NO: 100 - DNA, 30 bp, Artificial Sequence
  Description of Artificial Sequence Synthetic primer sHN269
aactcgagctgcaaggcttggagagtgatg

SEQ ID NO: 101 - DNA, 32 bp, Artificial Sequence
  Description of Artificial Sequence Synthetic primer sHN270
gaggtacctttcagtttagcttgtcgaaatac

SEQ ID NO: 102 - DNA, 31 bp, Artificial Sequence
  Description of Artificial Sequence Synthetic primer sHN263
gcctcgagcttcctgagaagaccatacgatg

SEQ ID NO: 103 - DNA, 30 bp, Artificial Sequence
  Description of Artificial Sequence Synthetic primer sHN264
ctgaattctgtttaatatttatgaaatgtg

SEQ ID NO: 104 - DNA, 41 bp, Artificial Sequence
  Description of Artificial Sequence Synthetic primer sHN343
aaactagttcagtgatggtgatggtgatgctcgagagatct

SEQ ID NO: 105 - DNA, 20 bp, Artificial Sequence
  Description of Artificial Sequence Synthetic primer T7 Universal
taatacgactcactataggg

SEQ ID NO: 106 - DNA, 8166 bp, Artificial Sequence
  Description of Artificial Sequence Synthetic vector pTip NH1
gagctcgaccgcgcgggtcccggacggggaagagcggggagctttgccagagagcgacgacttccccttgcgttggtgattgccggtcagggcagccatccgccatcgtcgcgtagggtgtcacaccccaggaatcgcgtcactgaacacagcagccggtaggacgaccatgactgagttggacaccatcgcaaatccgtccgatcccgcggtgcagcggatcatcgatgtcaccaagccgtcacgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtggacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataagcgcctctgttcctcggacgctcggttcctcgacctcgattcgtcagtgatgatcacctcacacggcagcgatcaccactgacatatcgaggtcaacggtcgtggtccgggcgggcactcctcgaaggcgcggccgacgcccttgaacgactcgatgactctagagtaacgggctactccgtttaacggaccccgttctcacgctttaggcttgaccccggagcctgcatggggcattccgccgtgaacccggtggaatgcccccggcacccgggctttccagcaaagatcacctggcgccgatgagtaaggcgtacagaaccactccacaggaggaccgtcgagatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgtccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggactagaattgatctcctcgaccgccaattgggcatctgagaatcatctgcgtttctcgcacgcaacgtacttgcaacgttgcaactcctagtgttgtgaatcacaccccaccggggggtgggattgcagtcaccgatttggtgggtgcgcccaggaagatcacgtttacataggagcttgcaatgagctactccgtgggacaggtggccggcttcgccggagtgacggtgcgcacgctgcaccactacgacgacatcggcctgctcgtaccgagcgagcgcagccacgcgggccaccggcgctacagcgacgccgacctcgaccggctgcagcagatcctgttctaccgggagctgggcttcccgctcgacgaggtcgccgccctgctcgacgacccggccgcggacccgcgcgcgcacctgcgccgccagcacgagctgctgtccgcccggatcgggaaactgcagaagatggcggcggccgtggagcaggcgatggaggcacgcagcatgggaatcaacctcaccccggaggagaagttcgaggtcttcggcgacttcgaccccgaccagtacgaggaggaggtccgggaacgctgggggaacaccgacgcctaccgccagtccaaggagaagaccgcctcgtacaccaaggaggactggcagcgcatccaggacgaggccgacgagctcacccggcgcttcgtcgccctgatggacgcgggtgagcccgccgactccgagggggcgatggacgccgccgaggaccaccggcagggcatcgcccgcaaccactacgactgcgggtacgagatgcacacctgcctgggcgagatgtacgtgtccgacgaacgtttcacgcgaaacatcgacgccgccaagccgggcctcgccgcctacatgcgcgacgcgatcctcgccaacgccgtccggcacaccccctgagcggtggtcgtggcccgggtctcccgcccggtctcaccccacggctcactcccgggccacgaccaccgccgtcccgtacgcgcacacctcggtgcccacgtccgccgcctccgtcacgtcgaaacggaagatccccgggtaccgagctcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgactagagtcccgctgaggcggcgtagcaggtcagccgccccagcggtggtcaccaaccggggtggaacggcgccggtatcgggtgtgtccgtggcgctcattccaacctccgtgtgtttgtgcaggtttcgcgtgttgcagtccctcgcaccggcacccgcagcgaggggctcacgggtgccggtgggtcgactagttcatcctcgagatctaagcttggatccgcggccgctacgtagaattcccatatggtgatggtgatggtggcccatggccgctcccttctctgacgccgtccacgctgcctcctcacgtgacgtgaggtgcaagcccggacgttccgcgtgccacgccgtgagccgccgcgtgccgtcggctccctcagcccgggcggccgtgggagcccgcctcgatatgtacacccgagaagctcccagcgtcctcctgggccgcgatactcgaccaccacgcacgcacaccgcactaacgattcggccggcgctcgattcggccggcgctcgattcggccggcgctcgattcggccggcgctcgattcggccggcgctcgattcggccgagcagaagagtgaacaaccaccgaccacgcttccgctctgcgcgccgtacccgacctacctcccgcagctcgaagcagctcccgggagtaccgccgtactcacccgcctgtgctcaccatccaccgacgcaaagcccaacccgagcacacctcttgcaccaaggtgccgaccgtggctttccgctcgcagggttccagaagaaatcgaacgatccagcgcggcaaggttcaaaaagcaggggttggtggggaggaggttttggggggtgtcgccgggatacctgatatggctttgttttgcgtagtcgaataattttccatatagcctcggcgcgtcggactcgaatagttgatgtgggcgggcacagttgccccatgaaatccgcaacggggggcgtgctgagcgatcggcaatgggcggatgcggtgttgcttccgcaccggccgttcgcgacgaacaacctccaacgaggtcagtaccggatgagccgcgacgacgcattggcaatgcggtacgtcgagcattcaccgcacgcgttgctcggatctatcgtcatcgactgcgatcacgttgacgccgcgatgcgcgcattcgagcaaccatccgaccatccggcgccgaactgggtcgcacaatcgccgtccggccgcgcacacatcggatggtggctcggccccaaccacgtgtgccgcaccgacagcgcccgactgacgccactgcgctacgcccaccgcatcgaaaccggcctcaagatcagcgtcggcggcgatttcgcgtatggcgggcaactgaccaaaaacccgattcaccccgattgggagacgatctacggcccggccaccccgtacacattgcggcagctggccaccatccacacaccccggcagatgccgcgtcggcccgatcgggccgtgggcctgggccgcaacgtcaccatgttcgacgccacccggcgatgggcatacccgcagtggtggcaacaccgaaacggaaccggccgcgactgggaccatctcgtcctgcagcactgccacgccgtcaacaccgagttcacgacaccactgccgttcaccgaagtacgcgccaccgcgcaatccatctccaaatggatctggcgcaatttcaccgaagaacagtaccgagcccgacaagcgcatctcggtcaaaaaggcggcaaggcaacgacactcgccaaacaagaagccgtccgaaacaatgcaagaaagtacgacgaacatacgatgcgagaggcgattatctgatgggcggagccaaaaatccggtgcgccgaaagatgacggcagcagcagcagccgaaaaattcggtgcctccactcgcacaatccaacgcttgtttgctgagccgcgtgacgattacctcggccgtgcgaaagctcgccgtgacaaagctgtcgagctgcggaagcaggggttgaagtaccgggaaatcgccgaagcgatggaactctcgaccgggatcgtcggccgattactgcacgacgcccgcaggcacggcgagatttcagcggaggatctgtcggcgtaaccaagtcagcgggttgtcgggttccggccggcgctcggcactcggaccggccggcggatggtgttctgcctctggcgcagcgtcagctaccgccgaaggcctgtcatcgaccggcttcgactgaagtatgagcaacgtcacagcctgtgattggatgatccgctcacgctcgaccgctacctgttcagctgccgcccgctgggcatgagcaacggccaactctcgttcaa

SEQ ID NO: 107 - DNA, 8169 bp, Artificial Sequence
  Description of Artificial Sequence Synthetic vector pTip NH2
gagctcgaccgcgcgggtcccggacggggaagagcggggagctttgccagagagcgacgacttccccttgcgttggtgattgccggtcagggcagccatccgccatcgtcgcgtagggtgtcacaccccaggaatcgcgtcactgaacacagcagccggtaggacgaccatgactgagttggacaccatcgcaaatccgtccgatcccgcggtgcagcggatcatcgatgtcaccaagccgtcacgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtggacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataagcgcctctgttcctcggacgctcggttcctcgacctcgattcgtcagtgatgatcacctcacacggcagcgatcaccactgacatatcgaggtcaacggtcgtggtccgggcgggcactcctcgaaggcgcggccgacgcccttgaacgactcgatgactctagagtaacgggctactccgtttaacggaccccgttctcacgctttaggcttgaccccggagcctgcatggggcattccgccgtgaacccggtggaatgcccccggcacccgggctttccagcaaagatcacctggcgccgatgagtaaggcgtacagaaccactccacaggaggaccgtcgagatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgtccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggactagaattgatctcctcgaccgccaattgggcatctgagaatcatctgcgtttctcgcacgcaacgtacttgcaacgttgcaactcctagtgttgtgaatcacaccccaccggggggtgggattgcagtcaccgatttggtgggtgcgcccaggaagatcacgtttacataggagcttgcaatgagctactccgtgggacaggtggccggcttcgccggagtgacggtgcgcacgctgcaccactacgacgacatcggcctgctcgtaccgagcgagcgcagccacgcgggccaccggcgctacagcgacgccgacctcgaccggctgcagcagatcctgttctaccgggagctgggcttcccgctcgacgaggtcgccgccctgctcgacgacccggccgcggacccgcgcgcgcacctgcgccgccagcacgagctgctgtccgcccggatcgggaaactgcagaagatggcggcggccgtggagcaggcgatggaggcacgcagcatgggaatcaacctcaccccggaggagaagttcgaggtcttcggcgacttcgaccccgaccagtacgaggaggaggtccgggaacgctgggggaacaccgacgcctaccgccagtccaaggagaagaccgcctcgtacaccaaggaggactggcagcgcatccaggacgaggccgacgagctcacccggcgcttcgtcgccctgatggacgcgggtgagcccgccgactccgagggggcgatggacgccgccgaggaccaccggcagggcatcgcccgcaaccactacgactgcgggtacgagatgcacacctgcctgggcgagatgtacgtgtccgacgaacgtttcacgcgaaacatcgacgccgccaagccgggcctcgccgcctacatgcgcgacgcgatcctcgccaacgccgtccggcacaccccctgagcggtggtcgtggcccgggtctcccgcccggtctcaccccacggctcactcccgggccacgaccaccgccgtcccgtacgcgcacacctcggtgcccacgtccgccgcctccgtcacgtcgaaacggaagatccccgggtaccgagctcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgactagagtcccgctgaggcggcgtagcaggtcagccgccccagcggtggtcaccaaccggggtggaacggcgccggtatcgggtgtgtccgtggcgctcattccaacctccgtgtgtttgtgcaggtttcgcgtgttgcagtccctcgcaccggcacccgcagcgaggggctcacgggtgccggtgggtcgactagttcatcctcgagatctaagcttggatccgcggccgctacgtagaattcccatggcgtgatggtgatggtgatggcccatatgcgctcccttctctgacgccgtccacgctgcctcctcacgtgacgtgaggtgcaagcccggacgttccgcgtgccacgccgtgagccgccgcgtgccgtcggctccctcagcccgggcggccgtgggagcccgcctcgatatgtacacccgagaagctcccagcgtcctcctgggccgcgatactcgaccaccacgcacgcacaccgcactaacgattcggccggcgctcgattcggccggcgctcgattcggccggcgctcgattcggccggcgctcgattcggccggcgctcgattcggccgagcagaagagtgaacaaccaccgaccacgcttccgctctgcgcgccgtacccgacctacctcccgcagctcgaagcagctcccgggagtaccgccgtactcacccgcctgtgctcaccatccaccgacgcaaagcccaacccgagcacacctcttgcaccaaggtgccgaccgtggctttccgctcgcagggttccagaagaaatcgaacgatccagcgcggcaaggttcaaaaagcaggggttggtggggaggaggttttggggggtgtcgccgggatacctgatatggctttgttttgcgtagtcgaataattttccatatagcctcggcgcgtcggactcgaatagttgatgtgggcgggcacagttgccccatgaaatccgcaacggggggcgtgctgagcgatcggcaatgggcggatgcggtgttgcttccgcaccggccgttcgcgacgaacaacctccaacgaggtcagtaccggatgagccgcgacgacgcattggcaatgcggtacgtcgagcattcaccgcacgcgttgctcggatctatcgtcatcgactgcgatcacgttgacgccgcgatgcgcgcattcgagcaaccatccgaccatccggcgccgaactgggtcgcacaatcgccgtccggccgcgcacacatcggatggtggctcggccccaaccacgtgtgccgcaccgacagcgcccgactgacgccactgcgctacgcccaccgcatcgaaaccggcctcaagatcagcgtcggcggcgatttcgcgtatggcgggcaactgaccaaaaacccgattcaccccgattgggagacgatctacggcccggccaccccgtacacattgcggcagctggccaccatccacacaccccggcagatgccgcgtcggcccgatcgggccgtgggcctgggccgcaacgtcaccatgttcgacgccacccggcgatgggcatacccgcagtggtggcaacaccgaaacggaaccggccgcgactgggaccatctcgtcctgcagcactgccacgccgtcaacaccgagttcacgacaccactgccgttcaccgaagtacgcgccaccgcgcaatccatctccaaatggatctggcgcaatttcaccgaagaacagtaccgagcccgacaagcgcatctcggtcaaaaaggcggcaaggcaacgacactcgccaaacaagaagccgtccgaaacaatgcaagaaagtacgacgaacatacgatgcgagaggcgattatctgatgggcggagccaaaaatccggtgcgccgaaagatgacggcagcagcagcagccgaaaaattcggtgcctccactcgcacaatccaacgcttgtttgctgagccgcgtgacgattacctcggccgtgcgaaagctcgccgtgacaaagctgtcgagctgcggaagcaggggttgaagtaccgggaaatcgccgaagcgatggaactctcgaccgggatcgtcggccgattactgcacgacgcccgcaggcacggcgagatttcagcggaggatctgtcggcgtaaccaagtcagcgggttgtcgggttccggccggcgctcggcactcggaccggccggcggatggtgttctgcctctggcgcagcgtcagctaccgccgaaggcctgtcatcgaccggcttcgactgaagtatgagcaacgtcacagcctgtgattggatgatccgctcacgctcgaccgctacctgttcagctgccgcccgctgggcatgagcaacggccaactctcgttcaa

SEQ ID NO: 108 - DNA, 8160 bp, Artificial Sequence
  Description of Artificial Sequence Synthetic vector pTip CH1
gagctcgaccgcgcgggtcccggacggggaagagcggggagctttgccagagagcgacgacttccccttgcgttggtgattgccggtcagggcagccatccgccatcgtcgcgtagggtgtcacaccccaggaatcgcgtcactgaacacagcagccggtaggacgaccatgactgagttggacaccatcgcaaatccgtccgatcccgcggtgcagcggatcatcgatgtcaccaagccgtcacgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtggacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataagcgcctctgttcctcggacgctcggttcctcgacctcgattcgtcagtgatgatcacctcacacggcagcgatcaccactgacatatcgaggtcaacggtcgtggtccgggcgggcactcctcgaaggcgcggccgacgcccttgaacgactcgatgactctagagtaacgggctactccgtttaacggaccccgttctcacgctttaggcttgaccccggagcctgcatggggcattccgccgtgaacccggtggaatgcccccggcacccgggctttccagcaaagatcacctggcgccgatgagtaaggcgtacagaaccactccacaggaggaccgtcgagatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgtccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggactagaattgatctcctcgaccgccaattgggcatctgagaatcatctgcgtttctcgcacgcaacgtacttgcaacgttgcaactcctagtgttgtgaatcacaccccaccggggggtgggattgcagtcaccgatttggtgggtgcgcccaggaagatcacgtttacataggagcttgcaatgagctactccgtgggacaggtggccggcttcgccggagtgacggtgcgcacgctgcaccactacgacgacatcggcctgctcgtaccgagcgagcgcagccacgcgggccaccggcgctacagcgacgccgacctcgaccggctgcagcagatcctgttctaccgggagctgggcttcccgctcgacgaggtcgccgccctgctcgacgacccggccgcggacccgcgcgcgcacctgcgccgccagcacgagctgctgtccgcccggatcgggaaactgcagaagatggcggcggccgtggagcaggcgatggaggcacgcagcatgggaatcaacctcaccccggaggagaagttcgaggtcttcggcgacttcgaccccgaccagtacgaggaggaggtccgggaacgctgggggaacaccgacgcctaccgccagtccaaggagaagaccgcctcgtacaccaaggaggactggcagcgcatccaggacgaggccgacgagctcacccggcgcttcgtcgccctgatggacgcgggtgagcccgccgactccgagggggcgatggacgccgccgaggaccaccggcagggcatcgcccgcaaccactacgactgcgggtacgagatgcacacctgcctgggcgagatgtacgtgtccgacgaacgtttcacgcgaaacatcgacgccgccaagccgggcctcgccgcctacatgcgcgacgcgatcctcgccaacgccgtccggcacaccccctgagcggtggtcgtggcccgggtctcccgcccggtctcaccccacggctcactcccgggccacgaccaccgccgtcccgtacgcgcacacctcggtgcccacgtccgccgcctccgtcacgtcgaaacggaagatccccgggtaccgagctcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgactagagtcccgctgaggcggcgtagcaggtcagccgccccagcggtggtcaccaaccggggtggaacggcgccggtatcgggtgtgtccgtggcgctcattccaacctccgtgtgtttgtgcaggtttcgcgtgttgcagtccctcgcaccggcacccgcagcgaggggctcacgggtgccggtgggtcgactagttcagtgatggtgatggtgatgtcctcgagatctaagcttggatccgcggccgctacgtagaattcccatggccgctcccttctctgacgccgtccacgctgcctcctcacgtgacgtgaggtgcaagcccggacgttccgcgtgccacgccgtgagccgccgcgtgccgtcggctccctcagcccgggcggccgtgggagcccgcctcgatatgtacacccgagaagctcccagcgtcctcctgggccgcgatactcgaccaccacgcacgcacaccgcactaacgattcggccggcgctcgattcggccggcgctcgattcggccggcgctcgattcggccggcgctcgattcggccggcgctcgattcggccgagcagaagagtgaacaaccaccgaccacgcttccgctctgcgcgccgtacccgacctacctcccgcagctcgaagcagctcccgggagtaccgccgtactcacccgcctgtgctcaccatccaccgacgcaaagcccaacccgagcacacctcttgcaccaaggtgccgaccgtggctttccgctcgcagggttccagaagaaatcgaacgatccagcgcggcaaggttcaaaaagcaggggttggtggggaggaggttttggggggtgtcgccgggatacctgatatggctttgttttgcgtagtcgaataattttccatatagcctcggcgcgtcggactcgaatagttgatgtgggcgggcacagttgccccatgaaatccgcaacggggggcgtgctgagcgatcggcaatgggcggatgcggtgttgcttccgcaccggccgttcgcgacgaacaacctccaacgaggtcagtaccggatgagccgcgacgacgcattggcaatgcggtacgtcgagcattcaccgcacgcgttgctcggatctatcgtcatcgactgcgatcacgttgacgccgcgatgcgcgcattcgagcaaccatccgaccatccggcgccgaactgggtcgcacaatcgccgtccggccgcgcacacatcggatggtggctcggccccaaccacgtgtgccgcaccgacagcgcccgactgacgccactgcgctacgcccaccgcatcgaaaccggcctcaagatcagcgtcggcggcgatttcgcgtatggcgggcaactgaccaaaaacccgattcaccccgattgggagacgatctacggcccggccaccccgtacacattgcggcagctggccaccatccacacaccccggcagatgccgcgtcggcccgatcgggccgtgggcctgggccgcaacgtcaccatgttcgacgccacccggcgatgggcatacccgcagtggtggcaacaccgaaacggaaccggccgcgactgggaccatctcgtcctgcagcactgccacgccgtcaacaccgagttcacgacaccactgccgttcaccgaagtacgcgccaccgcgcaatccatctccaaatggatctggcgcaatttcaccgaagaacagtaccgagcccgacaagcgcatctcggtcaaaaaggcggcaaggcaacgacactcgccaaacaagaagccgtccgaaacaatgcaagaaagtacgacgaacatacgatgcgagaggcgattatctgatgggcggagccaaaaatccggtgcgccgaaagatgacggcagcagcagcagccgaaaaattcggtgcctccactcgcacaatccaacgcttgtttgctgagccgcgtgacgattacctcggccgtgcgaaagctcgccgtgacaaagctgtcgagctgcggaagcaggggttgaagtaccgggaaatcgccgaagcgatggaactctcgaccgggatcgtcggccgattactgcacgacgcccgcaggcacggcgagatttcagcggaggatctgtcggcgtaaccaagtcagcgggttgtcgggttccggccggcgctcggcactcggaccggccggcggatggtgttctgcctctggcgcagcgtcagctaccgccgaaggcctgtcatcgaccggcttcgactgaagtatgagcaacgtcacagcctgtgattggatgatccgctcacgctcgaccgctacctgttcagctgccgcccgctgggcatgagcaacggccaactctcgttcaa

SEQ ID NO: 109 - DNA, 8160 bp, Artificial Sequence
  Description of Artificial Sequence Synthetic vector pTip CH2
gagctcgaccgcgcgggtcccggacggggaagagcggggagctttgccagagagcgacgacttccccttgcgttggtgattgccggtcagggcagccatccgccatcgtcgcgtagggtgtcacaccccaggaatcgcgtcactgaacacagcagccggtaggacgaccatgactgagttggacaccatcgcaaatccgtccgatcccgcggtgcagcggatcatcgatgtcaccaagccgtcacgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtggacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataagcgcctctgttcctcggacgctcggttcctcgacctcgattcgtcagtgatgatcacctcacacggcagcgatcaccactgacatatcgaggtcaacggtcgtggtccgggcgggcactcctcgaaggcgcggccgacgcccttgaacgactcgatgactctagagtaacgggctactccgtttaacggaccccgttctcacgctttaggcttgaccccggagcctgcatggggcattccgccgtgaacccggtggaatgcccccggcacccgggctttccagcaaagatcacctggcgccgatgagtaaggcgtacagaaccactccacaggaggaccgtcgagatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgtccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggactagaattgatctcctcgaccgccaattgggcatctgagaatcatctgcgtttctcgcacgcaacgtacttgcaacgttgcaactcctagtgttgtgaatcacaccccaccggggggtgggattgcagtcaccgatttggtgggtgcgcccaggaagatcacgtttacataggagcttgcaatgagctactccgtgggacaggtggccggcttcgccggagtgacggtgcgcacgctgcaccactacgacgacatcggcctgctcgtaccgagcgagcgcagccacgcgggccaccggcgctacagcgacgccgacctcgaccggctgcagcagatcctgttctaccgggagctgggcttcccgctcgacgaggtcgccgccctgctcgacgacccggccgcggacccgcgcgcgcacctgcgccgccagcacgagctgctgtccgcccggatcgggaaactgcagaagatggcggcggccgtggagcaggcgatggaggcacgcagcatgggaatcaacctcaccccggaggagaagttcgaggtcttcggcgacttcgaccccgaccagtacgaggaggaggtccgggaacgctgggggaacaccgacgcctaccgccagtccaaggagaagaccgcctcgtacaccaaggaggactggcagcgcatccaggacgaggccgacgagctcacccggcgcttcgtcgccctgatggacgcgggtgagcccgccgactccgagggggcgatggacgccgccgaggaccaccggcagggcatcgcccgcaaccactacgactgcgggtacgagatgcacacctgcctgggcgagatgtacgtgtccgacgaacgtttcacgcgaaacatcgacgccgccaagccgggcctcgccgcctacatgcgcgacgcgatcctcgccaacgccgtccggcacaccccctgagcggtggtcgtggcccgggtctcccgcccggtctcaccccacggctcactcccgggccacgaccaccgccgtcccgtacgcgcacacctcggtgcccacgtccgccgcctccgtcacgtcgaaacggaagatccccgggtaccgagctcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgactagagtcccgctgaggcggcgtagcaggtcagccgccccagcggtggtcaccaaccggggtggaacggcgccggtatcgggtgtgtccgtggcgctcattccaacctccgtgtgtttgtgcaggtttcgcgtgttgcagtccctcgcaccggcacccgcagcgaggggctcacgggtgccggtgggtcgactagttcagtgatggtgatggtgatgtcctcgagatctaagcttggatccgcggccgctacgtagaattcccatatgcgctcccttctctgacgccgtccacgctgcctcctcacgtgacgtgaggtgcaagcccggacgttccgcgtgccacgccgtgagccgccgcgtgccgtcggctccctcagcccgggcggccgtgggagcccgcctcgatatgtacacccgagaagctcccagcgtcctcctgggccgcgatactcgaccaccacgcacgcacaccgcactaacgattcggccggcgctcgattcggccggcgctcgattcggccggcgctcgattcggccggcgctcgattcggccggcgctcgattcggccgagcagaagagtgaacaaccaccgaccacgcttccgctctgcgcgccgtacccgacctacctcccgcagctcgaagcagctcccgggagtaccgccgtactcacccgcctgtgctcaccatccaccgacgcaaagcccaacccgagcacacctcttgcaccaaggtgccgaccgtggctttccgctcgcagggttccagaagaaatcgaacgatccagcgcggcaaggttcaaaaagcaggggttggtggggaggaggttttggggggtgtcgccgggatacctgatatggctttgttttgcgtagtcgaataattttccatatagcctcggcgcgtcggactcgaatagttgatgtgggcgggcacagttgccccatgaaatccgcaacggggggcgtgctgagcgatcggcaatgggcggatgcggtgttgcttccgcaccggccgttcgcgacgaacaacctccaacgaggtcagtaccggatgagccgcgacgacgcattggcaatgcggtacgtcgagcattcaccgcacgcgttgctcggatctatcgtcatcgactgcgatcacgttgacgccgcgatgcgcgcattcgagcaaccatccgaccatccggcgccgaactgggtcgcacaatcgccgtccggccgcgcacacatcggatggtggctcggccccaaccacgtgtgccgcaccgacagcgcccgactgacgccactgcgctacgcccaccgcatcgaaaccggcctcaagatcagcgtcggcggcgatttcgcgtatggcgggcaactgaccaaaaacccgattcaccccgattgggagacgatctacggcccggccaccccgtacacattgcggcagctggccaccatccacacaccccggcagatgccgcgtcggcccgatcgggccgtgggcctgggccgcaacgtcaccatgttcgacgccacccggcgatgggcatacccgcagtggtggcaacaccgaaacggaaccggccgcgactgggaccatctcgtcctgcagcactgccacgccgtcaacaccgagttcacgacaccactgccgttcaccgaagtacgcgccaccgcgcaatccatctccaaatggatctggcgcaatttcaccgaagaacagtaccgagcccgacaagcgcatctcggtcaaaaaggcggcaaggcaacgacactcgccaaacaagaagccgtccgaaacaatgcaagaaagtacgacgaacatacgatgcgagaggcgattatctgatgggcggagccaaaaatccggtgcgccgaaagatgacggcagcagcagcagccgaaaaattcggtgcctccactcgcacaatccaacgcttgtttgctgagccgcgtgacgattacctcggccgtgcgaaagctcgccgtgacaaagctgtcgagctgcggaagcaggggttgaagtaccgggaaatcgccgaagcgatggaactctcgaccgggatcgtcggccgattactgcacgacgcccgcaggcacggcgagatttcagcggaggatctgtcggcgtaaccaagtcagcgggttgtcgggttccggccggcgctcggcactcggaccggccggcggatggtgttctgcctctggcgcagcgtcagctaccgccgaaggcctgtcatcgaccggcttcgactgaagtatgagcaacgtcacagcctgtgattggatgatccgctcacgctcgaccgctacctgttcagctgccgcccgctgggcatgagcaacggccaactctcgttcaa

SEQ ID NO: 110 - DNA, 8189 bp, Artificial Sequence
  Description of Artificial Sequence Synthetic vector pTip LNH1
gagctcgaccgcgcgggtcccggacggggaagagcggggagctttgccagagagcgacgacttccccttgcgttggtgattgccggtcagggcagccatccgccatcgtcgcgtagggtgtcacaccccaggaatcgcgtcactgaacacagcagccggtaggacgaccatgactgagttggacaccatcgcaaatccgtccgatcccgcggtgcagcggatcatcgatgtcaccaagccgtcacgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtggacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataagcgcctctgttcctcggacgctcggttcctcgacctcgattcgtcagtgatgatcacctcacacggcagcgatcaccactgacatatcgaggtcaacggtcgtggtccgggcgggcactcctcgaaggcgcggccgacgcccttgaacgactcgatgactctagagtaacgggctactccgtttaacggaccccgttctcacgctttaggcttgaccccggagcctgcatggggcattccgccgtgaacccggtggaatgcccccggcacccgggctttccagcaaagatcacctggcgccgatgagtaaggcgtacagaaccactccacaggaggaccgtcgagatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgtccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggactagaattgatctcctcgaccgccaattgggcatctgagaatcatctgcgtttctcgcacgcaacgtacttgcaacgttgcaactcctagtgttgtgaatcacaccccaccggggggtgggattgcagtcaccgatttggtgggtgcgcccaggaagatcacgtttacataggagcttgcaatgagctactccgtgggacaggtggccggcttcgccggagtgacggtgcgcacgctgcaccactacgacgacatcggcctgctcgtaccgagcgagcgcagccacgcgggccaccggcgctacagcgacgccgacctcgaccggctgcagcagatcctgttctaccgggagctgggcttcccgctcgacgaggtcgccgccctgctcgacgacccggccgcggacccgcgcgcgcacctgcgccgccagcacgagctgctgtccgcccggatcgggaaactgcagaagatggcggcggccgtggagcaggcgatggaggcacgcagcatgggaatcaacctcaccccggaggagaagttcgaggtcttcggcgacttcgaccccgaccagtacgaggaggaggtccgggaacgctgggggaacaccgacgcctaccgccagtccaaggagaagaccgcctcgtacaccaaggaggactggcagcgcatccaggacgaggccgacgagctcacccggcgcttcgtcgccctgatggacgcgggtgagcccgccgactccgagggggcgatggacgccgccgaggaccaccggcagggcatcgcccgcaaccactacgactgcgggtacgagatgcacacctgcctgggcgagatgtacgtgtccgacgaacgtttcacgcgaaacatcgacgccgccaagccgggcctcgccgcctacatgcgcgacgcgatcctcgccaacgccgtccggcacaccccctgagcggtggtcgtggcccgggtctcccgcccggtctcaccccacggctcactcccgggccacgaccaccgccgtcccgtacgcgcacacctcggtgcccacgtccgccgcctccgtcacgtcgaaacggaagatccccgggtaccgagctcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgactagagtcccgctgaggcggcgtagcaggtcagccgccccagcggtggtcaccaaccggggtggaacggcgccggtatcgggtgtgtccgtggcgctcattccaacctccgtgtgtttgtgcaggtttcgcgtgttgcagtccctcgcaccggcacccgcagcgaggggctcacgggtgccggtgggtcgactagttcatcctcgagatctaagcttggatccgcggccgctacgtagaattcccatatggtgatggtgatggtggcccatggtatatctccttcttaaagttaaacaaaattatttctagacgccgtccacgctgcctcctcacgtgacgtgaggtgcaagcccggacgttccgcgtgccacgccgtgagccgccgcgtgccgtcggctccctcagcccgggcggccgtgggagcccgcctcgatatgtacacccgagaagctcccagcgtcctcctgggccgcgatactcgaccaccacgcacgcacaccgcactaacgattcggccggcgctcgattcggccggcgctcgattcggccggcgctcgattcggccggcgctcgattcggccggcgctcgattcggccgagcagaagagtgaacaaccaccgaccacgcttccgctctgcgcgccgtacccgacctacctcccgcagctcgaagcagctcccgggagtaccgccgtactcacccgcctgtgctcaccatccaccgacgcaaagcccaacccgagcacacctcttgcaccaaggtgccgaccgtggctttccgctcgcagggttccagaagaaatcgaacgatccagcgcggcaaggttcaaaaagcaggggttggtggggaggaggttttggggggtgtcgccgggatacctgatatggctttgttttgcgtagtcgaataattttccatatagcctcggcgcgtcggactcgaatagttgatgtgggcgggcacagttgccccatgaaatccgcaacggggggcgtgctgagcgatcggcaatgggcggatgcggtgttgcttccgcaccggccgttcgcgacgaacaacctccaacgaggtcagtaccggatgagccgcgacgacgcattggcaatgcggtacgtcgagcattcaccgcacgcgttgctcggatctatcgtcatcgactgcgatcacgttgacgccgcgatgcgcgcattcgagcaaccatccgaccatccggcgccgaactgggtcgcacaatcgccgtccggccgcgcacacatcggatggtggctcggccccaaccacgtgtgccgcaccgacagcgcccgactgacgccactgcgctacgcccaccgcatcgaaaccggcctcaagatcagcgtcggcggcgatttcgcgtatggcgggcaactgaccaaaaacccgattcaccccgattgggagacgatctacggcccggccaccccgtacacattgcggcagctggccaccatccacacaccccggcagatgccgcgtcggcccgatcgggccgtgggcctgggccgcaacgtcaccatgttcgacgccacccggcgatgggcatacccgcagtggtggcaacaccgaaacggaaccggccgcgactgggaccatctcgtcctgcagcactgccacgccgtcaacaccgagttcacgacaccactgccgttcaccgaagtacgcgccaccgcgcaatccatctccaaatggatctggcgcaatttcaccgaagaacagtaccgagcccgacaagcgcatctcggtcaaaaaggcggcaaggcaacgacactcgccaaacaagaagccgtccgaaacaatgcaagaaagtacgacgaacatacgatgcgagaggcgattatctgatgggcggagccaaaaatccggtgcgccgaaagatgacggcagcagcagcagccgaaaaattcggtgcctccactcgcacaatccaacgcttgtttgctgagccgcgtgacgattacctcggccgtgcgaaagctcgccgtgacaaagctgtcgagctgcggaagcaggggttgaagtaccgggaaatcgccgaagcgatggaactctcgaccgggatcgtcggccgattactgcacgacgcccgcaggcacggcgagatttcagcggaggatctgtcggcgtaaccaagtcagcgggttgtcgggttccggccggcgctcggcactcggaccggccggcggatggtgttctgcctctggcgcagcgtcagctaccgccgaaggcctgtcatcgaccggcttcgactgaagtatgagcaacgtcacagcctgtgattggatgatccgctcacgctcgaccgctacctgttcagctgccgcccgctgggcatgagcaacggccaactctcgttcaa

SEQ ID NO: 111 - DNA, 8183 bp, Artificial Sequence
  Description of Artificial Sequence Synthetic vector pTip LNH2
gagctcgaccgcgcgggtcccggacggggaagagcggggagctttgccagagagcgacgacttccccttgcgttggtgattgccggtcagggcagccatccgccatcgtcgcgtagggtgtcacaccccaggaatcgcgtcactgaacacagcagccggtaggacgaccatgactgagttggacaccatcgcaaatccgtccgatcccgcggtgcagcggatcatcgatgtcaccaagccgtcacgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtggacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataagcgcctctgttcctcggacgctcggttcctcgacctcgattcgtcagtgatgatcacctcacacggcagcgatcaccactgacatatcgaggtcaacggtcgtggtccgggcgggcactcctcgaaggcgcggccgacgcccttgaacgactcgatgactctagagtaacgggctactccgtttaacggaccccgttctcacgctttaggcttgaccccggagcctgcatggggcattccgccgtgaacccggtggaatgcccccggcacccgggctttccagcaaagatcacctggcgccgatgagtaaggcgtacagaaccactccacaggaggaccgtcgagatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgtccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggactagaattgatctcctcgaccgccaattgggcatctgagaatcatctgcgtttctcgcacgcaacgtacttgcaacgttgcaactcctagtgttgtgaatcacaccccaccggggggtgggattgcagtcaccgatttggtgggtgcgcccaggaagatcacgtttacataggagcttgcaatgagctactccgtgggacaggtggccggcttcgccggagtgacggtgcgcacgctgcaccactacgacgacatcggcctgctcgtaccgagcgagcgcagccacgcgggccaccggcgctacagcgacgccgacctcgaccggctgcagcagatcctgttctaccgggagctgggcttcccgctcgacgaggtcgccgccctgctcgacgacccggccgcggacccgcgcgcgcacctgcgccgccagcacgagctgctgtccgcccggatcgggaaactgcagaagatggcggcggccgtggagcaggcgatggaggcacgcagcatgggaatcaacctcaccccggaggagaagttcgaggtcttcggcgacttcgaccccgaccagtacgaggaggaggtccgggaacgctgggggaacaccgacgcctaccgccagtccaaggagaagaccgcctcgtacaccaaggaggactggcagcgcatccaggacgaggccgacgagctcacccggcgcttcgtcgccctgatggacgcgggtgagcccgccgactccgagggggcgatggacgccgccgaggaccaccggcagggcatcgcccgcaaccactacgactgcgggtacgagatgcacacctgcctgggcgagatgtacgtgtccgacgaacgtttcacgcgaaacatcgacgccgccaagccgggcctcgccgcctacatgcgcgacgcgatcctcgccaacgccgtccggcacaccccctgagcggtggtcgtggcccgggtctcccgcccggtctcaccccacggctcactcccgggccacgaccaccgccgtcccgtacgcgcacacctcggtgcccacgtccgccgcctccgtcacgtcgaaacggaagatccccgggtaccgagctcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgactagagtcccgctgaggcggcgtagcaggtcagccgccccagcggtggtcaccaaccggggtggaacggcgccggtatcgggtgtgtccgtggcgctcattccaacctccgtgtgtttgtgcaggtttcgcgtgttgcagtccctcgcaccggcacccgcagcgaggggctcacgggtgccggtgggtcgactagttcagtgatggtgatggtgatgtcctcgagatctaagcttggatccgcggccgctacgtagaattcccatggtatatctccttcttaaagttaaacaaaattatttctagacgccgtccacgctgcctcctcacgtgacgtgaggtgcaagcccggacgttccgcgtgccacgccgtgagccgccgcgtgccgtcggctccctcagcccgggcggccgtgggagcccgcctcgatatgtacacccgagaagctcccagcgtcctcctgggccgcgatactcgaccaccacgcacgcacaccgcactaacgattcggccggcgctcgattcggccggcgctcgattcggccggcgctcgattcggccggcgctcgattcggccggcgctcgattcggccgagcagaagagtgaacaaccaccgaccacgcttccgctctgcgcgccgtacccgacctacctcccgcagctcgaagcagctcccgggagtaccgccgtactcacccgcctgtgctcaccatccaccgacgcaaagcccaacccgagcacacctcttgcaccaaggtgccgaccgtggctttccgctcgcagggttccagaagaaatcgaacgatccagcgcggcaaggttcaaaaagcaggggttggtggggaggaggttttggggggtgtcgccgggatacctgatatggctttgttttgcgtagtcgaataattttccatatagcctcggcgcgtcggactcgaatagttgatgtgggcgggcacagttgccccatgaaatccgcaacggggggcgtgctgagcgatcggcaatgggcggatgcggtgttgcttccgcaccggccgttcgcgacgaacaacctccaacgaggtcagtaccggatgagccgcgacgacgcattggcaatgcggtacgtcgagcattcaccgcacgcgttgctcggatctatcgtcatcgactgcgatcacgttgacgccgcgatgcgcgcattcgagcaaccatccgaccatccggcgccgaactgggtcgcacaatcgccgtccggccgcgcacacatcggatggtggctcggccccaaccacgtgtgccgcaccgacagcgcccgactgacgccactgcgctacgcccaccgcatcgaaaccggcctcaagatcagcgtcggcggcgatttcgcgtatggcgggcaactgaccaaaaacccgattcaccccgattgggagacgatctacggcccggccaccccgtacacattgcggcagctggccaccatccacacaccccggcagatgccgcgtcggcccgatcgggccgtgggcctgggccgcaacgtcaccatgttcgacgccacccggcgatgggcatacccgcagtggtggcaacaccgaaacggaaccggccgcgactgggaccatctcgtcctgcagcactgccacgccgtcaacaccgagttcacgacaccactgccgttcaccgaagtacgcgccaccgcgcaatccatctccaaatggatctggcgcaatttcaccgaagaacagtaccgagcccgacaagcgcatctcggtcaaaaaggcggcaaggcaacgacactcgccaaacaagaagccgtccgaaacaatgcaagaaagtacgacgaacatacgatgcgagaggcgattatctgatgggcggagccaaaaatccggtgcgccgaaagatgacggcagcagcagcagccgaaaaattcggtgcctccactcgcacaatccaacgcttgtttgctgagccgcgtgacgattacctcggccgtgcgaaagctcgccgtgacaaagctgtcgagctgcggaagcaggggttgaagtaccgggaaatcgccgaagcgatggaactctcgaccgggatcgtcggccgattactgcacgacgcccgcaggcacggcgagatttcagcggaggatctgtcggcgtaaccaagtcagcgggttgtcgggttccggccggcgctcggcactcggaccggccggcggatggtgttctgcctctggcgcagcgtcagctaccgccgaaggcctgtcatcgaccggcttcgactgaagtatgagcaacgtcacagcctgtgattggatgatccgctcacgctcgaccgctacctgttcagctgccgcccgctgggcatgagcaacggccaactctcgttcaa

SEQ ID NO: 112 - DNA, 8123 bp, Artificial Sequence
  Description of Artificial Sequence Synthetic vector pTip LCH1
gagctcgaccgcgcgggtcccggacggggaagagcggggagctttgccagagagcgacgacttccccttgcgttggtgattgccggtcagggcagccatccgccatcgtcgcgtagggtgtcacaccccaggaatcgcgtcactgaacacagcagccggtaggacgaccatgactgagttggacaccatcgcaaatccgtccgatcccgcggtgcagcggatcatcgatgtcaccaagccgtcacgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtggacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataagcgcctctgttcctcggacgctcggttcctcgacctcgattcgtcagtgatgatcacctcacacggcagcgatcaccactgacatatcgaggtcaacggtcgtggtccgggcgggcactcctcgaaggcgcggccgacgcccttgaacgactcgatgactctagagtaacgggctactccgtttaacggaccccgttctcacgctttaggcttgaccccggagcctgcatggggcattccgccgtgaacccggtggaatgcccccggcacccgggctttccagcaaagatcacctggcgccgatgagtaaggcgtacagaaccactccacaggaggaccgtcgagatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgtccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggactagaattgatctcctcgaccgccaattgggcatctgagaatcatctgcgtttctcgcacgcaacgtacttgcaacgttgcaactcctagtgttgtgaatcacaccccaccggggggtgggattgcagtcaccgatttggtgggtgcgcccaggaagatcacgtttacataggagcttgcaatgagctactccgtgggacaggtggccggcttcgccggagtgacggtgcgcacgctgcaccactacgacgacatcggcctgctcgtaccgagcgagcgcagccacgcgggccaccggcgctacagcgacgccgacctcgaccggctgcagcagatcctgttctaccgggagctgggcttcccgctcgacgaggtcgccgccctgctcgacgacccggccgcggacccgcgcgcgcacctgcgccgccagcacgagctgctgtccgcccggatcgggaaactgcagaagatggcggcggccgtggagcaggcgatggaggcacgcagcatgggaatcaacctcaccccggaggagaagttcgaggtcttcggcgacttcgaccccgaccagtacgaggaggaggtccgggaacgctgggggaacaccgacgcctaccgccagtccaaggagaagaccgcctcgtacaccaaggaggactggcagcgcatccaggacgaggccgacgagctcacccggcgcttcgtcgccctgatggacgcgggtgagcccgccgactccgagggggcgatggacgccgccgaggaccaccggcagggcatcgcccgcaaccactacgactgcgggtacgagatgcacacctgcctgggcgagatgtacgtgtccgacgaacgtttcacgcgaaacatcgacgccgccaagccgggcctcgccgcctacatgcgcgacgcgatcctcgccaacgccgtccggcacaccccctgagcggtggtcgtggcccgggtctcccgcccggtctcaccccacggctcactcccgggccacgaccaccgccgtcccgtacgcgcacacctcggtgcccacgtccgccgcctccgtcacgtcgaaacggaagatccccgggtaccgagctcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgactagagtcccgctgaggcggcgtagcaggtcagccgccccagcggtggtcaccaaccggggtggaacggcgccggtatcgggtgtgtccgtggcgctcattccaacctccgtgtgtttgtgcaggtttcgcgtgttgcagtccctcgcaccggcacccgcagcgaggggctcacgggtgccggtgggtcgactagttcagtgatggtgatggtgatgtcctcgagatctaagcttggatccgcggccgctacgtagaattcccatggtatatctccttcttaaagttaaacaaaattatttctagacgccgtccacgctgcctcctcacgtgacgtgaggtgcaagcccggacgttccgcgtgccacgccgtgagccgccgcgtgccgtcggctccctcagcccgggcggccgtgggagcccgcctcgatatgtacacccgagaagctcccagcgtcctcctgggccgcgatactcgaccaccacgcacgcacaccgcactaacgattcggccggcgctcgattcggccggcgctcgattcggccggcgctcgattcggccggcgctcgattcggccggcgctcgattcggccgagcagaagagtgaacaaccaccgaccacgcttccgctctgcgcgccgtacccgacctacctcccgcagctcgaagcagctcccgggagtaccgccgtactcacccgcctgtgctcaccatccaccgacgcaaagcccaacccgagcacacctcttgcaccaaggtgccgaccgtggctttccgctcgcagggttccagaagaaatcgaacgatccagcgcggcaaggttcaaaaagcaggggttggtggggaggaggttttggggggtgtcgccgggatacctgatatggctttgttttgcgtagtcgaataattttccatatagcctcggcgcgtcggactcgaatagttgatgtgggcgggcacagttgccccatgaaatccgcaacggggggcgtgctgagcgatcggcaatgggcggatgcggtgttgcttccgcaccggccgttcgcgacgaacaacctccaacgaggtcagtaccggatgagccgcgacgacgcattggcaatgcggtacgtcgagcattcaccgcacgcgttgctcggatctatcgtcatcgactgcgatcacgttgacgccgcgatgcgcgcattcgagcaaccatccgaccatccggcgccgaactgggtcgcacaatcgccgtccggccgcgcacacatcggatggtggctcggccccaaccacgtgtgccgcaccgacagcgcccgactgacgccactgcgctacgcccaccgcatcgaaaccggcctcaagatcagcgtcggcggcgatttcgcgtatggcgggcaactgaccaaaaacccgattcaccccgattgggagacgatctacggcccggccaccccgtacacattgcggcagctggccaccatccacacaccccggcagatgccgcgtcggcccgatcgggccgtgggcctgggccgcaacgtcaccatgttcgacgccacccggcgatgggcatacccgcagtggtggcaacaccgaaacggaaccggccgcgactgggaccatctcgtcctgcagcactgccacgccgtcaacaccgagttcacgacaccactgccgttcaccgaagtacgcgccaccgcgcaatccatctccaaatggatctggcgcaatttcaccgaagaacagtaccgagcccgacaagcgcatctcggtcaaaaaggcggcaaggcaacgacactcgccaaacaagaagccgtccgaaacaatgcaagaaagtacgacgaacatacgatgcgagaggcgattatctgatgggcggagccaaaaatccggtgcgccgaaagatgacggcagcagcagcagccgaaaaattcggtgcctccactcgcacaatccaacgcttgtttgctgagccgcgtgacgattacctcggccgtgcgaaagctcgccgtgacaaagctgtcgagctgcggaagcaggggttgaagtaccgggaaatcgccgaagcgatggaactctcgaccgggatcgtcggccgattactgcacgacgcccgcaggcacggcgagatttcagcggaggatctgtcggcgtaaccaagtcagcgggttgtcgggttccggccggcgctcggcactcggaccggccggcggatggtgttctgcctctggcgcagcgtcagctaccgccgaaggcctgtcatcgaccggcttcgactgaagtatgagcaacgtcacagcctgtgattggatgatccgctcacgctcgaccgctacctgttcagctgccgcccgctgggcatgagcaacggccaactctcgttcaa

SEQ ID NO: 113 - DNA, 8184 bp, Artificial Sequence
  Description of Artificial Sequence Synthetic vector pTip LCH2
gagctcgaccgcgcgggtcccggacggggaagagcggggagctttgccagagagcgacgacttccccttgcgttggtgattgccggtcagggcagccatccgccatcgtcgcgtagggtgtcacaccccaggaatcgcgtcactgaacacagcagccggtaggacgaccatgactgagttggacaccatcgcaaatccgtccgatcccgcggtgcagcggatcatcgatgtcaccaagccgtcacgatccaacataaagacaacgttgatcgaggacgtcgagcccctcatgcacagcatcgcggccggggtggagttcatcgaggtctacggcagcgacagcagtccttttccatctgagttgctggatctgtgcgggcggcagaacataccggtccgcctcatcgactcctcgatcgtcaaccagttgttcaagggggagcggaaggccaagacattcggcatcgcccgcgtccctcgcccggccaggttcggcgatatcgcgagccggcgtggggacgtcgtcgttctcgacggggtgaagatcgtcgggaacatcggcgcgatagtacgcacgtcgctcgcgctcggagcgtcggggatcatcctggtggacagtgacatcaccagcatcgcggaccggcgtctccaaagggccagccgaggttacgtcttctcccttcccgtcgttctctccggtcgcgaggaggccatcgccttcattcgggacagcggtatgcagctgatgacgctcaaggcggatggcgacatttccgtgaaggaactcggggacaatccggatcggctggccttgctgttcggcagcgaaaagggtgggccttccgacctgttcgaggaggcgtcttccgcctcggtttccatccccatgatgagccagaccgagtctctcaacgtttccgtttccctcggaatcgcgctgcacgagaggatcgacaggaatctcgcggccaaccgataagcgcctctgttcctcggacgctcggttcctcgacctcgattcgtcagtgatgatcacctcacacggcagcgatcaccactgacatatcgaggtcaacggtcgtggtccgggcgggcactcctcgaaggcgcggccgacgcccttgaacgactcgatgactctagagtaacgggctactccgtttaacggaccccgttctcacgctttaggcttgaccccggagcctgcatggggcattccgccgtgaacccggtggaatgcccccggcacccgggctttccagcaaagatcacctggcgccgatgagtaaggcgtacagaaccactccacaggaggaccgtcgagatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgtccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggactagaattgatctcctcgaccgccaattgggcatctgagaatcatctgcgtttctcgcacgcaacgtacttgcaacgttgcaactcctagtgttgtgaatcacaccccaccggggggtgggattgcagtcaccgatttggtgggtgcgcccaggaagatcacgtttacataggagcttgcaatgagctactccgtgggacaggtggccggcttcgccggagtgacggtgcgcacgctgcaccactacgacgacatcggcctgctcgtaccgagcgagcgcagccacgcgggccaccggcgctacagcgacgccgacctcgaccggctgcagcagatcctgttctaccgggagctgggcttcccgctcgacgaggtcgccgccctgctcgacgacccggccgcggacccgcgcgcgcacctgcgccgccagcacgagctgctgtccgcccggatcgggaaactgcagaagatggcggcggccgtggagcaggcgatggaggcacgcagcatgggaatcaacctcaccccggaggagaagttcgaggtcttcggcgacttcgaccccgaccagtacgaggaggaggtccgggaacgctgggggaacaccgacgcctaccgccagtccaaggagaagaccgcctcgtacaccaaggaggactggcagcgcatccaggacgaggccgacgagctcacccggcgcttcgtcgccctgatggacgcgggtgagcccgccgactccgagggggcgatggacgccgccgaggaccaccggcagggcatcgcccgcaaccactacgactgcgggtacgagatgcacacctgcctgggcgagatgtacgtgtccgacgaacgtttcacgcgaaacatcgacgccgccaagccgggcctcgccgcctacatgcgcgacgcgatcctcgccaacgccgtccggcacaccccctgagcggtggtcgtggcccgggtctcccgcccggtctcaccccacggctcactcccgggccacgaccaccgccgtcccgtacgcgcacacctcggtgcccacgtccgccgcctccgtcacgtcgaaacggaagatccccgggtaccgagctcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgactagagtcccgctgaggcggcgtagcaggtcagccgccccagcggtggtcaccaaccggggtggaacggcgccggtatcgggtgtgtccgtggcgctcattccaacctccgtgtgtttgtgcaggtttcgcgtgttgcagtccctcgcaccggcacccgcagcgaggggctcacgggtgccggtgggtcgactagttcagtgatggtgatggtgatgtcctcgagatctaagcttggatccgcggccgctacgtagaattcccatatgtatatctccttcttaaagttaaacaaaattatttctagacgccgtccacgctgcctcctcacgtgacgtgaggtgcaagcccggacgttccgcgtgccacgccgtgagccgccgcgtgccgtcggctccctcagcccgggcggccgtgggagcccgcctcgatatgtacacccgagaagctcccagcgtcctcctgggccgcgatactcgaccaccacgcacgcacaccgcactaacgattcggccggcgctcgattcggccggcgctcgattcggccggcgctcgattcggccggcgctcgattcggccggcgctcgattcggccgagcagaagagtgaacaaccaccgaccacgcttccgctctgcgcgccgtacccgacctacctcccgcagctcgaagcagctcccgggagtaccgccgtactcacccgcctgtgctcaccatccaccgacgcaaagcccaacccgagcacacctcttgcaccaaggtgccgaccgtggctttccgctcgcagggttccagaagaaatcgaacgatccagcgcggcaaggttcaaaaagcaggggttggtggggaggaggttttggggggtgtcgccgggatacctgatatggctttgttttgcgtagtcgaataattttccatatagcctcggcgcgtcggactcgaatagttgatgtgggcgggcacagttgccccatgaaatccgcaacggggggcgtgctgagcgatcggcaatgggcggatgcggtgttgcttccgcaccggccgttcgcgacgaacaacctccaacgaggtcagtaccggatgagccgcgacgacgcattggcaatgcggtacgtcgagcattcaccgcacgcgttgctcggatctatcgtcatcgactgcgatcacgttgacgccgcgatgcgcgcattcgagcaaccatccgaccatccggcgccgaactgggtcgcacaatcgccgtccggccgcgcacacatcggatggtggctcggccccaaccacgtgtgccgcaccgacagcgcccgactgacgccactgcgctacgcccaccgcatcgaaaccggcctcaagatcagcgtcggcggcgatttcgcgtatggcgggcaactgaccaaaaacccgattcaccccgattgggagacgatctacggcccggccaccccgtacacattgcggcagctggccaccatccacacaccccggcagatgccgcgtcggcccgatcgggccgtgggcctgggccgcaacgtcaccatgttcgacgccacccggcgatgggcatacccgcagtggtggcaacaccgaaacggaaccggccgcgactgggaccatctcgtcctgcagcactgccacgccgtcaacaccgagttcacgacaccactgccgttcaccgaagtacgcgccaccgcgcaatccatctccaaatggatctggcgcaatttcaccgaagaacagtaccgagcccgacaagcgcatctcggtcaaaaaggcggcaaggcaacgacactcgccaaacaagaagccgtccgaaacaatgcaagaaagtacgacgaacatacgatgcgagaggcgattatctgatgggcggagccaaaaatccggtgcgccgaaagatgacggcagcagcagcagccgaaaaattcggtgcctccactcgcacaatccaacgcttgtttgctgagccgcgtgacgattacctcggccgtgcgaaagctcgccgtgacaaagctgtcgagctgcggaagcaggggttgaagtaccgggaaatcgccgaagcgatggaactctcgaccgggatcgtcggccgattactgcacgacgcccgcaggcacggcgagatttcagcggaggatctgtcggcgtaaccaagtcagcgggttgtcgggttccggccggcgctcggcactcggaccggccggcggatggtgttctgcctctggcgcagcgtcagctaccgccgaaggcctgtcatcgaccggcttcgactgaagtatgagcaacgtcacagcctgtgattggatgatccgctcacgctcgaccgctacctgttcagctgccgcccgctgggcatgagcaacggccaactctcgttcaa

SEQ ID NO: 114 - protein, 6 aa, Artificial Sequence
  Description of Artificial Sequence Synthetic 6xHis tag
HHHHHH

SEQ ID NO: 115 - DNA, 422 bp, Artificial Sequence
  CDS (151)..(222)
  Description of Artificial Sequence Synthetic nucleotide sequence
gtgtacatatcgaggcgggctcccacggccgcccgggctgagggagccgacggcacgcggcggctcacggcgtggcacgcggaacgtccgggcttgcacctcacgtcacgtgaggaggcagcgtggacggcgtcagagaagggagcggccatgggccaccatcaccatcaccatatgggaattctacgtagcggccgcggatccaagcttagatctcgaggatgaactagtcgacccaccggcacccgtgagcccctcgctgcgggtgccggtgcgagggactgcaacacgcgaaacctgcacaaacacacggaggttggaatgagcgccacggacacacccgataccggcgccgttccaccccggttggtgaccaccgctggggcggctgacctgctacgccgcctcagcgggactctagt

SEQ ID NO: 116 - protein, 24 aa, Artificial Sequence
  Description of Artificial Sequence Synthetic coded protein sequence
MGHHHHHHMGILRSGRGSKLRSRG

SEQ ID NO: 117 - DNA, 416 bp, Artificial Sequence
  CDS (151)..(216)
  Description of Artificial Sequence Synthetic nucleotide sequence
gtgtacatatcgaggcgggctcccacggccgcccgggctgagggagccgacggcacgcggcggctcacggcgtggcacgcggaacgtccgggcttgcacctcacgtcacgtgaggaggcagcgtggacggcgtcagagaagggagcggccatgggaattctacgtagcggccgcggatccaagcttagatctcgaggacatcaccatcaccatcactgaactagtcgacccaccggcacccgtgagcccctcgctgcgggtgccggtgcgagggactgcaacacgcgaaacctgcacaaacacacggaggttggaatgagcgccacggacacacccgataccggcgccgttccaccccggttggtgaccaccgctggggcggctgacctgctacgccgcctcagcgggactctagt

SEQ ID NO: 118 - protein, 22 aa, Artificial Sequence
  Description of Artificial Sequence Synthetic coded protein sequence
MGILRSGRGSKLRSRGHHHHHH

SEQ ID NO: 119 - DNA, 425 bp, Artificial Sequence
  CDS (151)..(225)
  Description of Artificial Sequence Synthetic nucleotide sequence
gtgtacatatcgaggcgggctcccacggccgcccgggctgagggagccgacggcacgcggcggctcacggcgtggcacgcggaacgtccgggcttgcacctcacgtcacgtgaggaggcagcgtggacggcgtcagagaagggagcgcatatgggccatcaccatcaccatcacgccatgggaattctacgtagcggccgcggatccaagcctagatctcgaggatgaactagtcgacccaccggcacccgtgagcccctcgctgcgggtgccggtgcgagggactgcaacacgcgaaacctgcacaaacacacggaggttggaatgagcgccacggacacacccgataccggcgccgttccaccccggttggtgaccaccgctggggcggctgacctgctacgccgcctcagcgggactctagt

SEQ ID NO: 120 - protein, 25 aa, Artificial Sequence
  Description of Artificial Sequence Synthetic coded protein sequence
MGHHHHHHAMGILRSGRGSKPRSRG

SEQ ID NO: 121 - DNA, 416 bp, Artificial Sequence
  CDS (151)..(216)
  Description of Artificial Sequence Synthetic nucleotide sequence
gtgtacatatcgaggcgggctcccacggccgcccgggctgagggagccgacggcacgcggcggctcacggcgtggcacgcggaacgtccgggcttgcacctcacgtcacgtgaggaggcagcgtggacggcgtcagagaagggagcgcatatgggaattctacgtagcggccgcggatccaagcttagatctcgaggacatcaccatcaccatcactgaactagtcgacccaccggcacccgtgagcccctcgctgcgggtgccggtgcgagggactgcaacacgcgaaacctgcacaaacacacggaggttggaatgagcgccacggacacacccgataccggcgccgttccaccccggttggtgaccaccgctggggcggctgacctgctacgccgcctcagcgggactctagt

SEQ ID NO: 122 - protein, 22 aa, Artificial Sequence
  Description of Artificial Sequence Synthetic coded protein sequence
MGILRSGRGSKLRSRGHHHHHH

SEQ ID NO: 123 - DNA, 81 bp, Artificial Sequence
  CDS (3)..(68)
  Description of Artificial Sequence Synthetic nucleotide sequence
ccatgggaattctacgtagcggccgcggatccaagcttagatctctcgagcatcaccatcaccatcactgaactagtcgac

SEQ ID NO: 124 - protein, 22 aa, Artificial Sequence
  Description of Artificial Sequence Synthetic coded protein sequence
MGILRSGRGSKLRSLEHHHHHH

SEQ ID NO: 125 - DNA, 82 bp, Artificial Sequence
  CDS (4)..(69)
  Description of Artificial Sequence Synthetic nucleotide sequence
catatgggaattctacgtagcggccgcggatccaagcttagatctctcgagcatcaccatcaccatcactgaactagtcgac

SEQ ID NO: 126 - protein, 22 aa, Artificial Sequence
  Description of Artificial Sequence Synthetic coded protein sequence
MGILRSGRGSKLRSLEHHHHHH

SEQ ID NO: 127 - DNA, 124 bp, Artificial Sequence
  Description of Artificial Sequence Synthetic nucleotide sequence
cgcccgggctgagggagccgacggcacgcggcggctcacggcgtggcacgcggaacgtccgggcttgcacctcacgtcacgtgaggaggcagcgtggacggcgtcagagaagggagcggccatg

SEQ ID NO: 128 - DNA, 22 bp, Artificial Sequence
  Description of Artificial Sequence Synthetic oligonucleotide
gtcagagaagggagcggccatg

SEQ ID NO: 129 - DNA, 45 bp, Artificial Sequence
  Description of Artificial Sequence Synthetic oligonucleotide
gtctagaaataattttgtttaactttaagaaggagatataccatg

SEQ ID NO: 130 - DNA, 42 bp, Artificial Sequence
  Description of Artificial Sequence Synthetic oligonucleotide
gtctagaaataattttgtttaactttaagaaggagatatacc

SEQ ID NO: 131 - DNA, 43 bp, Artificial Sequence
  Description of Artificial Sequence Synthetic oligonucleotide
gtctagaaataattttgtttaactttaagaaggagatatacat